SEQUENCE LISTING (388 sequences)

SEQ ID NO: 1 - protein, 15 aa, Homo sapiens
  Residues 2- 16 RhD
SSKYPRSVRRCLPLW

SEQ ID NO: 2 - protein, 15 aa, Homo sapiens
  Residues 12-26 RhD
CLPLWALTLEAALIL

SEQ ID NO: 3 - protein, 15 aa, Homo sapiens
  Residue 22- 36 RhD
AALILLFYFFTHYDA

SEQ ID NO: 4 - protein, 15 aa, Homo sapiens
  Residues 32-46 RhD
THYDASLEDQKGLVA

SEQ ID NO: 5 - protein, 15 aa, Homo sapiens
  Residue 42-56 RhD
KGLVASYQVGQDLTV

SEQ ID NO: 6 - protein, 15 aa, Homo sapiens
  Residue 52-66 RhD
QDLTVMAAIGLGFLT

SEQ ID NO: 7 - protein, 15 aa, Homo sapiens
  Residue 57-71 RhD
MAAIGLGFLTSSFRR

SEQ ID NO: 8 - protein, 15 aa, Homo sapiens
  Residue 62-76 RhD
LGFLTSSFRRHSWSS

SEQ ID NO: 9 - protein, 15 aa, Homo sapiens
  Residue 67-81 RhD
SSFRRHSWSSVAFNL

SEQ ID NO: 10 - protein, 15 aa, Homo sapiens
  Residue 72-86 RhD
HSWSSVAFNLFMLAL

SEQ ID NO: 11 - protein, 15 aa, Homo sapiens
  Residue 82-96 RhD
FMLALGVQWAILLDG

SEQ ID NO: 12 - protein, 15 aa, Homo sapiens
  Residue 92-106 RhD
ILLDGFLSQFPSGKV

SEQ ID NO: 13 - protein, 15 aa, Homo sapiens
  Residue 97-111 RhD
FLSQFPSGKVVITLF

SEQ ID NO: 14 - protein, 15 aa, Homo sapiens
  Residue 102-116 RhD
PSGKVVITLFSIRLA

SEQ ID NO: 15 - protein, 15 aa, Homo sapiens
  Residue 107-121 RhD
VITLFSIRLATMSAL

SEQ ID NO: 16 - protein, 15 aa, Homo sapiens
  Residue 112-126 RhD
SIRLATMSALSVLIS

SEQ ID NO: 17 - protein, 15 aa, Homo sapiens
  Residue 117-131 RhD
TMSALSVLISVDAVL

SEQ ID NO: 18 - protein, 15 aa, Homo sapiens
  Residue 122-136 RhD
SVLISVDAVLGKVNL

SEQ ID NO: 19 - protein, 15 aa, Homo sapiens
  Residue 127-141 RhD
VDAVLGKVNLAQLVV

SEQ ID NO: 20 - protein, 15 aa, Homo sapiens
  Residue 132-146 RhD
GKVNLAQLVVMVLVE

SEQ ID NO: 21 - protein, 15 aa, Homo sapiens
  Residue 142-156 RhD
MVLVEVTALGNLRMV

SEQ ID NO: 22 - protein, 15 aa, Homo sapiens
  Residue 147 - 161 RhD
VTALGNLRMVISNIF

SEQ ID NO: 23 - protein, 15 aa, Homo sapiens
  Residue 152 - 166 RhD
NLRMVISNIFNTDYH

SEQ ID NO: 24 - protein, 15 aa, Homo sapiens
  Residue 157-171 RhD
ISNIFNTDYHMNMMH

SEQ ID NO: 25 - protein, 15 aa, Homo sapiens
  Residue 162-176 RhD
NTDYHMNMMHIYVFA

SEQ ID NO: 26 - protein, 15 aa, Homo sapiens
  Residue 167-181 RhD
MNMMHIYVFAAYFGL

SEQ ID NO: 27 - protein, 15 aa, Homo sapiens
  Residue 172-186 RhD
IYVFAAYFGLSVAWC

SEQ ID NO: 28 - protein, 15 aa, Homo sapiens
  Residue 177-191 RhD
AYFGLSVAWCLPKPL

SEQ ID NO: 29 - protein, 15 aa, Homo sapiens
  Residue 182-196 RhD
SVAWCLPKPLPEGTE

SEQ ID NO: 30 - protein, 15 aa, Homo sapiens
  Residue 187-201 RhD
LPKPLPEGTEDKDQT

SEQ ID NO: 31 - protein, 15 aa, Homo sapiens
  Residue 192-206 RhD
PEGTEDKDQTATIPS

SEQ ID NO: 32 - protein, 15 aa, Homo sapiens
  Residue 197-211 RhD
DKDQTATIPSLSAML

SEQ ID NO: 33 - protein, 15 aa, Homo sapiens
  Residue 202-216 RhD
ATIPSLSAMLGALFL

SEQ ID NO: 34 - protein, 15 aa, Homo sapiens
  Residue 207-221 RhD
LSAMLGALFLWMFWP

SEQ ID NO: 35 - protein, 15 aa, Homo sapiens
  Residue 212-226 RhD
GALFLWMFWPSFNSA

SEQ ID NO: 36 - protein, 15 aa, Homo sapiens
  Residue 217-231 RhD
WMFWPSFNSALLRSP

SEQ ID NO: 37 - protein, 15 aa, Homo sapiens
  Residue 222-236 RhD
SFNSALLRSPIERKN

SEQ ID NO: 38 - protein, 15 aa, Homo sapiens
  Residue 227-241 RhD
LLRSPIERKNAVFNT

SEQ ID NO: 39 - protein, 15 aa, Homo sapiens
  Residue 232-246 RhD
IERKNAVFNTYYALA

SEQ ID NO: 40 - protein, 15 aa, Homo sapiens
  Residue 237-251 RhD
AVFNTYYAVAVSVVT

SEQ ID NO: 41 - protein, 15 aa, Homo sapiens
  Residue 242-256 RhD
YYAVAVSVVTAISGS

SEQ ID NO: 42 - protein, 15 aa, Homo sapiens
  Residue 252-266 RhD
AISGSSLAHPQGKIS

SEQ ID NO: 43 - protein, 15 aa, Homo sapiens
  Residue 257-271 RhD
SLAHPQGKISKTYVH

SEQ ID NO: 44 - protein, 15 aa, Homo sapiens
  Residue 262-276 RhD
QGKISKTYVHSAVLA

SEQ ID NO: 45 - protein, 15 aa, Homo sapiens
  Residue 267-281 RhD
KTYVHSAVLAGGVAV

SEQ ID NO: 46 - protein, 15 aa, Homo sapiens
  Residue 272-286 RhD
SAVLAGGVAVGTSCH

SEQ ID NO: 47 - protein, 15 aa, Homo sapiens
  Residue 282-296 RhD
GTSCHLIPSPWLAMV

SEQ ID NO: 48 - protein, 15 aa, Homo sapiens
  Residue 292-306 RhD
WLAMVLGLVAGLISV

SEQ ID NO: 49 - protein, 15 aa, Homo sapiens
  Residue 297-311 RhD
LGLVAGLISVGGAKY

SEQ ID NO: 50 - protein, 15 aa, Homo sapiens
  Residue 302-316 RhD
GLISVGGAKYLPGCC

SEQ ID NO: 51 - protein, 15 aa, Homo sapiens
  Residue 307-321 RhD
GGAKYLPGCCNRVLG

SEQ ID NO: 52 - protein, 15 aa, Homo sapiens
  Residue 312-326 RhD
LPGCCNRVLGIPHSS

SEQ ID NO: 53 - protein, 15 aa, Homo sapiens
  Residue 317-331 RhD
NRVLGIPHSSIMGYN

SEQ ID NO: 54 - protein, 15 aa, Homo sapiens
  Residue 322-336 RhD
IPHSSIMGYNFSLLG

SEQ ID NO: 55 - protein, 15 aa, Homo sapiens
  Residue 327-341 RhD
IMGYNFSLLGLLGEI

SEQ ID NO: 56 - protein, 15 aa, Homo sapiens
  Residue 332-346 RhD
FSLLGLLGEIIYIVL

SEQ ID NO: 57 - protein, 15 aa, Homo sapiens
  Residue 337-351 RhD
LLGEIIYIVLLVLDT

SEQ ID NO: 58 - protein, 15 aa, Homo sapiens
  Residue 342-356 RhD
IYIVLLVLDTVGAGN

SEQ ID NO: 59 - protein, 15 aa, Homo sapiens
  Residue 347-361 RhD
LVLDTVGAGNGMIGF

SEQ ID NO: 60 - protein, 15 aa, Homo sapiens
  Residue 352-366 RhD
VGAGNGMIGFQVLLS

SEQ ID NO: 61 - protein, 15 aa, Homo sapiens
  Residue 362-376 RhD
QVLLSIGELSLAIVI

SEQ ID NO: 62 - protein, 15 aa, Homo sapiens
  Residue 372-386 RhD
LAIVIALTSGLLTGL

SEQ ID NO: 63 - protein, 15 aa, Homo sapiens
  Residue 382-396 RhD
LLTGLLLNLKIWKAP

SEQ ID NO: 64 - protein, 15 aa, Homo sapiens
  Residue 387-401 RhD
LLNLKIWKAPHEAKY

SEQ ID NO: 65 - protein, 15 aa, Homo sapiens
  Residue 392-406 RhD
IWKAPHEAKYFDDQV

SEQ ID NO: 66 - protein, 15 aa, Homo sapiens
  Residue 397-411 RhD
HEAKYFDDQVFWKFP

SEQ ID NO: 67 - protein, 15 aa, Homo sapiens
  Residue 402-416 RhD
FDDQVFWKFPHLAVG

SEQ ID NO: 68 - protein, 15 aa, Homo sapiens
  Residue 403-417 RhD
DDQVFWKFPHLAVGF

SEQ ID NO: 69 - protein, 15 aa, Homo sapiens
  Residue 207-221 RhD
LSAMLGALFLWIFWP

SEQ ID NO: 70 - protein, 14 aa, Homo sapiens
  Residue 212-226 RhD
GALFLWIFWPSFNS

SEQ ID NO: 71 - protein, 15 aa, Homo sapiens
  Residue 217-231 RhD
WIFWPSFNSALLRSP

SEQ ID NO: 72 - protein, 15 aa, Homo sapiens
  Residues 2-16 RhcE
SSKYPRSVRRCLPLW

SEQ ID NO: 73 - protein, 15 aa, Homo sapiens
  Residues 12-26 RhcE
CLPLWALTLEAALIL

SEQ ID NO: 74 - protein, 15 aa, Homo sapiens
  Residue 22-36 RhcE
AALILLFYFFTHYDA

SEQ ID NO: 75 - protein, 15 aa, Homo sapiens
  Residues 32-46 RhcE
THYDASLEDQKGLVA

SEQ ID NO: 76 - protein, 15 aa, Homo sapiens
  Residue 42-56 RhcE
KGLVASYQVGQDLTV

SEQ ID NO: 77 - protein, 15 aa, Homo sapiens
  Residue 52-66 RhcE
QDLTVMAALGLGFLT

SEQ ID NO: 78 - protein, 15 aa, Homo sapiens
  Residue 57-71 RhcE
MAALGLGFLTSNFRR

SEQ ID NO: 79 - protein, 15 aa, Homo sapiens
  Residue 62-76 RhcE
LGFLTSNFRRHSWSS

SEQ ID NO: 80 - protein, 15 aa, Homo sapiens
  Residue 67-81 RhcE
SNFRRHSWSSVAFNL

SEQ ID NO: 81 - protein, 15 aa, Homo sapiens
  Residue 72-86 RhcE
HSWSSVAFNLFMLAL

SEQ ID NO: 82 - protein, 15 aa, Homo sapiens
  Residue 82-96 RhcE
FMLALGVQWAILLDG

SEQ ID NO: 83 - protein, 15 aa, Homo sapiens
  Residue 92-106 RhcE
ILLDGFLSQFPPGKV

SEQ ID NO: 84 - protein, 15 aa, Homo sapiens
  Residue 97-111 RhcE
FLSQFPPGKVVITLF

SEQ ID NO: 85 - protein, 15 aa, Homo sapiens
  Residue 102-116 RhcE
PPGKVVITLFSIRLA

SEQ ID NO: 86 - protein, 15 aa, Homo sapiens
  Residue 107-121 RhcE
VITLFSIRLATMSAM

SEQ ID NO: 87 - protein, 15 aa, Homo sapiens
  Residue 112-126 RhcE
SIRLATMSAMSVLIS

SEQ ID NO: 88 - protein, 15 aa, Homo sapiens
  Residue 117-131 RhcE
TMSAMSVLISAGAVL

SEQ ID NO: 89 - protein, 15 aa, Homo sapiens
  Residue 122-136 RhcE
SVLISAGAVLGKVNL

SEQ ID NO: 90 - protein, 15 aa, Homo sapiens
  Residue 127-141 RhcE
AGAVLGKVNLAQLVV

SEQ ID NO: 91 - protein, 15 aa, Homo sapiens
  Residue 132-146 RhcE
GKVNLAQLVVMVLVE

SEQ ID NO: 92 - protein, 15 aa, Homo sapiens
  Residue 142-156 RhcE
MVLVEVTALGTLRMV

SEQ ID NO: 93 - protein, 15 aa, Homo sapiens
  Residue 147-161 RhcE
VTALGTLRMVISNIF

SEQ ID NO: 94 - protein, 15 aa, Homo sapiens
  Residue 152-166 RhcE
TLRMVISNIFNTDYH

SEQ ID NO: 95 - protein, 15 aa, Homo sapiens
  Residue 157-171 RhcE
ISNIFNTDYHMNLRH

SEQ ID NO: 96 - protein, 15 aa, Homo sapiens
  Residue 162-176 RhcE
NTDYHMNLRHIYVFA

SEQ ID NO: 97 - protein, 15 aa, Homo sapiens
  Residue 167-181 RhcE
MNLRHIYVFAAYFGL

SEQ ID NO: 98 - protein, 15 aa, Homo sapiens
  Residue 172-186 RhcE
IYVFAAYFGLTVAWC

SEQ ID NO: 99 - protein, 15 aa, Homo sapiens
  Residue 177-191 RhcE
AYFGLTVAWCLPKPL

SEQ ID NO: 100 - protein, 15 aa, Homo sapiens
  Residue 182-196 RhcE
TVAWCLPKPLPKGTE

SEQ ID NO: 101 - protein, 15 aa, Homo sapiens
  Residue 187-201 RhcE
LPKPLKEGTEDKDQR

SEQ ID NO: 102 - protein, 15 aa, Homo sapiens
  Residue 192-206 RhcE
PKGTEDNDQRATIPS

SEQ ID NO: 103 - protein, 15 aa, Homo sapiens
  Residue 197-211 RhcE
DNDQRATIPSLSAML

SEQ ID NO: 104 - protein, 15 aa, Homo sapiens
  Residue 202-216 RhcE
ATIPSLSAMLGALFL

SEQ ID NO: 105 - protein, 15 aa, Homo sapiens
  Residue 207-221 RhcE
LSAMLGALFLWMFWP

SEQ ID NO: 106 - protein, 15 aa, Homo sapiens
  Residue 212-226 RhcE
GALFLWMFWPSVNSP

SEQ ID NO: 107 - protein, 15 aa, Homo sapiens
  Residue 217-231 RhcE
WMFWPSVNSPLLRSP

SEQ ID NO: 108 - protein, 15 aa, Homo sapiens
  Residue 222-236 RhcE
SVNSPLLRSPIQRKN

SEQ ID NO: 109 - protein, 15 aa, Homo sapiens
  Residue 227-241 RhcE
LLRSPIQRKNAMFNT

SEQ ID NO: 110 - protein, 15 aa, Homo sapiens
  Residue 232-246 RhcE
IQRKNAMFNTYYALA

SEQ ID NO: 111 - protein, 15 aa, Homo sapiens
  Residue 237-251 RhcE
AMFNTYYALAVSVVT

SEQ ID NO: 112 - protein, 15 aa, Homo sapiens
  Residue 242-256 RhcE
YYAVAVSVVTAISGS

SEQ ID NO: 113 - protein, 15 aa, Homo sapiens
  Residue 252-266 RhcE
AISGSSLAHPQRKIS

SEQ ID NO: 114 - protein, 15 aa, Homo sapiens
  Residue 257-271 RhcE
SLAHPQRKISMTYVH

SEQ ID NO: 115 - protein, 15 aa, Homo sapiens
  Residue 262-276 RhcE
QRKISMTYVHSAVLA

SEQ ID NO: 116 - protein, 15 aa, Homo sapiens
  Residue 267-281 RhcE
MTYVHSAVLAGGVAV

SEQ ID NO: 117 - protein, 15 aa, Homo sapiens
  Residue 272-286 RhcE
SAVLAGGVAVGTSCH

SEQ ID NO: 118 - protein, 15 aa, Homo sapiens
  Residue 282-296 RhcE
GTSCHLIPSPWLAMV

SEQ ID NO: 119 - protein, 15 aa, Homo sapiens
  Residue 292-306 RhcE
WLAMVLGLVAGLISI

SEQ ID NO: 120 - protein, 15 aa, Homo sapiens
  Residue 297-311 RhcE
LGLVAGLISIGGAKY

SEQ ID NO: 121 - protein, 15 aa, Homo sapiens
  Residue 302-316 RhcE
GLISIGGAKCLPVCC

SEQ ID NO: 122 - protein, 15 aa, Homo sapiens
  Residue 307-321 RhcE
GGAKCLPVCCNRVLG

SEQ ID NO: 123 - protein, 15 aa, Homo sapiens
  Residue 312-326 RhcE
LPVCCNRVLGIHHIS

SEQ ID NO: 124 - protein, 15 aa, Homo sapiens
  Residue 317-331 RhcE
NRVLGIHHISVMHSI

SEQ ID NO: 125 - protein, 15 aa, Homo sapiens
  Residue 322-336 RhcE
IHHISVMHSIFSLLG

SEQ ID NO: 126 - protein, 15 aa, Homo sapiens
  Residue 327-341 RhcE
IMHSIFSLLGLLGEI

SEQ ID NO: 127 - protein, 15 aa, Homo sapiens
  Residue 332-346 RhcE
FSLLGLLGEITYIVL

SEQ ID NO: 128 - protein, 15 aa, Homo sapiens
  Residue 337-351 RhcE
LLGEITYIVLLVLHT

SEQ ID NO: 129 - protein, 15 aa, Homo sapiens
  Residue 342-356 RhcE
TYIVLLVLHTVWNGN

SEQ ID NO: 130 - protein, 15 aa, Homo sapiens
  Residue 347-361 RhcE
LVLHTVWNGNGMIGF

SEQ ID NO: 131 - protein, 15 aa, Homo sapiens
  Residue 352-366 RhcE
VGNGNGMIGFQVLLS

SEQ ID NO: 132 - protein, 15 aa, Homo sapiens
  Residue 362-376 RhcE
QVLLSIGELSLAIVI

SEQ ID NO: 133 - protein, 15 aa, Homo sapiens
  Residue 372-386 RhcE
LAIVIALTSGLLTGL

SEQ ID NO: 134 - protein, 15 aa, Homo sapiens
  Residue 382-396 RhcE
LLTGLLLNLKIWKAP

SEQ ID NO: 135 - protein, 15 aa, Homo sapiens
  Residue 387-401 RhcE
LLNLKIWKAPHVAKY

SEQ ID NO: 136 - protein, 15 aa, Homo sapiens
  Residue 392-406 RhcE
IWKAPHVAKYFDDQV

SEQ ID NO: 137 - protein, 15 aa, Homo sapiens
  Residue 397-411 RhcE
HVAKYFDDQVFWKFP

SEQ ID NO: 138 - protein, 15 aa, Homo sapiens
  Residue 402-416 RhcE
FDDQVFWKFPHLAVG

SEQ ID NO: 139 - protein, 15 aa, Homo sapiens
  Residue 403-417 RhcE
DDQVFWKFPHLAVGF

SEQ ID NO: 140 - protein, 15 aa, Homo sapiens
  Residues 2- 16 Rhce
SSKYPRSVRRCLPLW

SEQ ID NO: 141 - protein, 15 aa, Homo sapiens
  Residues 12-26 Rhce
CLPLWALTLEAALIL

SEQ ID NO: 142 - protein, 15 aa, Homo sapiens
  Residue 22-36 Rhce
AALILLFYFFTHYDA

SEQ ID NO: 143 - protein, 15 aa, Homo sapiens
  Residues 32-46 Rhce
THYDASLEDQKGLVA

SEQ ID NO: 144 - protein, 15 aa, Homo sapiens
  Residue 42-56 Rhce
KGLVASYQVGQDLTV

SEQ ID NO: 145 - protein, 15 aa, Homo sapiens
  Residue 52-66 Rhce
QDLTVMAALGLGFLT

SEQ ID NO: 146 - protein, 15 aa, Homo sapiens
  Residue 57-71 Rhce
MAALGLGFLTSNFRR

SEQ ID NO: 147 - protein, 15 aa, Homo sapiens
  Residue 62-76 Rhce
LGFLTSNFRRHSWSS

SEQ ID NO: 148 - protein, 15 aa, Homo sapiens
  Residue 67-81 Rhce
SNFRRHSWSSVAFNL

SEQ ID NO: 149 - protein, 15 aa, Homo sapiens
  Residue 72-86 Rhce
HSWSSVAFNLFMLAL

SEQ ID NO: 150 - protein, 15 aa, Homo sapiens
  Residue 82-96 Rhce
FMLALGVQWAILLDG

SEQ ID NO: 151 - protein, 15 aa, Homo sapiens
  Residue 92-106 Rhce
ILLDGFLSQFPPGKV

SEQ ID NO: 152 - protein, 15 aa, Homo sapiens
  Residue 97-111 Rhce
FLSQFPPGKVVITLF

SEQ ID NO: 153 - protein, 15 aa, Homo sapiens
  Residue 102-116 Rhce
PPGKVVITLFSIRLA

SEQ ID NO: 154 - protein, 15 aa, Homo sapiens
  Residue 107-121 Rhce
VITLFSIRLATMSAM

SEQ ID NO: 155 - protein, 15 aa, Homo sapiens
  Residue 112-126 Rhce
SIRLATMSAMSVLIS

SEQ ID NO: 156 - protein, 15 aa, Homo sapiens
  Residue 117-131 Rhce
TMSAMSVLISAGAVL

SEQ ID NO: 157 - protein, 15 aa, Homo sapiens
  Residue 122-136 Rhce
SVLISAGAVLGKVNL

SEQ ID NO: 158 - protein, 15 aa, Homo sapiens
  Residue 127-141 Rhce
AGAVLGKVNLAQLVV

SEQ ID NO: 159 - protein, 15 aa, Homo sapiens
  Residue 132-146 Rhce
GKVNLAQLVVMVLVE

SEQ ID NO: 160 - protein, 15 aa, Homo sapiens
  Residue 142-156 Rhce
MVLVEVTALGTLRMV

SEQ ID NO: 161 - protein, 15 aa, Homo sapiens
  Residue 147-161 Rhce
VTALGTLRMVISNIF

SEQ ID NO: 162 - protein, 15 aa, Homo sapiens
  Residue 152-166 Rhce
TLRMVISNIFNTDYH

SEQ ID NO: 163 - protein, 15 aa, Homo sapiens
  Residue 157-171 Rhce
ISNIFNTDYHMNLRH

SEQ ID NO: 164 - protein, 15 aa, Homo sapiens
  Residue 162-176 Rhce
NTDYHMNLRHIYVFA

SEQ ID NO: 165 - protein, 15 aa, Homo sapiens
  Residue 167-181 Rhce
MNLRHIYVFAAYFGL

SEQ ID NO: 166 - protein, 15 aa, Homo sapiens
  Residue 172-186 Rhce
IYVFAAYFGLTVAWC

SEQ ID NO: 167 - protein, 15 aa, Homo sapiens
  Residue 177-191 Rhce
AYFGLTVAWCLPKPL

SEQ ID NO: 168 - protein, 15 aa, Homo sapiens
  Residue 182-196 Rhce
TVAWCLPKPLPKGTE

SEQ ID NO: 169 - protein, 15 aa, Homo sapiens
  Residue 187-201 Rhce
LPKPLKEGTEDKDQR

SEQ ID NO: 170 - protein, 15 aa, Homo sapiens
  Residue 192-206 Rhce
PKGTEDNDQRATIPS

SEQ ID NO: 171 - protein, 15 aa, Homo sapiens
  Residue 197-211 Rhce
DNDQRATIPSLSAML

SEQ ID NO: 172 - protein, 15 aa, Homo sapiens
  Residue 202-216 Rhce
ATIPSLSAMLGALFL

SEQ ID NO: 173 - protein, 15 aa, Homo sapiens
  Residue 207-221 Rhce
LSAMLGALFLWMFWP

SEQ ID NO: 174 - protein, 15 aa, Homo sapiens
  Residue 212-226 Rhce
GALFLWMFWPSVNSA

SEQ ID NO: 175 - protein, 15 aa, Homo sapiens
  Residue 217-231 Rhce
WMFWPSVNSALLRSP

SEQ ID NO: 176 - protein, 15 aa, Homo sapiens
  Residue 222-236 Rhce
SVNAPLLRSPIERKN

SEQ ID NO: 177 - protein, 15 aa, Homo sapiens
  Residue 227-241 Rhce
LLRSPIQRKNAMFNT

SEQ ID NO: 178 - protein, 15 aa, Homo sapiens
  Residue 232-246 Rhce
IQRKNAMFNTYYALA

SEQ ID NO: 179 - protein, 15 aa, Homo sapiens
  Residue 237-251 Rhce
AMFNTYYALAVSVVT

SEQ ID NO: 180 - protein, 15 aa, Homo sapiens
  Residue 242-256 Rhce
YYAVAVSVVTAISGS

SEQ ID NO: 181 - protein, 15 aa, Homo sapiens
  Residue 252-266 Rhce
AISGSSLAHPQRKIS

SEQ ID NO: 182 - protein, 15 aa, Homo sapiens
  Residue 257-271 Rhce
SLAHPQRKISMTYVH

SEQ ID NO: 183 - protein, 15 aa, Homo sapiens
  Residue 262-276 Rhce
QRKISMTYVHSAVLA

SEQ ID NO: 184 - protein, 15 aa, Homo sapiens
  Residue 267-281 Rhce
MTYVHSAVLAGGVAV

SEQ ID NO: 185 - protein, 15 aa, Homo sapiens
  Residue 272-286 Rhce
SAVLAGGVAVGTSCH

SEQ ID NO: 186 - protein, 15 aa, Homo sapiens
  Residue 282-296 Rhce
GTSCHLIPSPWLAMV

SEQ ID NO: 187 - protein, 15 aa, Homo sapiens
  Residue 292-306 Rhce
WLAMVLGLVAGLISI

SEQ ID NO: 188 - protein, 15 aa, Homo sapiens
  Residue 297-311 Rhce
LGLVAGLISIGGAKY

SEQ ID NO: 189 - protein, 15 aa, Homo sapiens
  Residue 302-316 Rhce
GLISIGGAKCLPVCC

SEQ ID NO: 190 - protein, 15 aa, Homo sapiens
  Residue 307-321 Rhce
GGAKCLPVCCNRVLG

SEQ ID NO: 191 - protein, 15 aa, Homo sapiens
  Residue 312-326 Rhce
LPVCCNRVLGIHHIS

SEQ ID NO: 192 - protein, 15 aa, Homo sapiens
  Residue 317-331 Rhce
NRVLGIHHISVMHSI

SEQ ID NO: 193 - protein, 15 aa, Homo sapiens
  Residue 322-336 Rhce
IHHISVMHSIFSLLG

SEQ ID NO: 194 - protein, 15 aa, Homo sapiens
  Residue 327-341 Rhce
IMHSIFSLLGLLGEI

SEQ ID NO: 195 - protein, 15 aa, Homo sapiens
  Residue 332-346 Rhce
FSLLGLLGEITYIVL

SEQ ID NO: 196 - protein, 15 aa, Homo sapiens
  Residue 337-351 Rhce
LLGEITYIVLLVLHT

SEQ ID NO: 197 - protein, 15 aa, Homo sapiens
  Residue 342-356 Rhce
TYIVLLVLHTVWNGN

SEQ ID NO: 198 - protein, 15 aa, Homo sapiens
  Residue 347-361 Rhce
LVLHTVWNGNGMIGF

SEQ ID NO: 199 - protein, 15 aa, Homo sapiens
  Residue 352-366 Rhce
VWNGNGMIGFQVLLS

SEQ ID NO: 200 - protein, 15 aa, Homo sapiens
  Residue 362-376 Rhce
QVLLSIGELSLAIVI

SEQ ID NO: 201 - protein, 15 aa, Homo sapiens
  Residue 372-386 Rhce
LAIVIALTSGLLTGL

SEQ ID NO: 202 - protein, 15 aa, Homo sapiens
  Residue 382-396 Rhce
LLTGLLLNLKIWKAP

SEQ ID NO: 203 - protein, 15 aa, Homo sapiens
  Residue 387-401 Rhce
LLNLKIWKAPHVAKY

SEQ ID NO: 204 - protein, 15 aa, Homo sapiens
  Residue 392-406 Rhce
IWKAPHVAKYFDDQV

SEQ ID NO: 205 - protein, 15 aa, Homo sapiens
  Residue 397-411 Rhce
HVAKYFDDQVFWKFP

SEQ ID NO: 206 - protein, 15 aa, Homo sapiens
  Residue 402-416 Rhce
FDDQVFWKFPHLAVG

SEQ ID NO: 207 - protein, 15 aa, Homo sapiens
  Residue 403-417 Rhce
DDQVFWKFPHLAVGF

SEQ ID NO: 208 - protein, 15 aa, Homo sapiens
  Residues 2-16 RhCe
SSKYPRSVRRCLPLC

SEQ ID NO: 209 - protein, 15 aa, Homo sapiens
  Residues 12-26 RhCe
CLPLCALTLEAALIL

SEQ ID NO: 210 - protein, 15 aa, Homo sapiens
  Residue 22-36 RhCe
AALILLFYFFTHYDA

SEQ ID NO: 211 - protein, 15 aa, Homo sapiens
  Residues 32-46 RhCe
THYDASLEDQKGLVA

SEQ ID NO: 212 - protein, 15 aa, Homo sapiens
  Residue 42-56 RhCe
KGLVASYQVGQDLTV

SEQ ID NO: 213 - protein, 15 aa, Homo sapiens
  Residue 52-66 RhCe
QDLTVMAAIGLGFLT

SEQ ID NO: 214 - protein, 15 aa, Homo sapiens
  Residue 57-71 RhCe
MAAIGLGFLTSNSRR

SEQ ID NO: 215 - protein, 15 aa, Homo sapiens
  Residue 62-76 RhCe
LGFLTSSFRRHSWSS

SEQ ID NO: 216 - protein, 15 aa, Homo sapiens
  Residue 67-81 RhCe
SSFRRHSWSSVAFNL

SEQ ID NO: 217 - protein, 15 aa, Homo sapiens
  Residue 72-86 RhCe
HSWSSVAFNLFMLAL

SEQ ID NO: 218 - protein, 15 aa, Homo sapiens
  Residue 82-96 RhCe
FMLALGVQWAILLDG

SEQ ID NO: 219 - protein, 15 aa, Homo sapiens
  Residue 92-106 RhCe
ILLDGFLSQFPSGKV

SEQ ID NO: 220 - protein, 15 aa, Homo sapiens
  Residue 97-111 RhCe
FLSQFPSGKVVITLF

SEQ ID NO: 221 - protein, 15 aa, Homo sapiens
  Residue 102-116 RhCe
PSGKVVITLFSIRLA

SEQ ID NO: 222 - protein, 15 aa, Homo sapiens
  Residue 107-121 RhCe
VITLFSIRLATMSAM

SEQ ID NO: 223 - protein, 15 aa, Homo sapiens
  Residue 112-126 RhCe
SIRLATMSAMSVLIS

SEQ ID NO: 224 - protein, 15 aa, Homo sapiens
  Residue 117-131 RhCe
TMSAMSVLISAGAVL

SEQ ID NO: 225 - protein, 15 aa, Homo sapiens
  Residue 122-136 RhCe
SVLISAGAVLGKVNL

SEQ ID NO: 226 - protein, 15 aa, Homo sapiens
  Residue 127-141 RhCe
AGAVLGKVNLAQLVV

SEQ ID NO: 227 - protein, 15 aa, Homo sapiens
  Residue 132-146 RhCe
GKVNLAQLVVMVLVE

SEQ ID NO: 228 - protein, 15 aa, Homo sapiens
  Residue 142-156 RhCe
MVLVEVTALGTLRMV

SEQ ID NO: 229 - protein, 15 aa, Homo sapiens
  Residue 147-161 RhCe
VTALGTLRMVISNIF

SEQ ID NO: 230 - protein, 15 aa, Homo sapiens
  Residue 152 - 166 RhCe
TLRMVISNIFNTDYH

SEQ ID NO: 231 - protein, 15 aa, Homo sapiens
  Residue 157-171 RhCe
ISNIFNTDYHMNLRH

SEQ ID NO: 232 - protein, 15 aa, Homo sapiens
  Residue 162-176 RhCe
NTDYHMNLRHIYVFA

SEQ ID NO: 233 - protein, 15 aa, Homo sapiens
  Residue 167-181 RhCe
MNLRHIYVFAAYFGL

SEQ ID NO: 234 - protein, 15 aa, Homo sapiens
  Residue 172-186 RhCe
IYVFAAYFGLTVAWC

SEQ ID NO: 235 - protein, 15 aa, Homo sapiens
  Residue 177-191 RhCe
AYFGLTVAWCLPKPL

SEQ ID NO: 236 - protein, 15 aa, Homo sapiens
  Residue 182-196 RhCe
TVAWCLPKPLPKGTE

SEQ ID NO: 237 - protein, 15 aa, Homo sapiens
  Residue 187-201 RhCe
LPKPLKEGTEDKDQR

SEQ ID NO: 238 - protein, 15 aa, Homo sapiens
  Residue 192-206 RhCe
PKGTEDNDQRATIPS

SEQ ID NO: 239 - protein, 15 aa, Homo sapiens
  Residue 197-211 RhCe
DNDQRATIPSLSAML

SEQ ID NO: 240 - protein, 15 aa, Homo sapiens
  Residue 202-216 RhCe
ATIPSLSAMLGALFL

SEQ ID NO: 241 - protein, 15 aa, Homo sapiens
  Residue 207-221 RhCe
LSAMLGALFLWMFWP

SEQ ID NO: 242 - protein, 15 aa, Homo sapiens
  Residue 212-226 RhCe
GALFLWMFWPSVNSA

SEQ ID NO: 243 - protein, 15 aa, Homo sapiens
  Residue 217-231 RhCe
WMFWPSVNSALLRSP

SEQ ID NO: 244 - protein, 15 aa, Homo sapiens
  Residue 222-236 RhCe
SVNSALLRSPIQRKN

SEQ ID NO: 245 - protein, 15 aa, Homo sapiens
  Residue 227-241 RhCe
LLRSPIQRKNAMFNT

SEQ ID NO: 246 - protein, 15 aa, Homo sapiens
  Residue 232-246 RhCe
IQRKNAMFNTYYALA

SEQ ID NO: 247 - protein, 15 aa, Homo sapiens
  Residue 237-251 RhCe
AMFNTYYALAVSVVT

SEQ ID NO: 248 - protein, 15 aa, Homo sapiens
  Residue 242-256 RhCe
YYAVAVSVVTAISGS

SEQ ID NO: 249 - protein, 15 aa, Homo sapiens
  Residue 252-266 RhCe
AISGSSLAHPQRKIS

SEQ ID NO: 250 - protein, 15 aa, Homo sapiens
  Residue 257-271 RhCe
SLAHPQRKISMTYVH

SEQ ID NO: 251 - protein, 15 aa, Homo sapiens
  Residue 262-276 RhCe
QRKISMTYVHSAVLA

SEQ ID NO: 252 - protein, 15 aa, Homo sapiens
  Residue 267-281 RhCe
MTYVHSAVLAGGVAV

SEQ ID NO: 253 - protein, 15 aa, Homo sapiens
  Residue 272-286 RhCe
SAVLAGGVAVGTSCH

SEQ ID NO: 254 - protein, 15 aa, Homo sapiens
  Residue 282-296 RhCe
GTSCHLIPSPWLAMV

SEQ ID NO: 255 - protein, 15 aa, Homo sapiens
  Residue 292-306 RhCe
WLAMVLGLVAGLISI

SEQ ID NO: 256 - protein, 15 aa, Homo sapiens
  Residue 297-311 RhCe
LGLVAGLISIGGAKY

SEQ ID NO: 257 - protein, 15 aa, Homo sapiens
  Residue 302-316 RhCe
GLISIGGAKCLPVCC

SEQ ID NO: 258 - protein, 15 aa, Homo sapiens
  Residue 307-321 RhCe
GGAKCLPVCCNRVLG

SEQ ID NO: 259 - protein, 15 aa, Homo sapiens
  Residue 312-326 RhCe
LPVCCNRVLGIHHIS

SEQ ID NO: 260 - protein, 15 aa, Homo sapiens
  Residue 317-331 RhCe
NRVLGIHHISVMHSI

SEQ ID NO: 261 - protein, 15 aa, Homo sapiens
  Residue 322-336 RhCe
IHHISVMHSIFSLLG

SEQ ID NO: 262 - protein, 15 aa, Homo sapiens
  Residue 327-341 RhCe
IMHSIFSLLGLLGEI

SEQ ID NO: 263 - protein, 15 aa, Homo sapiens
  Residue 332-346 RhCe
FSLLGLLGEITYIVL

SEQ ID NO: 264 - protein, 15 aa, Homo sapiens
  Residue 337-351 RhCe
LLGEITYIVLLVLHT

SEQ ID NO: 265 - protein, 15 aa, Homo sapiens
  Residue 342-356 RhCe
TYIVLLVLHTVWNGN

SEQ ID NO: 266 - protein, 15 aa, Homo sapiens
  Residue 347-361 RhCe
LVLHTVWNGNGMIGF

SEQ ID NO: 267 - protein, 15 aa, Homo sapiens
  Residue 352-366 RhCe
VWNGNGMIGFQVLLS

SEQ ID NO: 268 - protein, 15 aa, Homo sapiens
  Residue 362-376 RhCe
QVLLSIGELSLAIVI

SEQ ID NO: 269 - protein, 15 aa, Homo sapiens
  Residue 372-386 RhCe
LAIVIALTSGLLTGL

SEQ ID NO: 270 - protein, 15 aa, Homo sapiens
  Residue 382-396 RhCe
LLTGLLLNLKIWKAP

SEQ ID NO: 271 - protein, 15 aa, Homo sapiens
  Residue 387-401 RhCe
LLNLKIWKAPHVAKY

SEQ ID NO: 272 - protein, 15 aa, Homo sapiens
  Residue 392-406 RhCe
IWKAPHVAKYFDDQV

SEQ ID NO: 273 - protein, 15 aa, Homo sapiens
  Residue 397-411 RhCe
HVAKYFDDQVFWKFP

SEQ ID NO: 274 - protein, 15 aa, Homo sapiens
  Residue 402-416 RhCe
FDDQVFWKFPHLAVG

SEQ ID NO: 275 - protein, 15 aa, Homo sapiens
  Residue 403-417 RhCe
DDQVFWKFPHLAVGF

SEQ ID NO: 276 - protein, 15 aa, Homo sapiens
  Residues 2-16 RhCE
SSKYPRSVRRCLPLC

SEQ ID NO: 277 - protein, 15 aa, Homo sapiens
  Residues 12-26 RhCE
CLPLCALTLEAALIL

SEQ ID NO: 278 - protein, 15 aa, Homo sapiens
  Residue 22-36 RhCE
AALILLFYFFTHYDA

SEQ ID NO: 279 - protein, 15 aa, Homo sapiens
  Residues 32-46 RhCE
THYDASLEDQKGLVA

SEQ ID NO: 280 - protein, 15 aa, Homo sapiens
  Residue 42-56 RhCE
KGLVASYQVGQDLTV

SEQ ID NO: 281 - protein, 15 aa, Homo sapiens
  Residue 52-66 RhCE
QDLTVMAAIGLGFLT

SEQ ID NO: 282 - protein, 15 aa, Homo sapiens
  Residue 57-71 RhCE
MAAIGLGFLTSNSRR

SEQ ID NO: 283 - protein, 15 aa, Homo sapiens
  Residue 62-76 RhCE
LGFLTSSFRRHSWSS

SEQ ID NO: 284 - protein, 15 aa, Homo sapiens
  Residue 67-81 RhCE
SSFRRHSWSSVAFNL

SEQ ID NO: 285 - protein, 15 aa, Homo sapiens
  Residue 72-86 RhCE
HSWSSVAFNLFMLAL

SEQ ID NO: 286 - protein, 15 aa, Homo sapiens
  Residue 82-96 RhCE
FMLALGVQWAILLDG

SEQ ID NO: 287 - protein, 15 aa, Homo sapiens
  Residue 92-106 RhCE
ILLDGFLSQFPSGKV

SEQ ID NO: 288 - protein, 15 aa, Homo sapiens
  Residue 97-111 RhCE
FLSQFPSGKVVITLF

SEQ ID NO: 289 - protein, 15 aa, Homo sapiens
  Residue 102-116 RhCE
PSGKVVITLFSIRLA

SEQ ID NO: 290 - protein, 15 aa, Homo sapiens
  Residue 107-121 RhCE
VITLFSIRLATMSAM

SEQ ID NO: 291 - protein, 15 aa, Homo sapiens
  Residue 112-126 RhCE
SIRLATMSAMSVLIS

SEQ ID NO: 292 - protein, 15 aa, Homo sapiens
  Residue 117-131 RhCE
TMSAMSVLISAGAVL

SEQ ID NO: 293 - protein, 15 aa, Homo sapiens
  Residue 122-136 RhCE
SVLISAGAVLGKVNL

SEQ ID NO: 294 - protein, 15 aa, Homo sapiens
  Residue 127-141 RhCE
AGAVLGKVNLAQLVV

SEQ ID NO: 295 - protein, 15 aa, Homo sapiens
  Residue 132-146 RhCE
GKVNLAQLVVMVLVE

SEQ ID NO: 296 - protein, 15 aa, Homo sapiens
  Residue 142-156 RhCE
MVLVEVTALGTLRMV

SEQ ID NO: 297 - protein, 15 aa, Homo sapiens
  Residue 147-161 RhCE
VTALGTLRMVISNIF

SEQ ID NO: 298 - protein, 15 aa, Homo sapiens
  Residue 152-166 RhCE
TLRMVISNIFNTDYH

SEQ ID NO: 299 - protein, 15 aa, Homo sapiens
  Residue 157-171 RhCE
ISNIFNTDYHMNLRH

SEQ ID NO: 300 - protein, 15 aa, Homo sapiens
  Residue 162-176 RhCE
NTDYHMNLRHIYVFA

SEQ ID NO: 301 - protein, 15 aa, Homo sapiens
  Residue 167-181 RhCE
MNLRHIYVFAAYFGL

SEQ ID NO: 302 - protein, 15 aa, Homo sapiens
  Residue 172-186 RhCE
IYVFAAYFGLTVAWC

SEQ ID NO: 303 - protein, 15 aa, Homo sapiens
  Residue 177-191 RhCE
AYFGLTVAWCLPKPL

SEQ ID NO: 304 - protein, 15 aa, Homo sapiens
  Residue 182-196 RhCE
TVAWCLPKPLPKGTE

SEQ ID NO: 305 - protein, 15 aa, Homo sapiens
  Residue 187-201 RhCE
LPKPLKEGTEDKDQR

SEQ ID NO: 306 - protein, 15 aa, Homo sapiens
  Residue 192-206 RhCE
PKGTEDNDQRATIPS

SEQ ID NO: 307 - protein, 15 aa, Homo sapiens
  Residue 197-211 RhCE
DNDQRATIPSLSAML

SEQ ID NO: 308 - protein, 15 aa, Homo sapiens
  Residue 202-216 RhCE
ATIPSLSAMLGALFL

SEQ ID NO: 309 - protein, 15 aa, Homo sapiens
  Residue 207-221 RhCE
LSAMLGALFLWMFWP

SEQ ID NO: 310 - protein, 15 aa, Homo sapiens
  Residue 212-226 RhCE
GALFLWMFWPSVNSP

SEQ ID NO: 311 - protein, 15 aa, Homo sapiens
  Residue 217-231 RhCE
WMFWPSVNSPLLRSP

SEQ ID NO: 312 - protein, 15 aa, Homo sapiens
  Residue 222-236 RhCE
SVNSPLLRSPIERKN

SEQ ID NO: 313 - protein, 15 aa, Homo sapiens
  Residue 227-241 RhCE
LLRSPIQRKNAMFNT

SEQ ID NO: 314 - protein, 15 aa, Homo sapiens
  Residue 232-246 RhCE
IQRKNAMFNTYYALA

SEQ ID NO: 315 - protein, 15 aa, Homo sapiens
  Residue 237-251 RhCE
AMFNTYYALAVSVVT

SEQ ID NO: 316 - protein, 15 aa, Homo sapiens
  Residue 242-256 RhCE
YYAVAVSVVTAISGS

SEQ ID NO: 317 - protein, 15 aa, Homo sapiens
  Residue 252-266 RhCE
AISGSSLAHPQRKIS

SEQ ID NO: 318 - protein, 15 aa, Homo sapiens
  Residue 257-271 RhCE
SLAHPQRKISMTYVH

SEQ ID NO: 319 - protein, 15 aa, Homo sapiens
  Residue 262-276 RhCE
QRKISMTYVHSAVLA

SEQ ID NO: 320 - protein, 15 aa, Homo sapiens
  Residue 267-281 RhCE
MTYVHSAVLAGGVAV

SEQ ID NO: 321 - protein, 15 aa, Homo sapiens
  Residue 272-286 RhCE
SAVLAGGVAVGTSCH

SEQ ID NO: 322 - protein, 15 aa, Homo sapiens
  Residue 282-296 RhCE
GTSCHLIPSPWLAMV

SEQ ID NO: 323 - protein, 15 aa, Homo sapiens
  Residue 292-306 RhCE
WLAMVLGLVAGLISI

SEQ ID NO: 324 - protein, 15 aa, Homo sapiens
  Residue 297-311 RhCE
LGLVAGLISIGGAKY

SEQ ID NO: 325 - protein, 15 aa, Homo sapiens
  Residue 302-316 RhCE
GLISIGGAKCLPVCC

SEQ ID NO: 326 - protein, 15 aa, Homo sapiens
  Residue 307-321 RhCE
GGAKCLPVCCNRVLG

SEQ ID NO: 327 - protein, 15 aa, Homo sapiens
  Residue 312-326 RhCE
LPVCCNRVLGIHHIS

SEQ ID NO: 328 - protein, 15 aa, Homo sapiens
  Residue 317-331 RhCE
NRVLGIHHISVMHSI

SEQ ID NO: 329 - protein, 15 aa, Homo sapiens
  Residue 322-336 RhCE
IHHISVMHSIFSLLG

SEQ ID NO: 330 - protein, 15 aa, Homo sapiens
  Residue 327-341 RhCE
IMHSIFSLLGLLGEI

SEQ ID NO: 331 - protein, 15 aa, Homo sapiens
  Residue 332-346 RhCE
FSLLGLLGEITYIVL

SEQ ID NO: 332 - protein, 15 aa, Homo sapiens
  Residue 337-351 RhCE
LLGEITYIVLLVLHT

SEQ ID NO: 333 - protein, 15 aa, Homo sapiens
  Residue 342-356 RhCE
TYIVLLVLHTVWNGN

SEQ ID NO: 334 - protein, 15 aa, Homo sapiens
  Residue 347-361 RhCE
LVLHTVWNGNGMIGF

SEQ ID NO: 335 - protein, 15 aa, Homo sapiens
  Residue 352-366 RhCE
VWNGNGMIGFQVLLS

SEQ ID NO: 336 - protein, 15 aa, Homo sapiens
  Residue 362-376 RhCE
QVLLSIGELSLAIVI

SEQ ID NO: 337 - protein, 15 aa, Homo sapiens
  Residue 372-386 RhCE
LAIVIALTSGLLTGL

SEQ ID NO: 338 - protein, 15 aa, Homo sapiens
  Residue 382-396 RhCE
LLTGLLLNLKIWKAP

SEQ ID NO: 339 - protein, 15 aa, Homo sapiens
  Residue 387-401 RhCE
LLNLKIWKAPHVAKY

SEQ ID NO: 340 - protein, 15 aa, Homo sapiens
  Residue 392-406 RhCE
IWKAPHVAKYFDDQV

SEQ ID NO: 341 - protein, 15 aa, Homo sapiens
  Residue 397-411 RhCE
HVAKYFDDQVFWKFP

SEQ ID NO: 342 - protein, 15 aa, Homo sapiens
  Residue 402-416 RhCE
FDDQVFWKFPHLAVG

SEQ ID NO: 343 - protein, 15 aa, Homo sapiens
  Residue 403-417 RhCE
DDQVFWKFPHLAVGF

SEQ ID NO: 344 - protein, 15 aa, Homo sapiens
  Residue 1-15 Rh50GP
MRFTFPLMAIVLEIA

SEQ ID NO: 345 - protein, 15 aa, Homo sapiens
  Residue 11-25 Rh50GP
VLEIAMIVLFGLFVE

SEQ ID NO: 346 - protein, 15 aa, Homo sapiens
  Residue 21-35 Rh50GP
GLFVEYETDQTVLEQ

SEQ ID NO: 347 - protein, 15 aa, Homo sapiens
  Residue 31-45 Rh50GP
TVLEQLNITKPTDMG

SEQ ID NO: 348 - protein, 15 aa, Homo sapiens
  Residue 41-55 Rh50GP
PTDMGIFFELYPLFQ

SEQ ID NO: 349 - protein, 15 aa, Homo sapiens
  Residue 51-65 Rh50GP
YPLFQDVHVMIFVGF

SEQ ID NO: 350 - protein, 15 aa, Homo sapiens
  Residue 61-75 Rh50GP
IFVGFGFLMTFLKKY

SEQ ID NO: 351 - protein, 15 aa, Homo sapiens
  Residue 71-85 Rh50GP
FLKKYGFSSVGINLL

SEQ ID NO: 352 - protein, 15 aa, Homo sapiens
  Residue 81-95 Rh50 GP
GINLLVAALGLQWGT

SEQ ID NO: 353 - protein, 15 aa, Homo sapiens
  Residue 91-105 Rh50GP
LQWGTIVQGILQSQG

SEQ ID NO: 354 - protein, 15 aa, Homo sapiens
  Residue 101-115 Rh50GP
LQSQGQKFNIGIKNM

SEQ ID NO: 355 - protein, 15 aa, Homo sapiens
  Residue 111-125 Rh50GP
GIKNMINADFSAATV

SEQ ID NO: 356 - protein, 15 aa, Homo sapiens
  Residue 121-135 Rh50GP
SAATVLISFGAVLGK

SEQ ID NO: 357 - protein, 15 aa, Homo sapiens
  Residue 131-145 Rh50GP
AVLGKTSPTQMLIMT

SEQ ID NO: 358 - protein, 15 aa, Homo sapiens
  Residue 141-155 Rh50GP
MLIMTILEIVFFAHN

SEQ ID NO: 359 - protein, 15 aa, Homo sapiens
  Residue 151-165 Rh50GP
FFAHNEYLVSEIFKA

SEQ ID NO: 360 - protein, 15 aa, Homo sapiens
  Residue 161-175 Rh50GP
EIFKASDIGASMTIH

SEQ ID NO: 361 - protein, 15 aa, Homo sapiens
  Residue 171-185 Rh50GP
SMTIHAFGAYFGLAV

SEQ ID NO: 362 - protein, 15 aa, Homo sapiens
  Residue 181-195 Rh50GP
FGLAVAGILYRSGLR

SEQ ID NO: 363 - protein, 15 aa, Homo sapiens
  Residue 191-205 Rh50GP
RSGLRKGHENEESAY

SEQ ID NO: 364 - protein, 15 aa, Homo sapiens
  Residue 201-215 Rh50GP
EESAYYSDLFAMIGT

SEQ ID NO: 365 - protein, 15 aa, Homo sapiens
  Residue 211-225 Rh50GP
AMIGTLFLWMFWPSF

SEQ ID NO: 366 - protein, 15 aa, Homo sapiens
  Residue 221-235 Rh50GP
FWPSFNSAIAEPGDK

SEQ ID NO: 367 - protein, 15 aa, Homo sapiens
  Residue 231-245 Rh50GP
EPGDKQCRAIVDTYF

SEQ ID NO: 368 - protein, 15 aa, Homo sapiens
  Residue 241-255 Rh50GP
VDTYFSLAACVLTAF

SEQ ID NO: 369 - protein, 15 aa, Homo sapiens
  Residue 251-265 Rh50GP
VLTAFAFSSLVEHRG

SEQ ID NO: 370 - protein, 15 aa, Homo sapiens
  Residue 261-275 Rh50GP
VEHRGKLNMVHIQNA

SEQ ID NO: 371 - protein, 15 aa, Homo sapiens
  Residue 271-285 Rh50GP
HIQNATLAGGVAVGT

SEQ ID NO: 372 - protein, 15 aa, Homo sapiens
  Residue 281-295 Rh50GP
VAVGTCADMAIHPFG

SEQ ID NO: 373 - protein, 15 aa, Homo sapiens
  Residue 291-305 Rh50GP
IHPFGSMIIGSIAGM

SEQ ID NO: 374 - protein, 15 aa, Homo sapiens
  Residue 301-315 Rh50GP
SIAGMVSVLGYKFLT

SEQ ID NO: 375 - protein, 15 aa, Homo sapiens
  Residue 311-325 Rh50GP
YKFLTPLFTTKLRIH

SEQ ID NO: 376 - protein, 15 aa, Homo sapiens
  Residue 321-335 Rh50GP
KLRIHDTCGVHNLHG

SEQ ID NO: 377 - protein, 15 aa, Homo sapiens
  Residue 331-345 Rh50GP
HNLHGLPGVVGGLAG

SEQ ID NO: 378 - protein, 15 aa, Homo sapiens
  Residue 341-355 Rh50GP
GGLAGIVAVAMGASN

SEQ ID NO: 379 - protein, 15 aa, Homo sapiens
  Residue 351-365 Rh50GP
MGASNTSMAMQAAAL

SEQ ID NO: 380 - protein, 15 aa, Homo sapiens
  Residue 361-375 Rh50GP
QAAALGSSIGTAVVG

SEQ ID NO: 381 - protein, 15 aa, Homo sapiens
  Residue 371-385 Rh50GP
TAVVGGLMTGLILKL

SEQ ID NO: 382 - protein, 15 aa, Homo sapiens
  Residue 381-395 Rh50GP
LILKLPLWGQPSDQN

SEQ ID NO: 383 - protein, 15 aa, Homo sapiens
  Residue 391-405 Rh50GP
PSDQNCYDDSVYWKV

SEQ ID NO: 384 - protein, 417 aa, Homo sapiens
  RhCE
MSSKYPRSVRRCLPLCALTLEAALILLFYFFTHYDASLEDQKGLVASYQVGQDLTVMAAIGLGFLTSSFRRHSWSSVAFNLFMLALGVQWAILLDGFLSQFPSGKVVITLFSIRLATMSAMSVLISAGAVLGKVNLAQLVVMVLVEVTALGTLRMVISNIFNTDYHMNLRHFYVFAAYFGLTVAWCLPKPLPKGTEDNDQRATIPSLSAMLGALFLWMFWPSVNSPLLRSPIQRKNAMFNTYYALAVSVVTAISGSSLAHPQRKISMTYVHSAVLAGGVAVGTSCHLIPSPWLAMVLGLVAGLISIGGAKCLPVCCNRVLGIHHISVMHSIFSLLGLLGEITYIVLLVLHTVWNGNGMIGFQVLLSIGELSLAIVIALTSGLLTGLLLNLKIWKAPHVAKYFDDQVFWKFPHLAVGF

SEQ ID NO: 385 - protein, 417 aa, Homo sapiens
  RhCe
MSSKYPRSVRRCLPLCALTLEAALILLFYFFTHYDASLEDQKGLVASYQVGQDLTVMAAIGLGFLTSSFRRHSWSSVAFNLFMLALGVQWAILLDGFLSQFPSGKVVITLFSIRLATMSAMSVLISAGAVLGKVNLAQLVVMVLVEVTALGTLRMVISNIFNTDYHMNLRHFYVFAAYFGLTVAWCLPKPLPKGTEDNDQRATIPSLSAMLGALFLWMFWPSVNSALLRSPIQRKNAMFNTYYALAVSVVTAISGSSLAHPQRKISMTYVHSAVLAGGVAVGTSCHLIPSPWLAMVLGLVAGLISIGGAKCLPVCCNRVLGIHHISVMHSIFSLLGLLGEITYIVLLVLHTVWNGNGMIGFQVLLSIGELSLAIVIALTSGLLTGLLLNLKIWKAPHVAKYFDDQVFWKFPHLAVGF

SEQ ID NO: 386 - protein, 417 aa, Homo sapiens [sequence omitted]

SEQ ID NO: 387 - protein, 417 aa, Homo sapiens
  RhD
MSSKYPRSVRRCLPLWALTLEAALILLFYFFTHYDASLEDQKGLVASYQVGQDLTVMAAIGLGFLTSSFRRHSWSSVAFNLFMLALGVQWAILLDGFLSQFPSGKVVITLFSIRLATMSALSVLISVDAVLGKVNLAQLVVMVLVEVTALGNLRMVISNIFNTDYHMNMMHIYVFAAYFGLSVAWCLPKPLPEGTEDNDQTATIPSLSAMLGALFLWIFWPSFNSALLRSPIERKNAVFNTYYAVAVSVVTAISGSSLAHPQGKISKTYVHSAVLAGGVAVGTSCHLIPSPWLAMVLGLVAGLISVGGAKYLPGCCNRVLGIPHSSIMGYNFSLLGLLGEIIYIVLLVLDTVGAGNGMIGFQVLLSIGELSLAIVIALTSGLLTGLLLNLKIWKAPHEAKYFDDQVFWKFPHLAVGF

SEQ ID NO: 388 - protein, 417 aa, Homo sapiens
  Rhce
MSSKYPRSVRRCLPLWALTLEAALILLFYFFTHYDASLEDQKGLVASYQVGQDLTVMAALGLGFLTSNFRRHSWSSVAFNLFMLALGVQWAILLDGFLSQFPPGKVVITLFSIRLATMSAMSVLISAGAVLGKVNLAQLVVMVLVEVTALGTLRMVISNIFNTDYHMNLRHFYVFAAYFGLTVAWCLPKPLPKGTEDNDQRATIPSLSAMLGALFLWMFWPSVNSALLRSPIQRKNAMFNTYYALAVSVVTAISGSSLAHPQRKISMTYVHSAVLAGGVAVGTSCHLIPSPWLAMVLGLVAGLISIGGAKCLPVCCNRVLGIHHISVMHSIFSLLGLLGEITYIVLLVLHTVWNGNGMIGFQVLLSIGELSLAIVIALTSGLLTGLLLNLKIWKAPHVAKYFDDQVFWKFPHLAVGF

(19) United States
(12) Patent Application Publication
(10) Pub. No.: US 2005/0069544 A1
(43) Pub. Date: Mar. 31, 2005

(54) Pharmaceutical composition and a method of treatment

(76) Inventors: Stanislaw Joseph Urbaniak, Aberdeen (GB); Robert Norman Barker, Inverurie (GB)

Correspondence Address: Licata & Tyrell P.C., 66 East Main Street, Marlton, NJ, 08053, US

(21) Appl. No.: 10/701,682

(22) Filed: Nov. 4, 2003

Related U.S. Application Data
(63) Continuation-in-part of application No. 09/857,097, filed on Jul. 27, 2001.

(30) Foreign Application Priority Data
Dec. 1, 1998 (GB) 9826378.3

(51) Int. Cl. A61K 39/395

(52) U.S. Cl. 424/144.1

(57) ABSTRACT

Pharmaceutical compositions and methods are provided for the prevention of a condition resulting from the alloimmunisation or autoimmunity of a subject or the immunosuppression of a response elicited by alloimmunisation or autoimmunity of a subject by tolerisation. These compositions contain an immunologically effective amount of an epitope from a rhesus protein or a peptide fragment, an immunoreactive analogue or derivative or a cross-reaction sequence thereof.

The present invention relates to the mapping of allo-reactive T-cell epitopes on the rhesus(RhD and RhCc/Ee) proteins and to the use of such epitopes to modulate the corresponding immune responses to these antigens. The present invention relates to compositions and methods for the treatment of diseases or illness relating to red blood cells and platelets, in particular haemolytic disease of the newborn (HDN), autoimmune haemolytic anemia (AIHA)and foetomaternal alloimmune thrombocytopenia (ATP).

Human blood contains a genetically complex rhesus (Rh) blood group system. For example, humans are either RhD positive or negative and this can lead to problems during transfusions or pregnancy when RhD negative individuals are exposed to RhD positive blood and become immunised to produce anti-D.

The most important allele in the RhD blood group system is the D antigen. The RhD antigen is carried by the RhD protein which is a transmembrane protein consisting of 417 amino acids with 12 putative transmembrane domains and 6 extracellular loops.

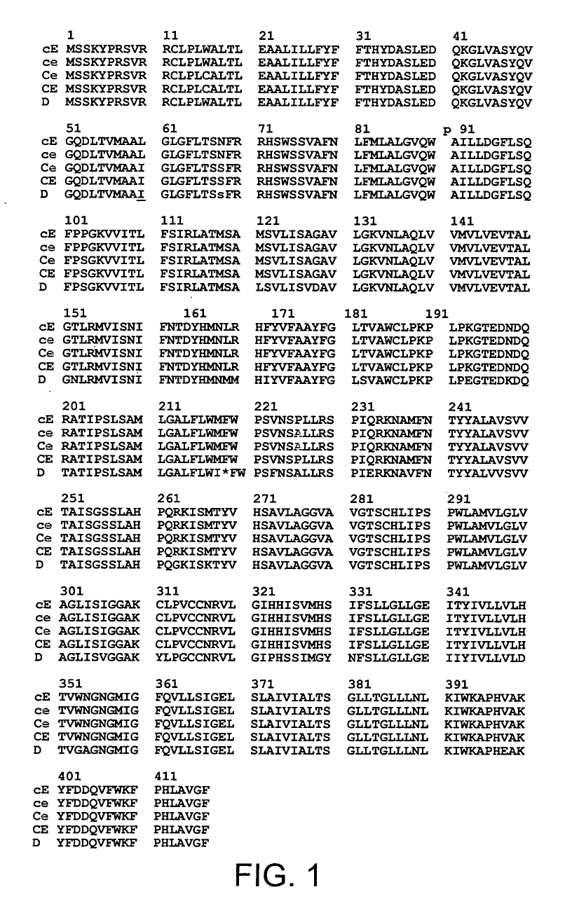

The full amino acid sequence of the RhD, RhcE, Rhce, RhCe and RhCE polypeptide and the differences in sequence for polypeptides is shown in FIG. 1 hereinafter (Reference: The Blood Group Antigen Facts Book, p94, Editors; M E Reid & C Lomas-Francis, Academic Press London).

The complexity of the blood system can cause problems during pregnancy when a woman who is RhD negative is carrying a RhD positive foetus, as the woman is at risk of being immunized by the RhD positive blood cells of her own baby. This immunisation can take place during situations when the mother's and baby's blood can become mixed, for example during amniocentesis, antepartum haemorrhage but mainly at parturition.

Once the mother's immune system has been exposed to RhD positive blood cells, she may produce anti-D antibodies, which can cross the placenta and cause Rh haemolytic disease in any subsequent RhD positive pregnancies. Such haemolytic disease can be fatal for the neonate.

Currently, purified anti-D immunoglobulin is injected whenever a mother is exposed to foetal RhD positive red blood cells, which may occur during e.g., amniocentesis, antepartum haemorrhage but mainly at parturition. About 17% of Caucasian women are RhD negative so that most industrialized countries have RhD prevention programmes wherein all RhD negative women receive prophylaxis with anti-D immunoglobulin at delivery or in association with the other high risk events alluded to above. Further in many countries, routine antepartum prophylaxis to minimize the incidence of Rh haemolytic disease is practised.

There are a number of problems with this approach. Firstly, efficacy is never entirely complete since events can be missed or undeclared, or a foetal haemorrhage can be larger than the anti-D can neutralize. Secondly, current anti-D immunoglobulin comes from deliberately immunised donors, which puts volunteers, often male (paid or not) at some small risk. In addition it takes at least 12 months to accredit the donors, during which time their blood products are not available. For these reasons there is a worldwide shortage of anti-D immunoglobulin. Finally, there are also concerns about the safety of recipients who may be exposed to transfusion transmitted infections such as by inadvertent infection with agents, for example variant Creutzfeld-Jacob Disease (vCJD) for which there is no satisfactory test.

Other groups that can be at risk from alloimmunisation are those who are regular recipients of blood products, for example those suffering from hemological malignant disease, sickle cell disease or thalassaemia.

The RhD, RhcE, Rhce, RhCe, RhCE proteins are also characteristically the target of autoantibodies produced by most patients with warm type AIHA. IgG autoantibody coating of erythrocytes in vivo can result in erythrocyte destruction, which manifests as a decrease in erythrocyte count and haemoglobin levels. Non-specific symptoms include jaundice, malaise and dizziness. As the disease progresses patients become increasingly lethargic and episodes of rapid hemolysis can be life threatening. Clearance of IgG coated erythrocytes occurs through Fc Receptors (FcR) on phagocytes primarily in the spleen that recognise either monomeric IgG (via FcRI) or dimeric IgG (via FcRII or FcRIII). The process can be augmented by fixation of complement components such as C3b and C4b.

The incidence of warm type AIHA is approximately 1:75,000-80,000 of the population with the peak incidence occurring over the age of sixty. More recent studies have also demonstrated that the incidence of primary warm type AIHA rises with age: up to around forty years of age the incidence of warm type AIHA is less than 1 in 100,000, this however increases to around 1 in 8,000 at the age of eighty.

Primary warm type AIHA patients typically suffer chronic disease. The first line treatment is steroids such as prednisolone. More severe cases will be treated with cytotoxic drugs such as azothiaprine. Both these approaches have serious side effects and leave the patient open to secondary infection.

Unresponsive patients can undergo splenectomy thereby removing a major site of erythrocyte clearance, but this surgery is not without risk in anaemic elderly patients, and the benefits may be temporary.

An object of the present invention is to overcome the disadvantages of the prior art.

According to one aspect of the present invention there is provided a pharmaceutical composition for the prevention of a condition which results from the alloimmunisation or autoimmunity of a subject or the immunosuppression of a response elicited by alloimmunisation or autoimmunity of a subject by tolerisation, said composition comprising an immunologically effective amount of an epitope from a rhesus protein or a peptide fragment, an immunoreactive analogue or derivative or a cross-reaction sequence thereof.

The skilled person will be aware that any T-cell that responds to a given peptide can also respond in a similar way to other peptides containing substitutions in residues that are not critical for MHC binding or T-cell receptor recognition, and even to certain peptides that are substituted in critical residues.

It has been found that a subject can be prevented from acquiring a condition, which results from alloimmunisation or autoimmunity, or the condition once it has been obtained can be managed by the administration of immunologically effective epitopes of the protein which has resulted in the alloimmunisation or autoimmunity.

Tolerisation is a non-invasive method, which involves providing relatively small amounts of a peptide or protein to a patient generally through mucosal tissue. The patient's immune system then over a period of time becomes tolerant to the peptide or protein and, therefore, does not consider the protein or peptide foreign. Accordingly, no effector immune response is raised.

A series of peptides has been constructed in the present invention based on the RhD protein each being 15 amino acids (AA) long (Table 1), and tested in vitro against T-lymphocytes from normal individuals, donors who have been alloimmunised to produce anti-D, and patients with warm type autoimmune haemolytic anaemia.

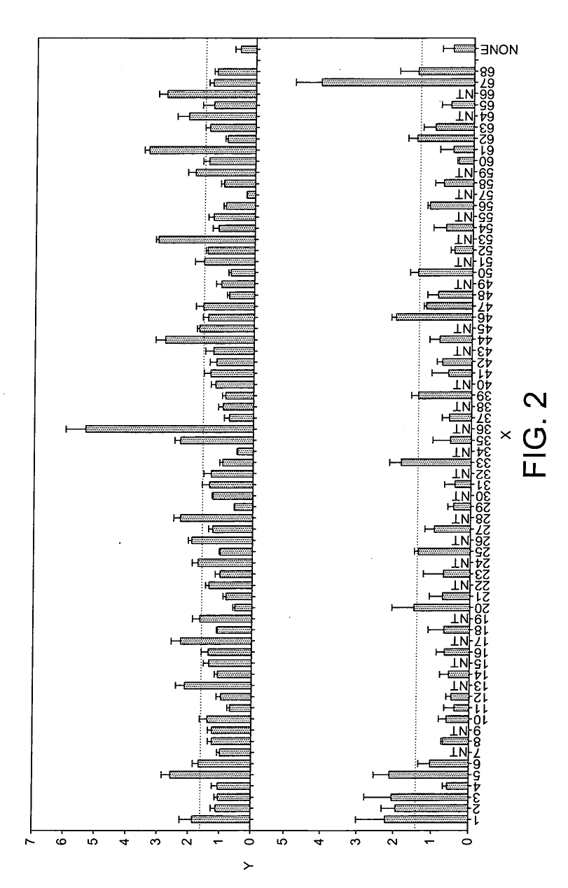

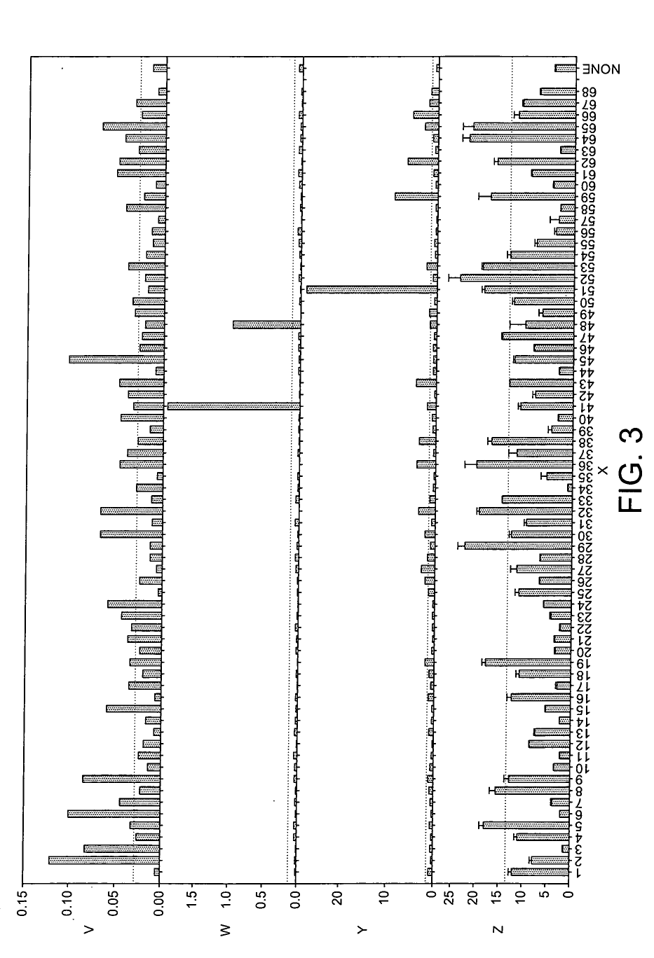

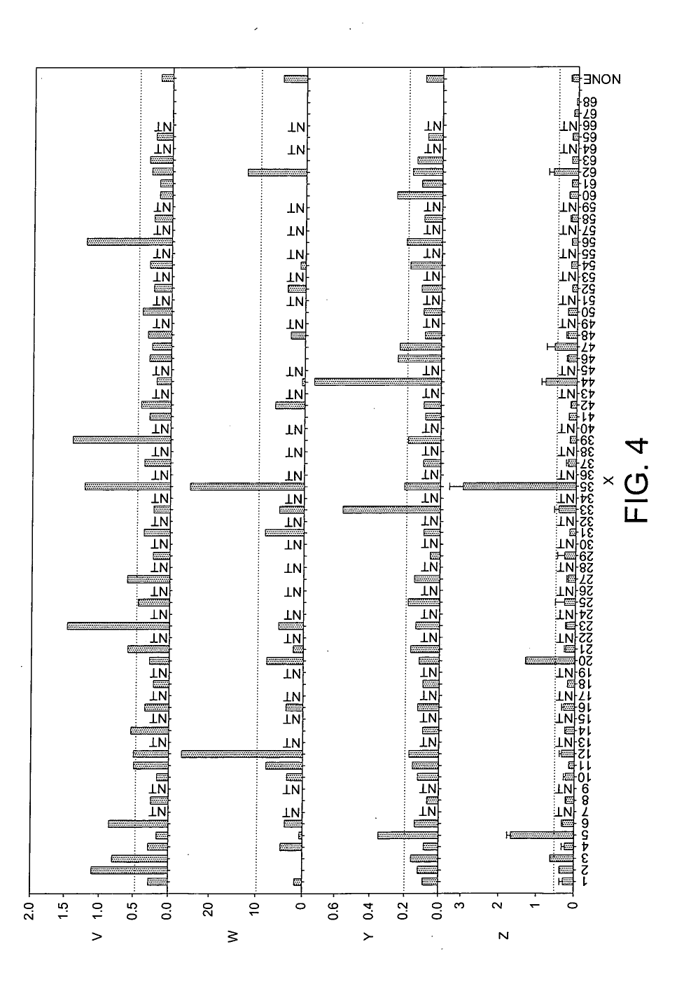

The epitopes, which stimulate a response in donors that have been alloimmunised with RhD protein and AIHA patients, can be different even when the responses were restricted by the same MHC class II element (FIG. 2). Furthermore, the Th-cell subset and cytokine profile secreted in alloreactive and autoreactive responses can also differ (see FIG. 3 and FIG. 4 respectively). The in vitro production of cytokines representative of Th1 (IFN-γ), Th2 (IL-4), Tr1 (IL-10) and Th3 (TGF-β1) responses was measured by cell ELISA to determine if there was bias in the Th-cell subsets stimulated with RhD peptides. Th-cell responses from both groups to stimulatory peptides were dominated by IFN-γ production. Compared to AIHA patients, Th-cells from alloimmunised donors were more likely to produce IL-4, but the amounts were still relatively low.

Significant IL-10 was produced by Th-cells from both alloimmunised donors and AIHA patients. Alloimmunised donors produced TGF-β1 in response to Rh peptide stimulation in vitro but TGF-β1 was rarely produced by Th-cells from AIHA patients.

These results demonstrate that the T-helper cell responses to the RhD protein as an alloantigen are mediated by both Th1 and Th2 subsets, whilst the autoimmune response to the protein is very strongly Th1 biased. Although there is evidence of IL-10 regulatory cell activity in both allo and autoimmune responses, this is not accompanied by TGF-β1 responses in AIHA patients. Furthermore, the T-cell specificity is different in alloimmune and autoimmune responses and is consistent with the view that the autoimmune response is directed against epitopes that are normally poorly processed and presented.

Conveniently, the rhesus protein is selected from RhD, RhcE, Rhce, RhCe or RhCE protein.

These determine the main Rh-specific antigens found on the surface of a red blood cell. The Rh system consists of five different isoforms (C, D, E, c and e), the products of three closely linked loci.

The helper T-cell epitopes on the RhD protein have been mapped. The characterization of a helper epitope that is targeted in most alloimmunised donors and the identification of correlations between HLA-DR type and particular dominant epitopes opens the way for the evaluation of peptide immunotherapy as a novel way to regulate the immune response to RhD and to prevent Rh haemolytic disease, anti-D related transfusion problems, and autoimmune responses to the Rh proteins.

Currently, anti-D which is given to pregnant women during significant events in pregnancy may be considered as a passive form of immunotherapy because it has the effect of blocking the effects of immune events on a temporary basis.

The replacement of passive with active peptide immunotherapy in RhD negative women is an attractive option since safe synthetic tolerogens can be developed and given before pregnancy thus avoiding foetal exposure. Suppression throughout pregnancy could mean that only one administration was necessary, considerably simplifying management of RhD negative women and, for the first time, reversing rather than preventing alloimmunisation by administration of tolerogenic peptides to individuals who have already produced anti-D with the objective of “switching-off” the immune response to RhD.

The replacement of steroids and chemotherapy in AIHA patients with a tolerising peptide reverses established autoimmune responses. Furthermore, the risk of subsequent infection resulting from the use of non-specific immuno-suppressive therapy would be alleviated.

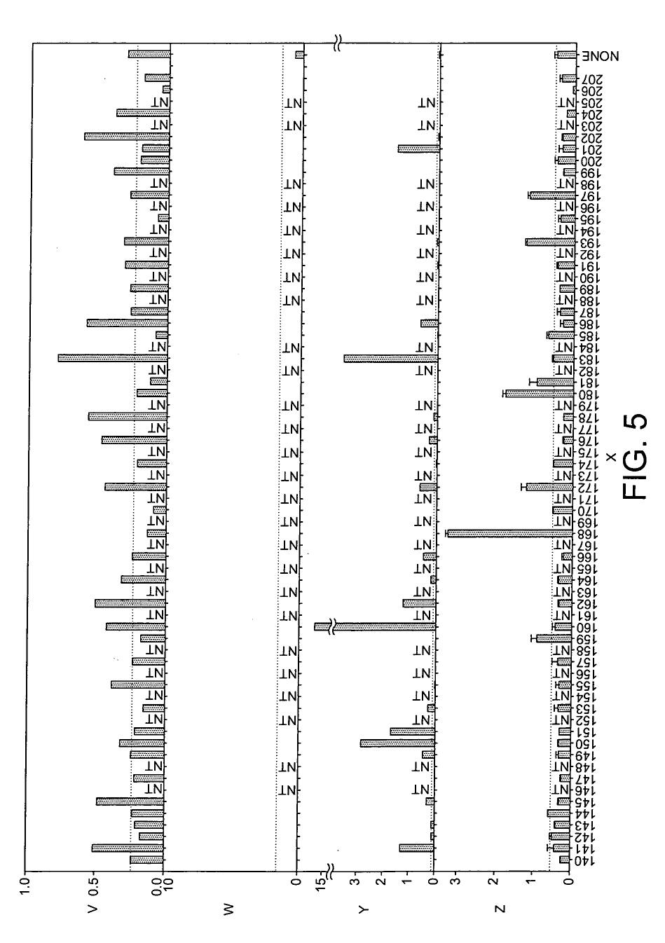

Tolerogenic peptides to other Rh antigens, as determined by methods of the present invention, would be of equivalent value in preventing, or modifying the production of alloantibodies by the respective antigens, including (but not exclusively) RhD, RhcE, Rhce, RhCe or RhCE and Rh50 (peptide amino acid sequences are shown in Tables 1 to 6, respectively) in autoimmune haemolytic anaemia. An illustrative example of autoimmune responses to Rhce, as determined by methods set out in Example 2, as shown in FIG. 5.

Accordingly the categories of individual (which is in no way limiting to the scope of invention) in whom prior immunization would be considered beneficial are as follows:—
(1) all women during their child bearing years; and
(2) regular recipients of blood products who might be exposed to blood transfusion, for example, as a result of haemological malignant disease, sickle cell disease and thalassaemia.

A pharmaceutical composition according to the present invention can be given to expectant mothers with RhD negative blood and a RhD positive child in this respect, which would result in reducing the likelihood of the mother not producing an immune response when the foetus's blood comes into contact with her own immune system. In this connection, there is a reduced likelihood that any subsequent baby that is RhD positive would suffer from haemolytic disease.

The use of synthetic peptides in accordance with the present invention removes concerns about viral infection being transmitted either by anti-D immunoglobulin used for passive immunotherapy or by red blood cells given to volunteer recipients. The time consuming and expensive procedures required to validate accredited donors and donations are also important considerations.

In addition, by use of these compositions volunteers, who are often RhD negative men, can avoid the usual injection of red blood cells when they are deliberately immunised for the production of anti-D immunoglobulin.

If the immune system of a RhD negative mother has already been in contact with the blood from an RhD positive baby, a composition according to the present invention can be used during subsequent pregnancies with an RhD positive baby to reduce the likelihood of the baby suffering from RhD haemolytic disease.

In addition, a composition according to the present invention can be given to patients who have accidentally been given an RhD positive blood transfusion when they are RhD negative. In this connection, the availability of such a composition reduces the need for very large doses of anti-D immunoglobulin to be given to prevent alloimmunisation.

With regard to the sequence listing of RhD, it has been found that at position 218 Ile can be replaced by Met. Met is the amino acid more commonly found at this position.

Preferably, the epitope is selected from at least one of SEQ ID numbers 1 to 383 herein set forth.

It should be noted that SEQ ID Nos:1 to 71 are epitopes from RhD protein, the full sequence of which is SEQ ID No: 387 SEQ ID Nos: 72 to 139 are epitopes from RhcE protein, the full sequence of which is SEQ ID No: 386, SEQ ID Nos: 140 to 207 are epitopes from Rhce protein, the full sequence of which is SEQ ID No: 388, SEQ ID Nos: 208 to 275 are epitopes from RhCe protein, the full sequence of which is SEQ ID No: 385, SEQ ID Nos: 276 to 343 are epitopes from RhCE protein, the full sequence of which is SEQ ID No: 384. Further it should be noted that SEQ ID Nos: 344 to 383 are epitopes from Rh50GP protein.

The aforementioned epitopes are the most common recognised by T-cells of alloimmunised and autoimmune subjects. Induced tolerance to such epitopes would stop an immune response being mounted.

The present composition is suitable for the prevention or management of haemolytic disease of the newborn. Conveniently, when the condition is HDN the epitope is SEQ ID number 1, 2, 3, 4, 5, 6, 7, 8, 9, 10, 11, 12, 13, 14, 15, 16, 17, 18, 19, 20, 21, 22, 24, 25, 26, 27, 28, 29, 33, 34, 35, 36, 37, 40, 41, 43, 44, 45, 46, 47, 48, 49, 50, 51, 52, 53, 54, 55, 56, 58, 59, 60, 61, 62, 63, 64, 65, 66, 67, 68, 72, 73, 74, 75, 76, 81, 82, 91, 104, 105, 112, 117, 118, 132, 133, 134, 138, 139, 140, 141, 142, 143, 144, 149, 150, 159, 172, 173, 180, 185, 186, 200, 201, 202, 206, 207, 210, 211, 212, 213, 215, 216, 217, 2.18, 219, 220, 221, 227, 240, 241, 248, 253, 254, 255, 268, 269, 270, 274, 275, 278, 279, 280, 281, 283, 284, 285, 286, 287, 288, 289, 295, 308, 309, 316, 321, 322, 336, 337, 338, 342, 343.

The present composition is suitable for the prevention or management of autoimmune haemolytic anaemia. The commonest form of this disease is caused by IgG autoantibodies reactive against the Rh proteins. Conveniently, when the condition is AIHA the epitope is SEQ ID number 1, 2, 3, 5, 6, 8, 10, 11, 20, 21, 23, 25, 27, 33, 35, 37, 39, 41, 44, 46, 47, 50, 54, 56, 60, 62, 63, 65, 67, 68, 72, 73, 74, 76, 81, 82, 91, 104, 112, 117, 118, 133, 134, 138, 139, 140, 141, 142, 144, 149, 150, 159, 172, 180, 185, 186, 201, 202, 206, 207, 213, 215, 217, 218, 227, 240, 248, 253, 254, 269, 270, 274, 275, 281, 283, 285, 286, 295, 308, 316, 321, 322, 337, 338, 342, 343.

Conveniently the epitope or immunoreactive derivative is synthesised.

If the epitope sequences are artificially synthesised then microbial contamination is negligible.

Conveniently the epitope is disposed in a pharmaceutically acceptable vehicle.

Conveniently said vehicle is in an injectable, oral, rectal, topical or spray-uptake form.

Mammals may be tolerised to certain proteins or peptides by uptake of relatively small amounts of the specific protein or peptide through, for example, mucosal tissue, transdermal tissue or via the gut. Accordingly, proteins or fragments thereof for use in the present invention can be administered to a subject in need of tolerisation via, for example, mucosal tissue, and effectively tolerise the subject without causing an effector immune response.

In an injectable form the epitopes can be used deliberately to immunise the subject with an epitope, which can, for example, produce IL-10 or TGF-β1, which have immunosuppressive effects.

Conveniently the pharmaceutically acceptable vehicle is configured for transdermal or transmucosal administration or said vehicle is a formulation with an enteric coating for oral administration.

According to a further aspect of the present invention there is provided a method of treating or managing a condition caused by the alloimmunisaton or autoimmunity of a subject by Rh protein, the method comprising administering an immunologically effective epitope of a Rhesus protein to the subject.

Conveniently the condition is HDN or autoimmune haemolytic anaemia.

Conveniently the Rh protein is selected from RhD, RhcE, RhCe, RhCE.

Conveniently the Rh protein is RhD.

Preferably when the condition is haemolytic disease of the newborn, the epitope is selected from the group consisting of at least one of SEQ ID numbers 1, 2, 3, 4, 5, 6, 7, 8, 9, 10, 11, 12, 13, 14, 15, 16, 17, 18, 19, 20, 21, 22, 24, 25, 26, 27, 28, 29, 33, 34, 35, 36, 37, 40, 41, 43, 44, 45, 46, 47, 48, 49, 50, 51, 52, 53, 54, 55, 56, 58, 59, 60, 61, 62, 63, 64, 65, 66, 67, 68.

Preferably when the condition is autoimmune haemolytic anaemia, the epitope is selected from the group consisting of at least one of SEQ ID numbers 1, 2, 3, 5, 6, 8, 10, 11, 20, 21, 23, 25, 27, 33, 35, 37, 39, 41, 44, 46, 47, 50, 54, 56, 60, 62, 63, 62, 63, 65, 67, 68.

Conveniently the epitope is administered transdermally, transmucosally, or orally.

According to a yet further aspect of the present invention there is provided a method for determining effect of one or more epitopes from a rhesus protein on a human lymphocyte, in vitro, the method comprising:—
a) stimulating the lymphocyte with one or more epitope/peptide of a rhesus protein;
b) between 4 and 7 days later resuspending the cultures and transferring aliquots into plates prepared in the following manner;
c) coating each well in the plate with monoclonal anti-cytokine capture antibody;
d) washing the plate at least once with Hanks Buffered Salt Solution (HBSS);
e) blocking any non-specific binding using an appropriate solution;
f) incubating the plates with the lymphocyte culture for 12-36 hours at 30-40° C. in an atmosphere of substantially 5% CO₂ and substantially 95% air;
g) washing the plates at least once with Tween/PBS;
h) introducing an appropriate biotinylated monoclonal detection antibody to each well and incubating for 30-60 minutes at room temperature;
i) washing the plates at least once with Tween/PBS;
j) introducing ExtrAvidin-alkaline phosphatase conjugate and incubating for 15-45 mins;
k) washing the plates at least once with Tween/PBS;
l) developing the plates with 50-150 μl per well of p-nitrophenyl phosphate in 0.05M carbonate alkaline buffer pH9.6 added to each well;
m) reading the absorbance at 405 nm.

Traditionally, among other techniques, researchers have used a capture assay called ELISPOT to determine the size of the response from the number of cells that secrete cytokines. This assay produces a colour spot for each cytokine producing cell. A crude calculation based on the number of coloured spots is then used to estimate the amount of cytokines produced. The use of p-nitrophenyl phosphate in the present assay allows the amount of cytokine captured by the antibody in the well to be established on the basis of the colour change produced which can be measured by the more accurate method of spectrophotometry.

Accordingly, this method is very sensitive and therefore can identify that a particular RhD protein is capable of stimulating human T-cells to produce potentially immunosuppressive cytokines rather than to proliferate. This is important for the determination of the method of delivery of an epitope. An epitope, which leads to T-cell proliferation, may be given as a tolerogen through the nasal or mucosal route whereas an epitope, which leads to immunosuppresive cytokines, may be injected.

The invention will now be described, by way of illustration only, with reference to the following examples and the accompanying figures.

FIG. 1 shows the full amino acid sequence for RhD, RhcE, Rhce, RhCe or RhCE protein. (Reference: The Blood Group Antigen Facts Book P94, Editor; M E Reid & C Lomas-Francis, Academic Press London). Published sequences differ at position 218. The sequence we originally submitted was the more widely cited variant Ile218, which is now thought to be a sequencing error, with Met218 now generally accepted as the correct residue.

FIG. 2 shows the distribution of stimulatory peptides responses by HLA-DRB1*1501 homozygous matched alloimmunised donor and AIHA patient. Upper panel shows the stimulatory RhD peptides, from peptides 1 to 68 as per Table 1 in an alloimmunised donor. Lower panel shows the distribution of stimulatory RhD peptides, from peptides 1 to 68 as per Table 1, in an AIHA patient. X-RhD peptide stimulus. Y-Proliferative response (mean CPM×10⁻³+/−SD)

FIG. 3 shows the response pattern of the induction of proliferation and production of cytokines IFN-γ, TGF-β1 and IL-10 by T-cells after incubation with RhD peptides, from peptides 1 to 68 as per Table 1, in a RhD alloimmunised donor. No IL-4 was detected. X=RhD peptide stimulus, V=IL-10 secretion (pg/ml); W=TGF-β1 secretion (pg/ml); Y═IFN-γ secretion (pg/ml); Z=proliferative response (mean cpm×10-3+/−SD).

FIG. 4 shows the response pattern of the induction of proliferative and production of cytokines IFN-γ, TGF-β1 and IL-10 by T-cells after incubation with RhD peptides, from peptides 1 to 68 as per Table 1, in a RhD-positive autoimmune haemolytic anaemia patient. No IL-4 was detected. X=RhD peptide stimulus, V=IL-10 secretion (pg/ml); W=TGF-β1 secretion (pg/ml); Y═IFN-γ secretion (pg/ml); Z=proliferative response (mean cpm×10-3+/−SD). NT means not tested

FIG. 5 shows the response pattern of the induction of proliferative and production of cytokines IFN-γ, TGF-β and IL-10 by T-cells after incubation with Rhce peptides, from peptides 1 to 68 as per Table 2, in a RhD-negative autoimmune haemolytic anaemia patient. No IL-4 was detected. X=Rhce peptide stimulus, V=IL-10 secretion (pg/ml); W=TGF-β1 secretion (pg/ml); Y═IFN-γ secretion (pg/ml); z=proliferative response (mean cpm×10-3+/−SD).

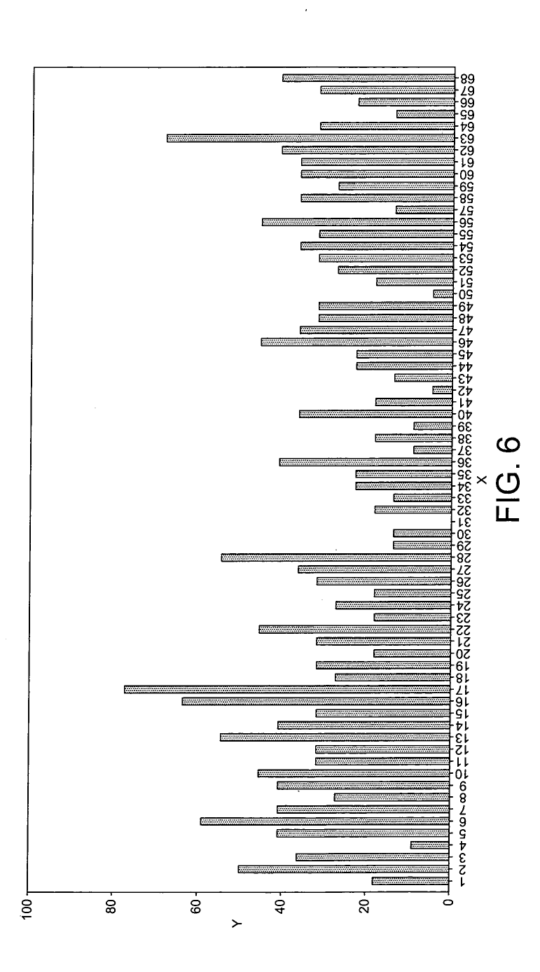

FIG. 6 shows the distribution of stimulatory RhD peptides in donors alloimmunised with RhD antigen from peptides 1 to 68 as per Table 1; X=RhD peptide added to culture; Y=percentage of subjects responding to specific RhD peptides.

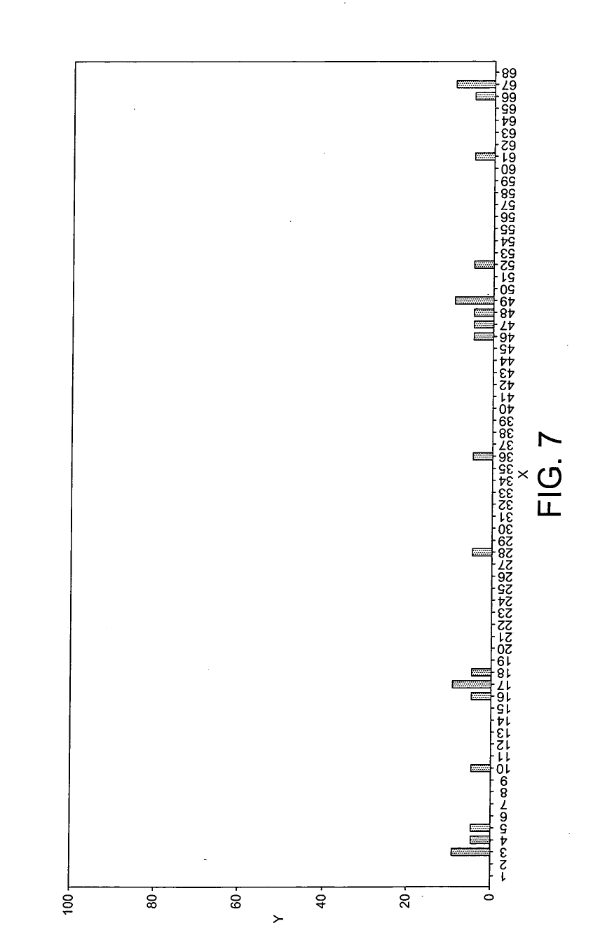

FIG. 7 shows the distribution of stimulatory RhD peptides in RhD-negative healthy control donors; from peptides 1 to 68 as per Table 1; X=RhD peptide added to culture; Y=percentage of subjects responding to specific RhD peptides.

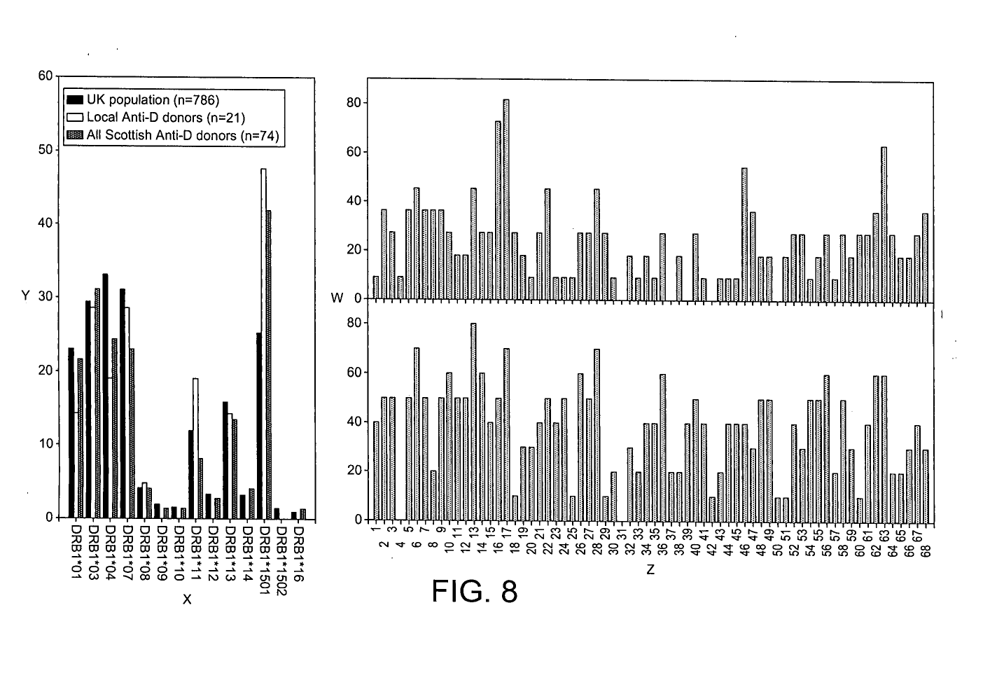

FIG. 8 shows the over-representation of the HLA-DRB1*1501 allele in RhD-negative donors selected for the ability to make anti-D antibodies in response to RhD alloimmunisation. Y=% of group carrying each allele; X=DRB1* allele tested. Also shows that Th-cells from anti-D positive alloimmune donors who carry HLA-DRB1*1501 proliferate in response to more RhD peptides than those with other HLA-DR types. Z=RhD peptide added to culture; W=percentage of subjects responding to specific RhD peptides.

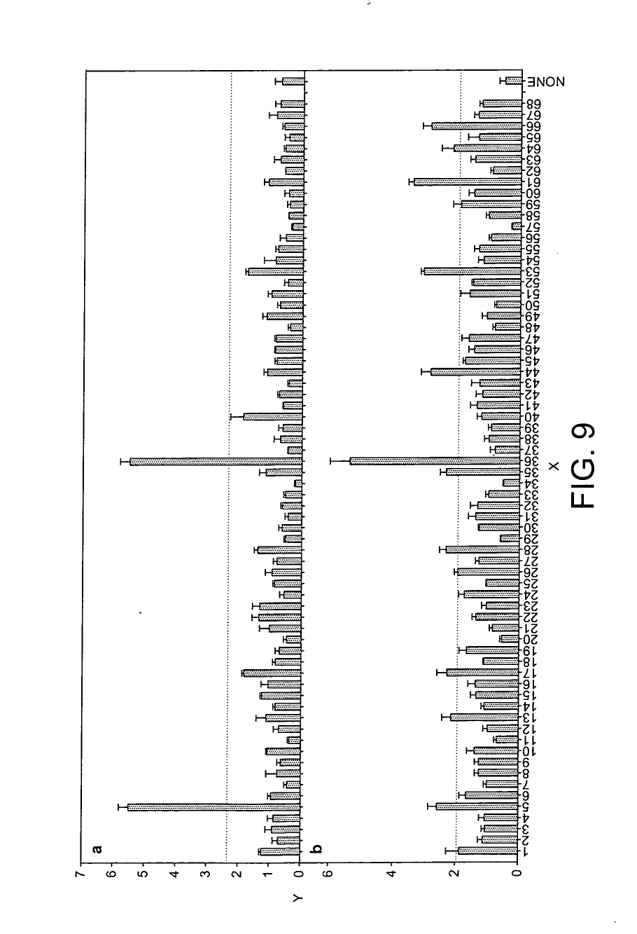

FIG. 9 shows similarity of responses in RhD alloimmunised donors (A and B) with the same DR type [DRB1*1501 homozygous]. X=RhD peptide added to culture. Y=proliferative response (mean cpm×10-3+/−SD).

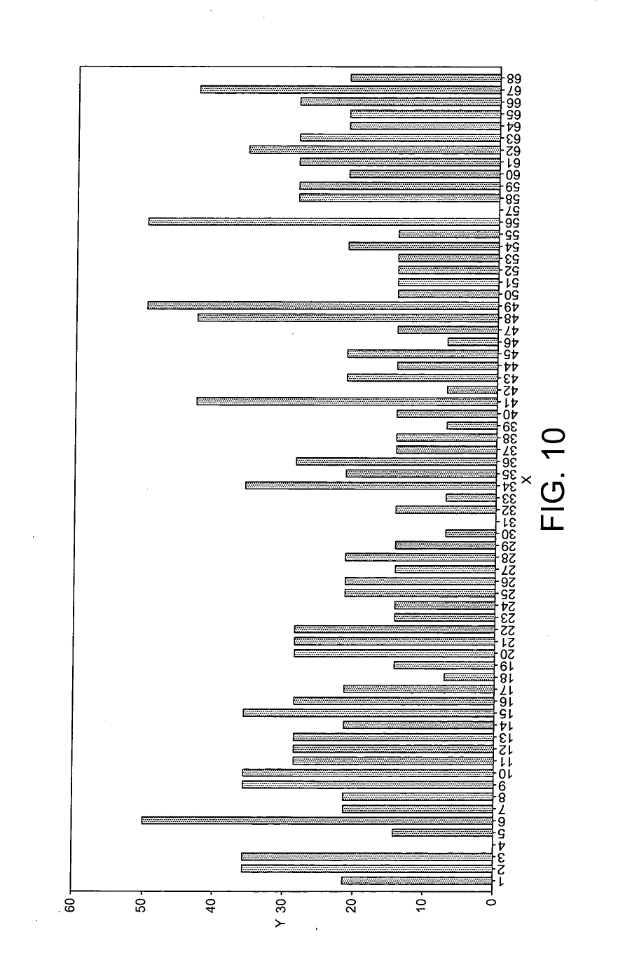

FIG. 10 shows the percentage of RhD alloimmunised donors (n=14) whose PBMC mounted significant IL-10 production in response to the RhD peptide panel, from peptides 1 to 68 as per Table 1. X=RhD peptide added to culture; Y=percentage of subjects responding to specific RhD peptides.

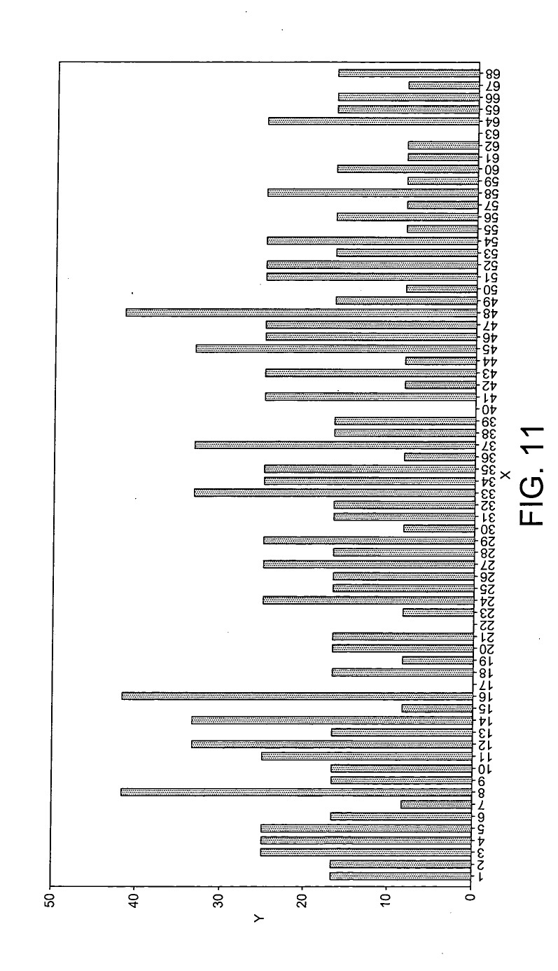

FIG. 11 shows the percentage of RhD alloimmunised donors (n=12) whose PBMC mounted significant TGF-β1 production in response to the RhD peptide panel, from peptides 1 to 68 as per Table 1. X=RhD peptide added to culture; Y=percentage of subjects responding to specific RhD peptides.

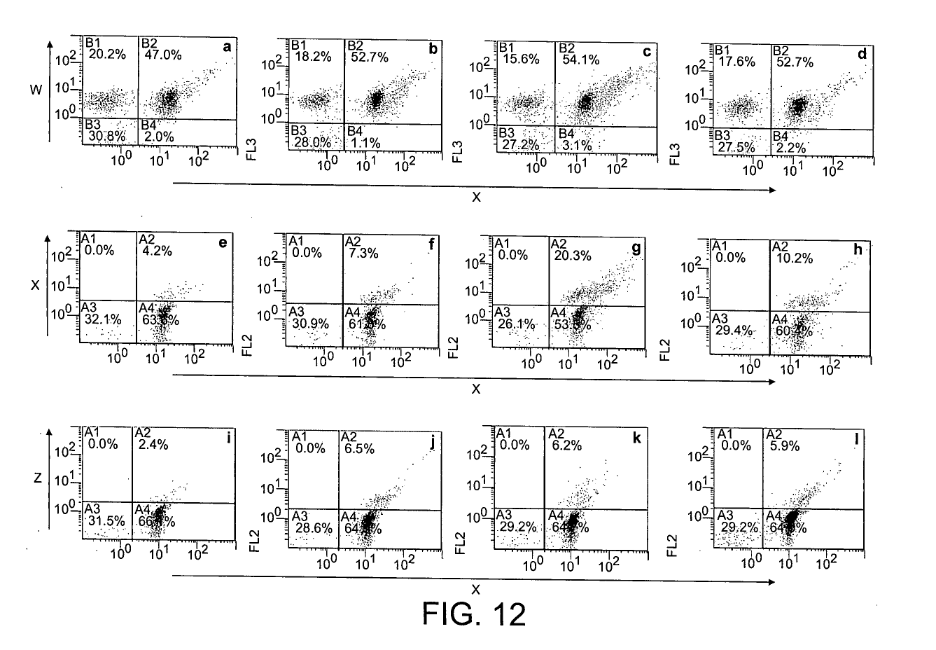

FIGS. 12A to 12L show the PBMC isolated from RhD alloimmunised donors producing IFN-γ and IL-10 in response to RhD peptides are CD3+ CD4+ T-cells. W═CD3; Y═IFN-γ; Z=IL-10; X═CD4; FIGS. 12A, 12E and 12I—unstimulated; FIGS. 12B, 12F and 12J—RhD; FIGS. 12C, 12G and 12K—RhD peptide 13, FIGS. 12D, 12H and 12L—RhD peptide 17.

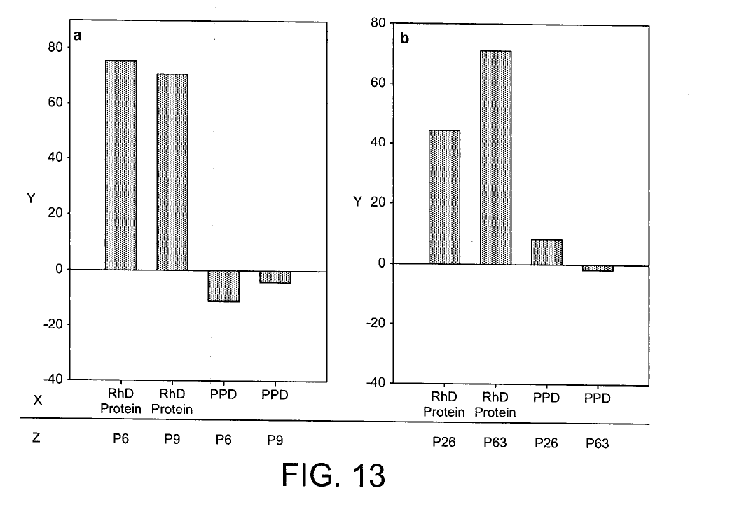

FIGS. 13A and 13B show the proliferative responses to the RhD protein, but not the PPD antigen, can be inhibited by the addition of IL-10 inducing RhD peptides in alloimmunised donors. FIG. 13A=Donor 4, FIG. 13B=Donor 6. X=stimulus; Z=IL-10 inducing peptide; Y=percentage inhibition of proliferative response.

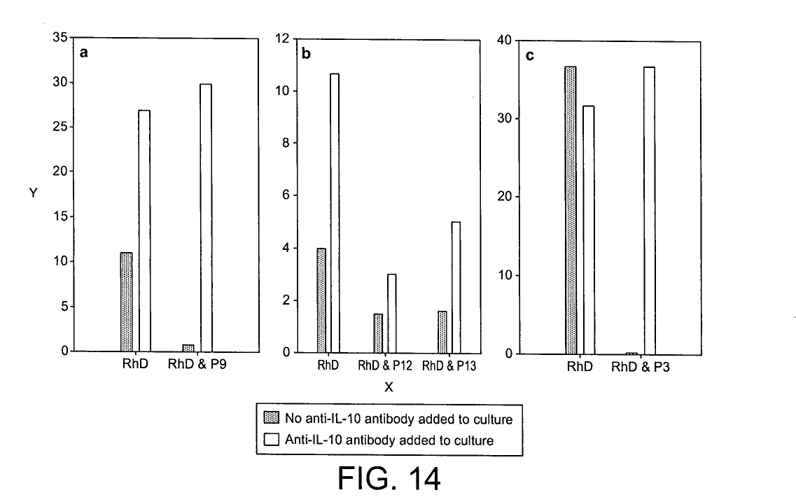

FIG. 14A to 14C show the reversal of the inhibitory effect of IL-10 inducing peptides by anti-IL-10 monoclonal antibody. FIG. 14A=donor 4; FIG. 14B=donor 10; FIG. 14C=donor 11; X=stimulus; Y=proliferation (stimulation index) shows the reversal of the inhibitory effect of IL-10 inducing RhD peptides by anti-IL-10 in an alloimmunised donor.

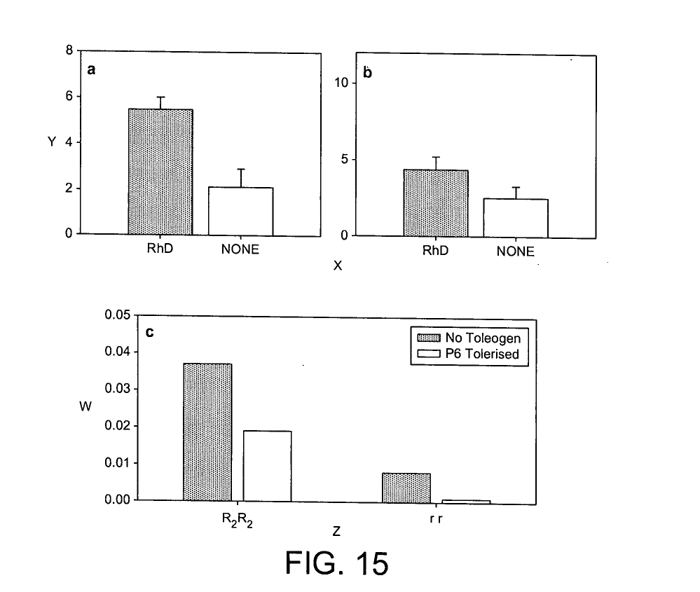

FIGS. 15A to 15C show tolerisation in vivo in HLA-DR15 transgenic mice to the RhD protein by IL-10 inducing peptide (p60) administered parenterally (subcutaneous). FIG. 15A shows T-cell response to the RhD compared to unstimulated cultures by T cells from RhD immunised mice. FIG. 15B shows reduced T-cell response to RhD protein after prior tolerisation with RhD peptide p60. FIG. 15C shows reduction of anti-human RBC antibody with prior tolerisation with p60 in R2R2 red blood cells but no change in rr red blood cells. X=stimulus; Y=proliferation (mean CPM×10⁻³+/−SD); W═OD₄₀₅₋₄₉₂; Z=RBC phenotype

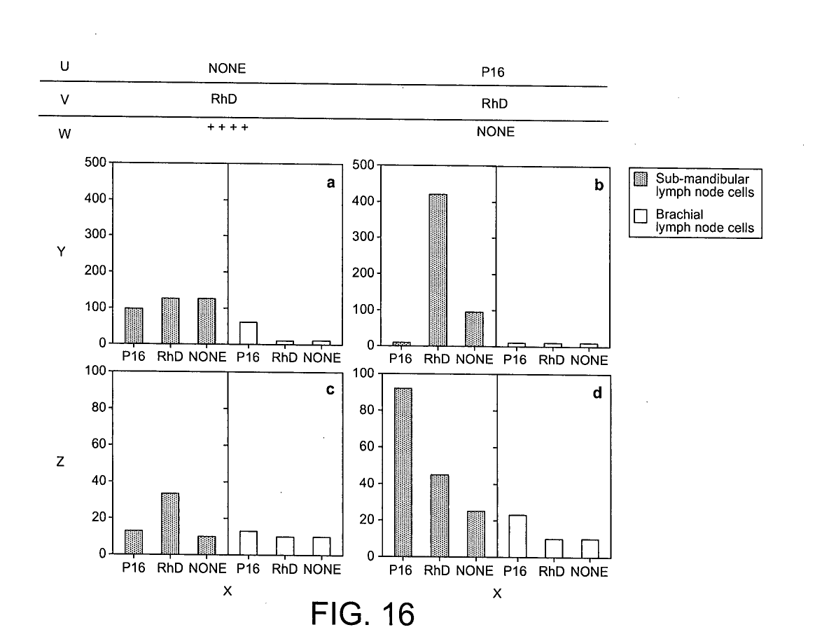

FIG. 16A to 16D show that tolerance induced in vivo in HLA-DR15 transgenic mice by a dominant RhD peptide (p16) is associated with an increase in both TGF-β1 and IL-10 production by cells isolated from the draining lymph node. FIGS. 16A and 16C show the production of cytokine by lymph node cells isolated from mice that have been immunised with RhD protein. FIGS. 16B and 16D show the production of cytokines by lymph node cells isolated from mice that have been tolerised with RhD peptide 16 prior to RhD immunisation. U=nasal administration of peptide; V=immunisation; W=serum anti human red blood cell IgG; Y=production of TGF-β1 (pg/ml); Z=production of IL-10 (pg/ml); X=stimulus.

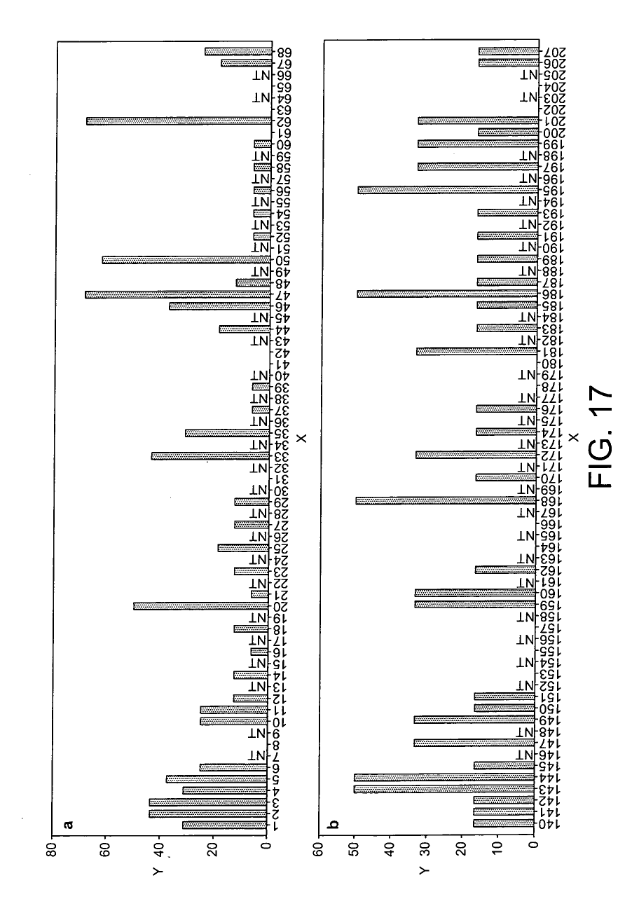

FIGS. 17A and 17B show the distribution of stimulatory Rh peptides in autoimmune haemolytic anaemia patients. FIG. 17A shows the distribution of stimulatory RhD peptides, from peptides 1 to 68 as per Table 1, in RhD-positive AIHA patients (n=16). FIG. 17B shows the distribution of stimulatory RhCE peptides, from peptides 140 to 207 as per Table 3, in AIHA patients (n=8). X=Rh peptide added to culture; Y=percentage of subjects responding to specific RhD peptides; NT=not tested.

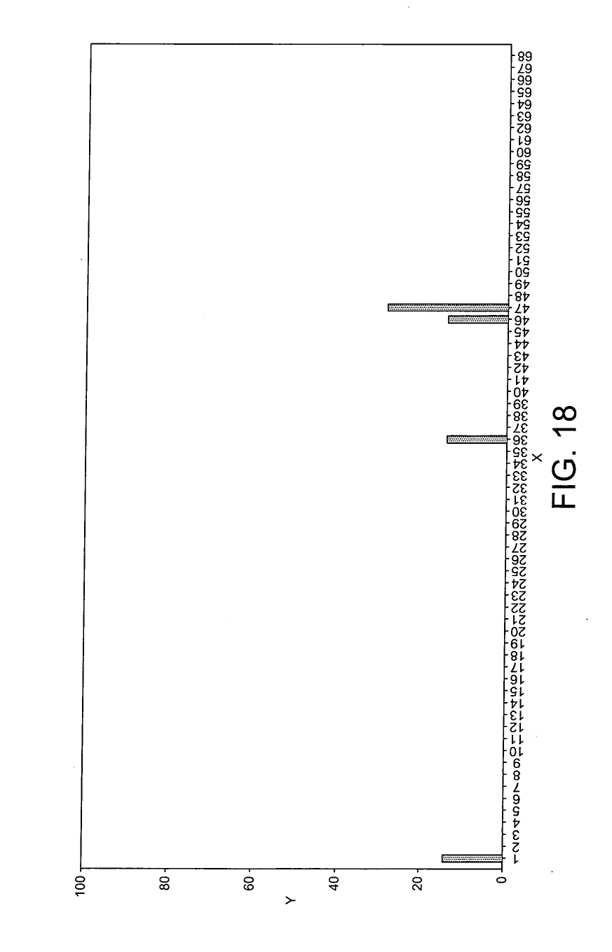

FIG. 18 shows the distribution of stimulatory RhD peptides in RhD positive healthy control donors; from peptides 1 to 68 as per Table 1; X=RhD peptide added to culture; Y=percentage of subjects responding to specific RhD peptides.—

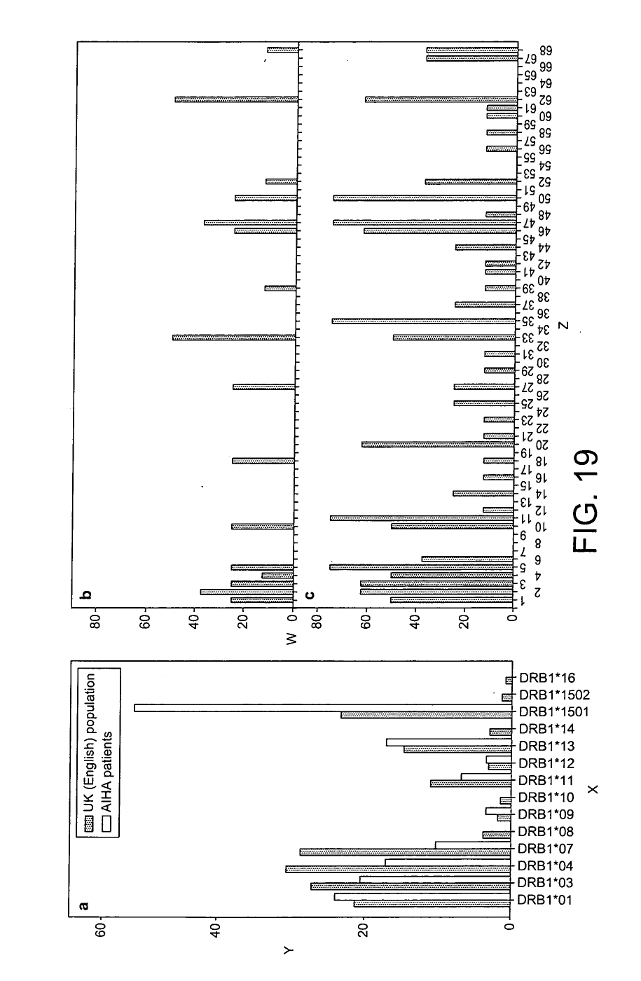

FIG. 19A to 19C show the over-representation of the HLA-DRB1*1501 allele in patients with autoimmune haemolytic anaemia. Y=% of group carrying each allele; X=DRB1* allele tested. Also shows that Th-cells from the patients who carry HLA-DRB1*1501(FIG. 19C) proliferate in response to more RhD peptides than those with other HLA-DR types (FIG. 19B). Z=RhD peptide added to culture; W=percentage of subjects responding to specific RhD peptides. NT means not tested.

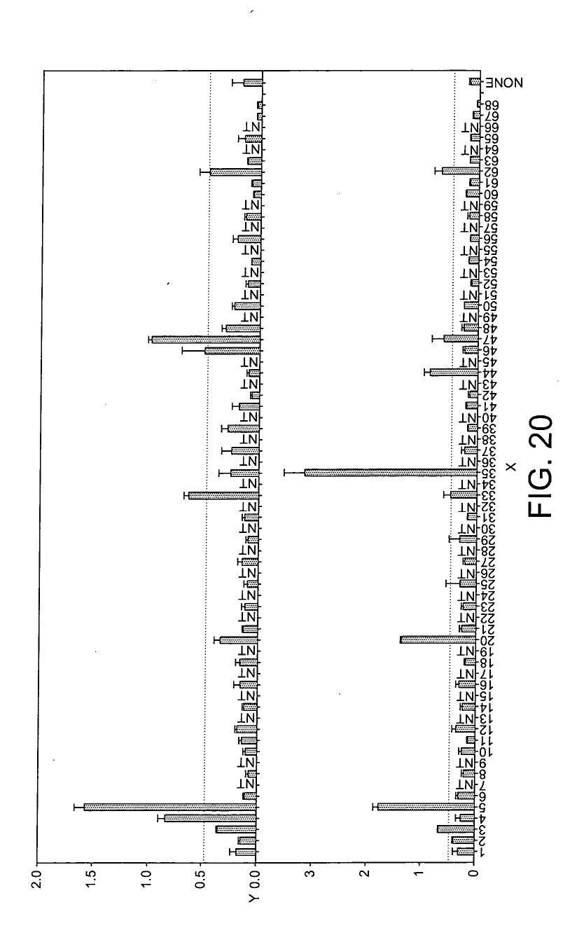

FIG. 20 shows that some of the variation in Th cell epitopes between individuals in the alloimmunised donor group or the AIHA patient group can be attributed to differences in HLA type. Thus, when two AIHA patients were matched for HLA type (HLA-DRB1*1501, HLA-DRB1*11) the profile of responses was found to be positively associated (Rₛ=0.515, p<0.001). X=RhD peptide added to culture; Y=proliferative response to peptide by PBMC. NT=not tested.

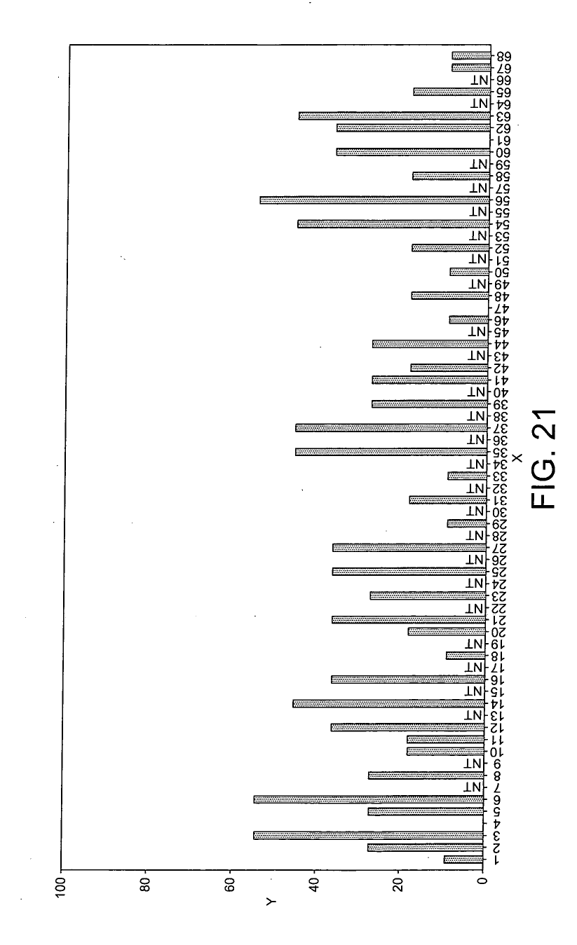

FIG. 21 shows the percentage of autoimmune haemolytic anaemia patients (n=11) whose PBMC mounted significant IL-10 production in response to the RhD peptide panel, from peptides 1 to 68 as per Table 1. X=RhD peptide added to culture; Y=percentage of subjects responding to specific RhD peptides. NT=not tested.

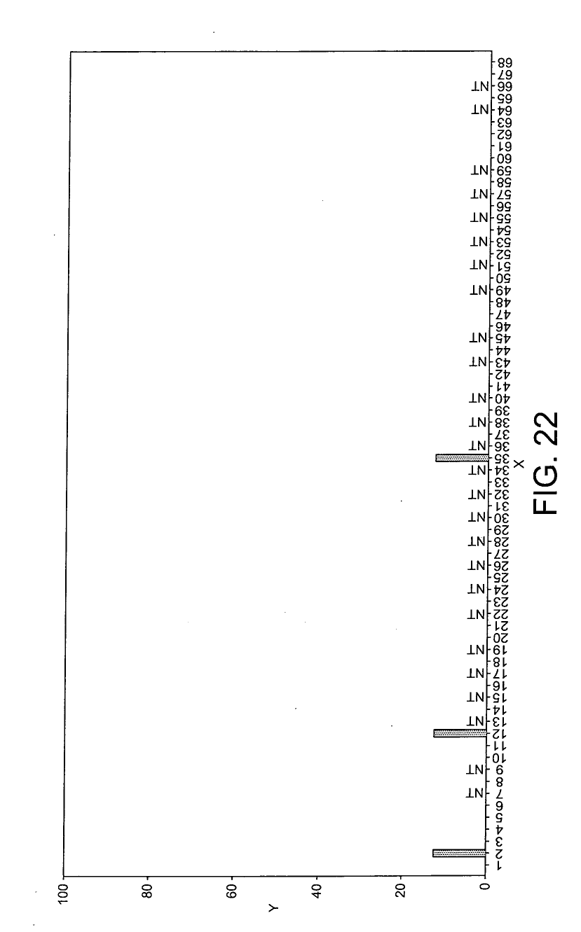

FIG. 22 shows the percentage of autoimmune haemolytic anaemia patients (n=8) whose PBMC mounted significant TGF-β1 production in response to the RhD peptide panel, from peptides 1 to 68 as per Table 1. X=RhD peptide added to culture; Y=percentage of subjects responding to specific RhD peptides.

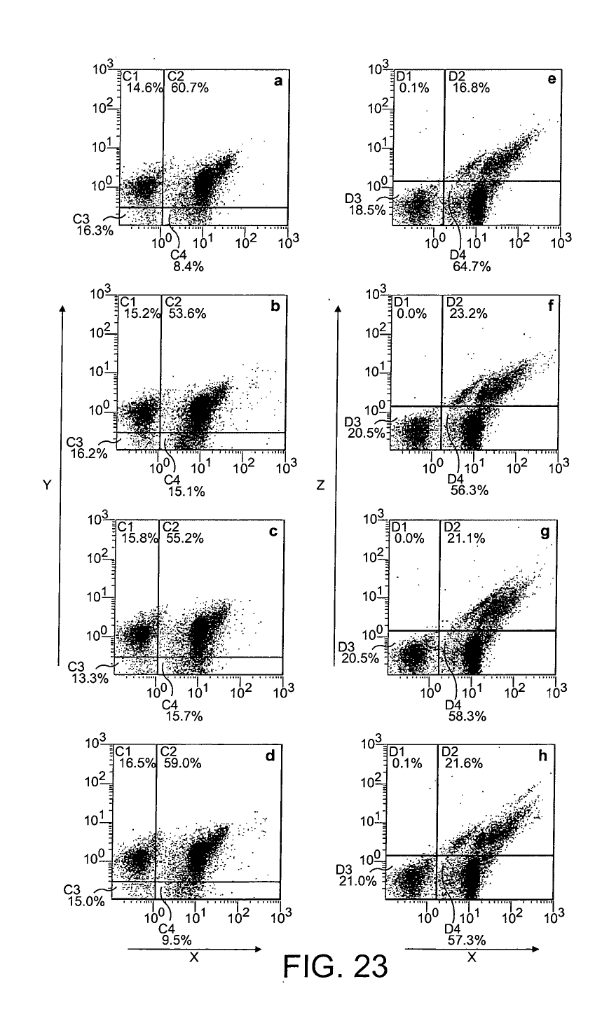

FIG. 23A to 23H show that, in an AIHA patient, the stimulation with RhD protein or RhD peptides is associated with the expansion of a CD3+ CD4+population that secrete IFN-γ. FIGS. 23A and 23E show unstimulated cells; FIGS. 23B and 23F show RhD stimulated cells, FIGS. 23C and 23G show RhD peptide p35 stimulated cells. FIGS. 23D and 23H show RhD peptide p60. X═CD4; Y═CD3; Z=intracellular IFN-γ

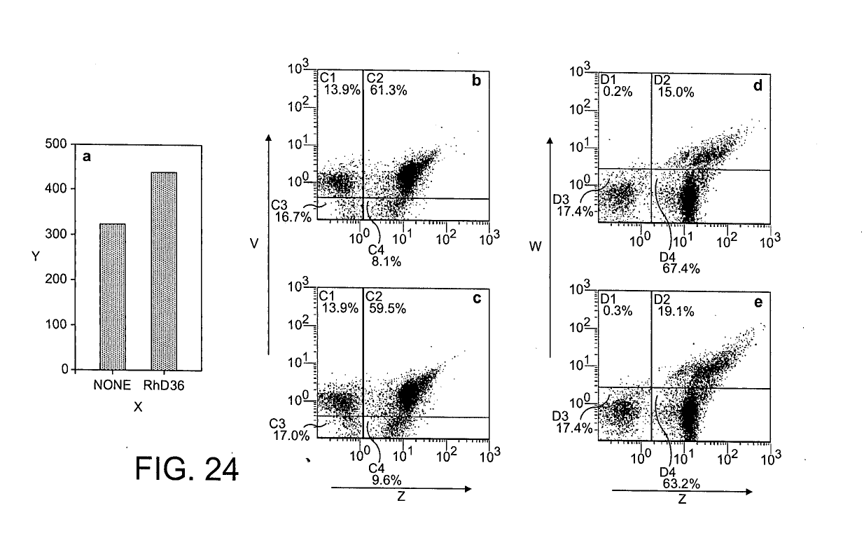

FIG. 24A to 24E shows that increased IL-10 production following stimulation of PBMC from AIHA26 with the RhD peptide p36 is associated with T-cells. FIG. 24A shows that increased IL-10 can be detected in P36 stimulated cultures. FIGS. 24B and 24C show the populations of IL-10 secreting T cells in unstimulated cultures. FIGS. 24D and 24E show the population of IL-10 secreting T cells in p36 stimulated cultures. V=CD3; W=intracellular IL-10; X=Stimulus; Y═IL-10 production (pg/ml); Z=CD4

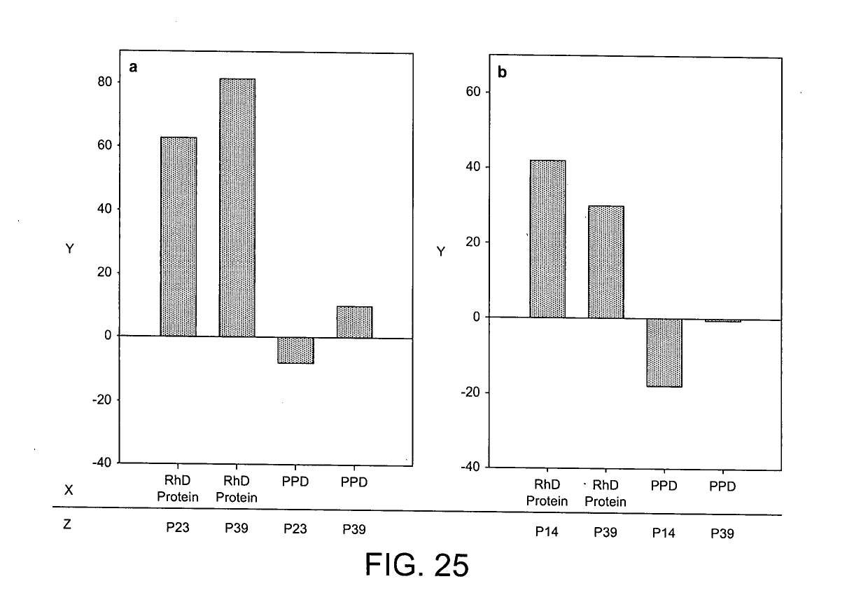

FIGS. 25A and 25B show the proliferative responses to the RhD protein, but not the PPD antigen, can be inhibited by the addition of IL-10 inducing RhD peptides in AIHA patients. FIG. 25A=AIHA patient 6, FIG. 25B=AIHA patient 26. X=stimulus; Z=IL-10 inducing peptide; Y=percentage inhibition of proliferative response.

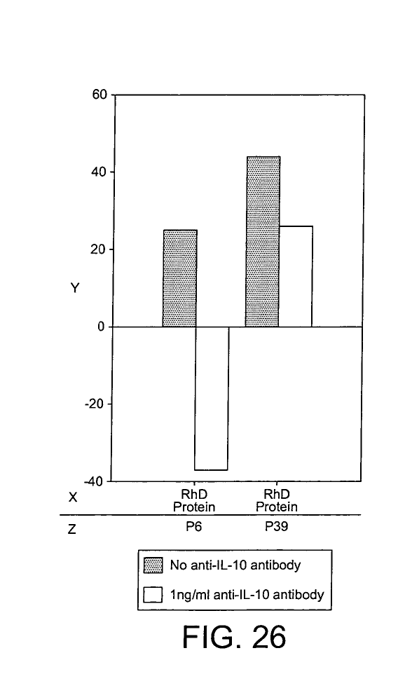

FIG. 26 shows the reversal of the inhibitory effect of IL-10 inducing RhD peptides by anti-IL-10 in AIHA patient 26. X=stimulus, Z=IL-10 inducing peptide; Y=percentage inhibition of proliferative response.

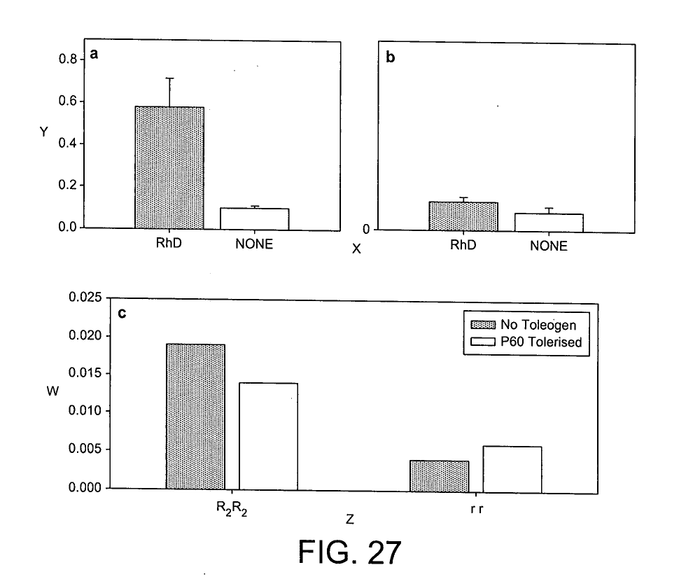

FIGS. 27A to 27C show tolerisation in vivo in HLA-DR15 transgenic mice to the RhD protein by a dominant proliferative RhD peptide (p6) administered by the nasal mucosae. FIG. 27A shows T-cell response to the RhD compared to unstimulated cultures by T cells from RhD immunised mice. FIG. 27B shows reduced T-cell response to RhD protein after prior tolerisation with RhD peptide p6. FIG. 27C shows reduction of anti-human RBC antibody with prior tolerisation with p6 in R₂R₂ red blood cells and rr red blood cells. X=stimulus; Y=proliferation (mean CPM×10⁻³+/−SD); W═OD405-492; Z=RBC phenotype

### EXAMPLE 1

### Relating to Therapy for Haemolytic Disease of the Newborn

### EXAMPLE 1.1

Five complete panels of 68 15-mer peptides, with 5 or 10 amino acid overlaps, were synthesized (Multiple Peptide Service, Cambridge Research Biochemicals, Cheshire, UK and Dept. Of Biochemistry, University of Bristol, UK), corresponding to the sequences of the 30 kD Rh proteins associated with expression of the RhD or RhcE/ce/Ce/CE blood group antigens respectively (Tables 1 to 5). An additional three peptide sequences is added to the panel of RhD peptides to account for the Ile to Met substitution described earlier (numbered from 69 to 71). The amino acid sequences for each of these proteins were deduced from cDNA analyses (FIG. 1). In order to ensure purity, each panel was synthesized by fluorenylmethoxycarbonyl chemistry on resin using a base-labile linker, rather than by conventional pin technology, and randomly selected peptides were screened for purity by HPLC and amino acid analysis. The peptides were used to stimulate cultures at 20 μg/ml, although it should be noted that the responses of the cultures had previously been shown to be similar in magnitude and kinetics at peptide concentrations between 5-20 μg/ml.

The control antigens Mycobacterium tuberculosis purified protein derivative (PPD) (Statens Seruminstut, Denmark) and keyhole limpet haemocyanin (KLH) (Calbiochem-Behring, La Jolla, Calif., USA) were dialysed extensively against phosphate buffered saline pH7.4 (PBS) and filter sterilized before addition to cultures at 50 μg/ml, PPD, but not KLH, readily provokes recall T-cell responses in vitro, since most UK citizens have been immunised with BCG. Concanavalin A (Con A) was obtained from Sigma, Poole, Dorset, UK, and used to stimulate cultures at 10 μg/ml.

Whole RhD protein was isolated from homozygous D-positive blood on magnetic beads coated in anti-D antibody. The volume of packed red cells (PRBC) required for a given amount of RhD protein is given by

$$pRBC = \frac{( weight RhD required [ g ] \text{/} weight of RhD on a RBC [ g ] )}{No . erythrocytes in 1 m packed RBCs}$$

Homozygous RhD-positive blood was washed extensively with PBS before 400 μl packed red blood cells were incubated with 500 μl of one of two monoclonal anti-RhD antibodies, Therad 19 (epD4) or Therad 27 (epD1), and gently rotated for one hour at 37° C. The cells were centrifuged at 2000 rpm for 10 minutes at room temperature and the unbound anti-D antibody in the supernatant collected and stored.

Isolation of Purified RhD Protein

Erythrocytes were re-suspended in HBSS (Gibco BRL) and then centrifuged at 2000 rpm for 10 minutes at room temperature. The supernatant was removed and the washing step was repeated twice. After the final wash erythrocyte ghosts were prepared by hypotonic lysis in erythrocyte lysis buffer and incubated for 15 minutes at room temperature. The cells were centrifuged at 20,000 rpm at 4° C. for 30 minutes. In the absence of complete erythrocyte lysis this step was repeated. Once the suspension was clear of haemoglobin the pellet was dissolved in 2% Triton X100 (Sigma) before centrifuging again at 20,000 rpm, 4° C. for a further 30 minutes to remove insoluble debris. 1 ml of anti-human IgG coated magnetic beads (Metachem Diagnostics) were washed six times in HBSS and re-suspended in 10 ml HBSS. The red cell lysate was added to 1 ml anti-human IgG coated beads and was gently rotated overnight at room temperature. The beads coated with RhD were then washed six times in HBSS and re-suspended to 2 mg/ml RhD protein coating the magnetic beads in HBSS and stored at 4° C.

Antibodies

FITC- or phycoerythrin-conjugated mabs against human CD3, CD19, CD45 or CD14 were obtained from Dako UK Ltd. Blocking mAbs specific for HLA-DP, -DQ, or -DR supplied by Becton Dickinson (Oxford, UK) were dialysed thoroughly against PBS before addition to cultures at the previously determined optimum concentration of 2.5 μg/ml.

Isolation of Peripheral Blood Mononuclear Cells and T-Cells

Peripheral blood mononuclear cells (PBMC) from donors or patients were separated from fresh blood samples using Ficoll-Hypaque. The donors and patients had become alloimmunised with RhD-positive blood either through pregnancy, a blood transfusion or through immunization with the relevant blood.

The viability of PBMC was greater than 90% in all experiments, as judged by trypan blue exclusion. T-cells were isolated from PBMC by passage through glass bead affinity columns coated with human IgG/sheep anti-human IgG immune complexes. Flow cytometry (Becton Dickinson FACScan) demonstrated that typical preparations contained more than 95% T-cells.

Cell Proliferation Assays

PBMC were cultured in 100 μl volumes in microtitre plates at a concentration of 1.25×10⁶ cells/ml in an Alpha Modification of Eagle's Medium (ICN Flow, Bucks UK) supplemented with 5% autologous serum, 4 mM L-Glutamine (Gibco, Paisley, UK), 100 U/ml sodium benzylpenicillin G (Sigma), 100 μg/ml streptomycin sulphate (Sigma), 5×10⁻⁵M 2-mercaptoethanol (Sigma) and 20 mM HEPES pH7,2 (Sigma). All plates were incubated at 37° C. in a humidified atmosphere of 5% CO₂/95% air. The cell proliferation in cultures was estimated from the incorporation of ³H-Thymidine in triplicate wells 5 days after stimulation with antigen as described previously. Proliferation results are presented either as the mean CPM+/−SD of the triplicate samples, or as a stimulation index (SI), expressing the ratio of mean CPM in stimulated versus unstimulated control cultures. An S1>3 with CPM>1000 is interpreted as representing a positive response.

Activation Assay

The aforementioned experiments were designed to minimise the response by quiescent or naive T-cells that can recognise RhD protein, but which are not activated by immunisation. To validate the experiments, the T-cells proliferated in the aforementioned experiment were tested using a modification of the method set out in European Journal of Immunology (1994) 24: 1578-1582 to identify if they had been activated in vivo. In this connection, the T-cells were screened to ascertain if they were from the subset bearing CD45RO, which is a marker of previous activation or “memory”, rather than from the subset bearing CD45RA, which is the marker of quiescent, or “naive” T-cells.

Accordingly we have shown that helper T-cells from all donors deliberately immunised against RhD can be stimulated in vitro by RhD peptides.

Further there is a variation between alloimmune donors in the T-cell response profile to the RhD peptides. Despite these variations, RhD peptides Nos. 2, 6, 10, 13, 16, 17, 22, 28, 46, 56 and 63 are most commonly targeted and a proliferative response was elicited by peptide 17 in 70% of donors (see FIG. 6). By comparison, PBMC from RhD-negative control donors rarely proliferate in response to RhD peptides (see FIG. 7).

It is predicted that alloreactive T-cell epitopes on the RhD protein would comprise sequences that are foreign to RhD-negative individuals, and would thus not be carried on the related RhCc/Ee protein that is expressed on the erythrocytes of such individuals. With the exception of peptide 46, all of the fragments identified are sequences that fulfil this prediction. It is therefore considered that such peptides, or derived sequences, could be used to stimulate either T-cell response or tolerance in vivo as desired, depending on the route of administration and/or the dose and formulation of the preparation.

The T-cells, which proliferated were in fact drawn from those that have been previously activated. This is important because it is these cells that will drive anti-D antibody production in RhD-negative donors immunised with RhD.

It follows that the characterisation of the putative helper T-cell epitopes we have identified results in the development of safe immunogens for anti-immunoglobulin donors and allows the evaluation of peptide immunotherapy as a novel approach to the prevention of haemolytic disease inter alia in neonates.

### EXAMPLE 1.2

The HLA class II tissue type of the donors tested in Example 1.1 was ascertained by standard SSP-PCR methods. This was carried out because the molecules that determine tissue type select and bind antigenic peptide fragments for display to T-cells, therefore they are important in this investigation.

The techniques described in Barker et al (1997)Blood 90:2701-2715 were used to determine that the HLA-DR loci was more important than either the HLA-DP or HLA-DQ loci in the presentation of RhD peptide fragments that stimulate T-cells in vitro. A significant proportion of RhD-negative donors selected for responsiveness to RhD carry the HLA-DRB1*15 gene with 47.62% of alloimmunised donors carrying this HLA type (FIG. 8). It is likely therefore, that HLA-DRB1*15 preferentially enhances the likelihood of responses to epitopes from the RhD protein.

The group of alloimmunised donors tested in Example 1.1 was divided into two groups; those that express HLA-DRB1*1501 and those that do not; and the distribution of stimulatory epitopes was calculated. RhD peptides are more likely to induce proliferative responses by PBMC from HLA-DR15+ allo-immunised donors compared to donors that do not express HLA-DR15. (see FIG. 8 upper and lower panels). Thus carrying this tissue type is associated with an increased risk of producing anti-D antibodies after exposure to RhD-positive erythrocytes.

The broad spectrum of stimulatory epitopes, from the RhD protein, between alloimmunised individuals could reflect the different HLA class II molecules expressed by antigen presenting cells. Two donors were matched for HLA-DR and HLA-DQ type; specifically HLA-DRB1*1501 and HLA-DQB1*06; and the response profile of stimulatory RhD peptides compared (see FIG. 9). It was found that there is a strong positive correlation between these individuals (Rs=0.503, p<0.001).

A statistical analysis of all the data shows that the effect of HLA-DR type on the identity of the peptides recognised is relatively weak. In other words, many of the RhD peptides stimulate T-cells regardless of tissue type. For example, at least one of the four RhD peptides that are most likely to induce proliferation responses will induce a response by T-cells from all alloimmune donors tested.

These analyses demonstrate that the selection of RhD peptide fragments for immunisation/tolerisation regimes may not be dependent on prior tissue typing of recipients, an important practical consideration for the clinical application of this approach.

### EXAMPLE 1.3

The following Example identified RhD peptides that induce regulatory responses to the RhD protein in alloimmunised RhD negative individuals.

Cultured T-cells are stimulated with each of the epitopes given in Table 1 and after 5 days the responding cells were transferred to flat-bottomed microtitre plates (96-well Nunc-Immuno Maxisorp) coated with 501 per well of monoclonal anti-cytokine capture antibody diluted in 0.05M alkaline carbonate coating buffer pH 9.6. Unbound capture antibody was removed by two washes with HBSS and non-specific binding potential blocked by incubation with 200 μl per well of phosphate buffered saline, pH 7.4 (PBS containing 3% BSA).

Five days after stimulation, lymphocyte cultures were mixed to resuspend the cells and duplicate 100 μl aliquots were transferred into wells coated with the respective capture antibody specific for IFN-γ, IL-10 or TGF-β. The plates coated with capture antibodies and layered by lymphocytes were then incubated for a further 24 hours at 37° C. in a humidified atmosphere of 5% CO₂ and 95% air. After this incubation the PBMC were removed by four washes with 0.2% Tween/PBS. One hundred microlitre aliquots of the appropriate biotinylated monoclonal detection antibody in 0.2% BSA/PBS were then added to the wells and incubated at room temperature for 45 minutes.

After six washes with 0.5% Tween/PBS, 100 μl of 1:100,000 ExtrAvidin-alkaline phosphatase conjugate (Sigma) was then added to each of the wells and incubated at room temperature for 30 minutes. The ExtrAvidin conjugate was removed by eight washes with 0.2% Tween/PBS, and the plates developed using 100 μl per well of p-nitrophenyl phosphate (Sigma) 1.0 mg/ml in 0.05M carbonate alkaline buffer pH9.6. The absorbance at 405 nm was then measured using a Multiscan plate reader (Labsystems Basingstoke UK).

Cytokine secretion was measured by interpolation from a standard curve generated by incubating duplicate wells with doubling dilutions of recombinant human IFN-γ or IL-10 or TGF-β (Pharmingen). The standard curves were modelled by a smoothed cubic spline function applied to the absorbance reading and the cytokine concentrations after a quasilogarithmic transformation, where:
quasilogₑ(z)=logₑ[z+√[z²+1]).

This method is very sensitive and therefore can identify that a particular RhD peptide is capable of stimulating human T-cells to produce potentially immunosuppressive cytokines rather than to proliferate.

From FIG. 3 it can be seen that the proliferative response to RhD peptides by PBMC from an alloimmunised donor is often associated with the production of IFN-γ. Although relatively small amounts of IL-4 are produced by PBMC from alloimmunised donors proliferation is often associated with a significant increase in IL-4 production. The production of both these cytokines shows that the proliferative response has elements of both the Th1 and Th2 bias although relatively more IFN-γ is produced.

Using celELISA technique, the 68 RhD peptide panel was screened for the ability to induce IL-10 and TGF-β production for PBMC from a total of 19 samples from 12 different alloimmune donors. PMBC from 10 out of the 12 RhD-alloimmunised donors could produce IL-10 to at least one of the RhD peptides on at least one occasion, with a total of 194 significant IL-10 responses to the RhD peptides between all the donors. The RhD peptides that induced significant TGF-β or IL-10 and proliferative responses from PBMC in all the samples screened are summarised in FIGS. 10 and 11.

From FIG. 10 it can be seen that RhD peptides 6, 49 and 56 commonly induce the production of IL-10 by PBMC from alloimmunised donors. Importantly, some IL-10 inducing peptides do not induce either proliferation or the production of IFN-γ or IL-4 (for example, in FIG. 3, RhD peptide 6 and 49 induce IL-10 production in the absence of a proliferative response or the production of IFN-γ or IL-4). An analysis of the proportion of RhD-alloimmunised donors with PBMC that mounted significant responses to each of the 68 RhD peptides revealed that the following peptides stimulated PBMC from ≧20% of the donors to produce significant IL-10 (FIG. 10):
1, 2, 3, 6, 7, 8, 9, 10, 11, 12, 13, 14, 15, 16, 17, 20, 21, 22, 25, 26, 28, 34, 35, 36, 41, 43, 45, 48, 49, 54, 56, 58, 59, 60, 61, 62, 63, 64, 65, 66, 67, 68.
and more than 40% of donors produced significant IL-10 in response to peptides:
6, 41, 48, 49, 56, 67

From FIG. 11 it can be seen that RhD peptides 8, 16 and 48 commonly induce the production of TGF-β1. Importantly, some TGF-β1 inducing peptides do not induce either proliferation or the production of IFN-γ or IL-4 (for example, in FIG. 3, RhD peptide 48 induces TGF-β1 production in the absence of a proliferative response or the production of IFN-γ or IL-4). An analysis of the proportion of RhD-alloimmunised donors with PBMC that mounted significant responses to each of the 68 RhD peptides revealed that the following peptides stimulated PBMC from ≧20% of the donors to produce significant TGF-β1 (FIG. 11):
3, 4, 5, 8, 11, 12, 14, 16, 24, 27, 29, 33, 34, 35, 37, 41, 43, 45, 46, 47, 48, 51, 52, 54, 58 and 64
and more than 40% of donors produced significant TGF-β in response to peptides:
8, 16 and 48

Therefore, it is clear that the RhD peptides can induce the production of significant levels of both IL-10 and TGF-β from PBMC isolated from RhD-alloimmunised donors.

Some of the peptides from the RhD peptide panel contain sequences corresponding to the RhcE, Rhce, RhCe and RhCE proteins. Analysis shows that the regulatory T-cell responses are not focused on the peptides containing shared amino acid residues common to the RhD and, RhcE, Rhce, RhCe or RhCE protein or peptides specific for the RhD protein alone, in either RhD-negative or RhD-positive control donors (Chi square analysis, data not shown).

The PBMC of RhD-negative control donors (who have not been exposed to RhD-positive cells) rarely mounted proliferative responses to RhD (FIG. 7). However, the RhD peptides could induce regulatory cytokine responses, but when compared to the alloimmunised donors, there were significantly fewer IL-10 or TGF-β responses to the RhD peptides (Chi-squared analysis).

RhD peptides that induce IL-10 have been shown to inhibit T-cell proliferation in response to the entire RhD protein in vitro. Accordingly, prior administration of RhD peptides that elicit T-cell IL-10 production can be used to prevent RhD-negative individuals from responding to RhD in vitro. This novel approach to manipulating the immune system has other applications, including treatment of warm-type autoimmune haemolytic anaemia, in which the Rh proteins are important targets. The identification of peptides with similar properties derived from other antigens could also lead to therapy for a wide range of autoimmune diseases where the antigens/proteins are identified.

### EXAMPLE 1.4

The secretion of cytokines identified in Example 1.3 by T-helper cells is confirmed by flow cytometery analysis. Simultaneous measurement of surface molecules CD3 and CD4 and intracellular cytokines IFN-γ or IL-10 was carried out using three colour flow cytometry analysis in PBMC, isolated from RhD-alloimmunised donors, cultured with either no antigen or RhD peptide for five days after stimulation. All analysis was carried out using Expo v2 analysis software (applied Cytometry Systems). The CD3+ T-cells were gated open and the numbers of IFN-γ+ and CD4+ cells were assessed. In all donors tested there was a significant increase in the proportion of CD4+ IFN-γ+CD3+ T-cells to at least one of the RhD peptides tested. In the representative example shown in FIGS. 12A to 12L it can be seen that the proportion of CD4+ IFN-γ+cells was 4.2% in unstimulated cultures (FIG. 12E). In addition, there was a significant increase in the CD4+ IFN-γ T-cells (20.3% FIG. 12G) in culture stimulated with RhD peptide 17 and peptide 28 (10.2% FIG. 12H). RhD peptide 17 had previously been shown to be an immunodominant peptide that induces significant PBMC proliferation. The IL-10 and CD4+ expression was also assessed on CD3+ gated population of T-cells. In PBMC cultures isolated from the alloimmune donor shown, there was an increase in the proportion of CD4+ IL-10+T-cells from 2.4% in unstimulated PBMC cultures (FIG. 12I) to 6.5% in culture stimulated with RhD peptide 13 (FIG. 12J) to 6.2% in cultures stimulated with RhD peptide 17 (FIG. 12K) and 5.9% with RhD peptide 28 (FIG. 12L). This indicates that the PBMC producing IL-10 is CD3+ CD4+ Th-cells.

### EXAMPLE 1.5

Peptides that had previously elicited IL-10 but not proliferation were identified to see if they were capable of suppressing the proliferative response elicited to the whole RhD protein by the Th-cells.

Whole RhD protein was isolated as set out in Example 1.1.

IL-10 inducing peptides, which were identified in Example 1.3, were placed in culture simultaneously with the purified RhD antigen at 20 μg/ml and used to stimulate PBMC. Proliferative responses were measured five days after stimulation. A decrease in SI value of more than 1.8 was interpreted as representing a significant inhibition of the proliferative response to the RhD protein (after Devereux et al (2000) J. Imm. Meth. 234; 13-22. At the same time, control wells contained PBMC stimulated with RhD antigen or the IL-10 inducing peptide alone. Results from the two representative donors are shown in FIGS. 13A and 13B.

To ensure that any inhibition or suppression observed was due to IL-10 elicitation, parallel cultures were made to which 2000 pg/ml of dialysed rat anti-human IL-10 Mab (Pharmingen, San Diego, USA) was added. Proliferative responses were measured five days after stimulation and it was noted that there was a reversal of the inhibitory response by IL-10 inducing peptides. Results from three representative donors are shown in FIGS. 14A to 14C.

These experiments demonstrate that it is possible to use IL-10 inducing RhD peptides to suppress the effector immune response to the RhD protein in vivo. The addition of such peptides to T-cell cultures specifically blocks the proliferative response to the RhD protein, but not to a control antigen PPD. Accordingly, compositions of the present invention may be able to inhibit damaging responses in vivo if given to patients, whilst not suppressing the rest of the immune system.

### EXAMPLE 1.6

A 68 RhD peptide panel was produced in the same manner as Example 1 except the Met 218 version of the RhD protein was used.

RhD Immunisation Schedule

HLA-DR15 transgenic mice were immunised twice with 2 mg/ml purified RhD protein. The first subcutaneous injection was followed 14 days later by an intraperitoneal injection. Mice were sacrificed 14 days after the final injection. The spleen was removed and blood collected by cardiac puncture.

Tolerisation Schedule

Mucosal Route

Fourteen days before RhD immunisation (as above) 25 μl of 2 mg/ml soluble RhD peptides were administered by the intranasal route.

Parenteral Route

Fourteen days before RhD immunisation (as above) 25 μl of 2 mg/ml soluble RhD peptides were administered by the subcutaneous route.

T-Cell Proliferation Assay

Splenocytes were homogenised and a single cell suspension was isolated over 40 μm mesh filters (Becton Dickinson) and resuspended to 1.25×10⁶ cells/ml in the Alpha Modification of Eagle's Medium (ICN Flow, Bucks UK) supplemented with 5% autologous serum, 4 mM L-Glutamine (Gibco, Paisley, UK), 100 U/ml sodium benzylpenicillin G (Sigma), 100 μg/ml streptomycin sulphate (Sigma), 5×10⁻⁵ 2-mercaptoethanol (Sigma) and 20 mM HEPES pH7.2 (Sigma). All plates were incubated at 37° C. in a humidified atmosphere of 5% CO₂/95% air. The cell proliferation in cultures was estimated from the incorporation of ³H-thymidine (Amersham) in triplicate wells 5 days after stimulation with antigen. Proliferation results are presented either as the mean CPM±SD of the triplicate samples, or as a stimulation index (SI) expressing the ratio of mean CPM in stimulated versus unstimulated control cultures. An SI>3 with CPM>500 is interpreted as representing a positive response.

Serum Anti-Human Antibody Quantification

Round bottomed microtitre plates (Nunc, Roskilde, Denmark) were blocked in phosphate buffered saline pH 7.4 (PBS) containing 0.2% bovine serum albumin (BSA), before addition of 50 μl 2% v/v washed human D-positive RBC. Test sera were incubated at 50 μl per well with triplicate RBC samples for 1 hour at 37° C. and washed three times in PBS-BSA before fixing for 30 minutes in 0.15% glutaraldehyde (Sigma, Dorset, UK) to prevent lysis of the cells in the alkaline conditions required later in the test. The fixed RBC were transferred to fresh, pre-blocked, 96-well plates and washed before incubation with 50 μl per well 1 μg/ml goat anti-mouse IgG γ-chain specific antibody (Sigma) or goat anti-mouse IgM μ-chain specific antibody (Sigma) for 1 hour at 37° C. After washing, the plates were incubated with 50 μl per well 1 μg/ml rabbit anti-goat IgG alkaline phosphatase (Sigma) for 1 hour at 37° C., washed, and 100 μl of phosphatase substrate solution was then incubated in each well for 1 hour at 37° C. After pelleting of the RBC by centrifugation, 50 μl of each supernatant was transferred into the wells of fresh, flat-bottomed microtitre plates (Nunc) and the absorbance measured at 405 nm, with 492 nm as a reference, using a multiscan plate reader (Labsystems, Basingstoke, UK). Each result is expressed as the mean of triplicate wells. Inter-assay variation was controlled for by including previously tested RBC samples on each plate.

It is known from other animal models (Elson C J & Barker R N. Helper T-cells in antibody mediated, organ specific autoimmunity. Curr Opin Immunol, 2000; 12:664-669) that mucosal administration of immunodominant peptides which induce an immune response when given with adjuvant by the parenteral route will inhibit responsiveness, and induce antigen specific tolerance. To test the effectiveness of peptides containing immunodominant allo-RhD helper epitopes in preventing antibody response to the RhD protein, HLA-DR15 transgenic mice were used for the in vivo testing of these peptides as most alloimmunised donors responding to RhD bear this MHC class II allele.

DR15 mice were immunised with RhD protein and it was shown that this schedule produced a specific memory T-cell proliferative response to the RhD protein and serum antibodies, which are specific for human RhD-positive red blood cells (R₂R₂ phenotype).

Four of the immunodominant epitopes of RhD identified in alloimmunised donors (SEQ ID Nos: 6, 13, 17 and 28) were tested for the induction of nasal tolerance in the HLA-DR15 transgenic mouse model. Each was capable of inducing tolerance as demonstrated by a reduction in the RhD-specific proliferation by splenocytes from RhD immunised mice that had been previously tolerised via the nasal mucosa with peptide 6, 13, 17 and 28. An illustrative example of the induction of tolerance to RhD immunisation by peptide 6 is shown in FIG. 15.

In most cases proliferation was associated with both IFN-γ (6/9, Rs>0.28, p<0.03) and IL-4 production (5/9, Rs>0.31, p<0.04) indicating a Th0 response, and serum antibodies from immunised mice agglutinated human erythrocytes. Splenocytes from control mice immunised with PBS rarely induced proliferation (2/12) in response to stimulation with purified RhD protein or peptides, and did not produce antibodies against human red cells. Mucosal administration of immunodominant RhD peptides prior to immunisation with RhD protein was found to inhibit the proliferative response to RhD protein and markedly reduce the levels of serum antibodies. Unlike splenocytes, cells from lymph nodes draining the site of RhD protein immunisation produce the Tr1 and Th3 cytokines IL-10 and TGF-1, respectively, in response to stimulation with both the tolerising RhD peptide and RhD protein (FIG. 16). These results provide evidence that tolerance can be achieved by mucosal delivery of dominant peptides from the RhD protein and will, therefore, be of value in the prevention of immunisation in RhD-negative individuals.

### EXAMPLE 2

### Relating to Therapy for Autoimmune Haemolytic Anaemia

### EXAMPLE 2.1

The experiments mentioned in Example 1.1 were repeated using blood from subjects suffering from autoimmune haemolytic anaemia. It was therefore established that the T-cells from many of the subjects exhibited a proliferative response to peptides 2, 3, 20, 33, 47, 50 and 62 (see FIG. 17A).

These experiments can be carried out using other rhesus proteins, such as RhcE, Rhce, RhCe or RhCE protein. T-cells from RhD-negative subjects suffering AIHA were tested with the panel of ce peptides (Table 3). It was therefore established that the T-cells from many of the subjects exhibited a proliferative response to peptides 143, 144, 168, 186 and 195 (see FIG. 17B)

Responses to the RhD peptide panel were rare in both RhD-positive (FIG. 18) and RhD-negative healthy (FIG. 7) control, unimmunised donors.

### EXAMPLE 2.2

The experiments mentioned in Example 1.2 were repeated using blood from subjects suffering from autoimmune haemolytic anaemia. For warm-type autoimmune haemolytic anaemia there is an association with HLA DR15 with 65% of patients carrying this HLA type (see FIG. 19A). It is likely therefore, that HLA-DRB1*15 preferentially enhances the likelihood of responses to epitopes from the RhD protein.

The group of patients with AIHA tested in Example 2.1 was divided into two groups; those that express HLA-DRB1*1501 and those that do not; and the distribution of stimulatory epitopes was calculated. RhD peptides are more likely to induce proliferative responses by PBMC from HLA-DR15+AIHA patients compared to patients that do not express HLA-DR15. (see FIGS. 19B and 19C). Thus carrying this tissue type is associated with an increased risk of producing anti-D antibodies after exposure to RhD positive erythrocytes.

The broad spectrum of stimulatory epitopes, from the RhD protein, between alloimmunised individuals could reflect the different HLA class II molecules expressed by antigen presenting cells. Two donors were matched for HLA-DR and HLA-DQ type; specifically HLA-DRB1*1501 and HLA-DQB1*11; and the response profile of stimulatory RhD peptides compared (see FIG. 19). It was found that there is a strong positive correlation between these individuals (Rₛ=0.515, p<0.001).

A statistical analysis of all the data shows that the effect of HLA-DR type on the identity of the peptides recognised is relatively weak. In other words, many of the RhD peptides stimulate T-cells regardless of tissue type. For example, at least one of the four RhD peptides that are most likely to induce proliferation responses will induce a response by T-cells from all AIHA patients tested.

These analyses demonstrate that the selection of RhD peptide fragments for immunisation/tolerisation regimes may not be dependent on prior tissue typing of recipients, an important practical consideration for the clinical application of this approach.

### EXAMPLE 2.3

The following Example identifies the RhD peptides that induce regulatory responses to the RhD protein in autoimmune haemolytic anaemia patients.

The same 68 RhD peptide panel as used in Example 1.3 was used in the present Example. Further, the same experimental protocols used in Examples 1.3.

Using the celELISA technique, 42 of the 68 RhD peptide panel were screened for the ability to induce IL-10 production from PBMC from 11 patients with AIHA. The RhD peptides that induced significant IL-10 responses from PBMC in all the samples screened are summarised in FIG. 21.

An analysis of the proportion of AIHA patients with PBMC that mounted significant responses to each of the 42 RhD peptides tested revealed that the following peptides stimulated PBMC from >20% of the patients to produce significant IL-10:
2, 3, 5, 6, 8, 12, 14, 16, 21, 23, 25, 27, 35, 37, 39, 41, 44, 54, 56, 60, 62, 63

More than 40% of donors produced significant IL-10 in response to peptides:
3, 6, 14, 35, 37, 54, 56, 63.

In contrast, significant TGF-β1 production by PBMC from AIHA patients was rarely detected (FIG. 22).

Therefore, it is apparent that the RhD peptides can induce the production of significant levels of IL-10 from PBMC isolated from AIHA patients. PBMC from RhD-positive control donors with no known exposure to RhD-positive RBC produce very few proliferative responses (FIG. 18), but can produce significant IL-10 cytokine responses to the RhD peptides (data not shown).

### EXAMPLE 2.4

The secretion of cytokines identified in Example 2.3 by T-cells is confirmed utilising the experiments mentioned in Example 1.4, which were repeated using PBMC isolated from patients with AIHA.

Stimulation of PBMC from an AIHA patient with RhD protein and RhD peptides 35 and 60, as per Table 1, is associated with the expansion of a CD3+ CD4+population of T-helper cells (FIGS. 23A to 23D). Furthermore, the proliferative response is associated with the expansion of CD4+ T-helper cells that express intracellular IFN-γ (FIGS. 23E to 23H). This confirms that the population of cells expanding following stimulation with RhD protein and peptides are T helper cells with a Th1 bias.

Stimulation of PBMC from an AIHA patient with the IL-10 inducing RhD peptide P36 induces the production of IL-10 (FIG. 24A). This is associated with the expansion of a CD3+ CD4+population of T helper cells (FIGS. 24B and 24C) that express intracellular IL-10 (FIGS. 24D and 24E). These results confirm that IL-10 detected by celELISA is generated by T-helper cells.

These results confirm that T-helper cells are the primary source of IFN-γ and IL-10 when PBMC from AIHA patients are stimulated with RhD.

### EXAMPLE 2.5

This Example shows that IL-10 inducing peptides can specifically inhibit T-cell responses to RhD protein. The same experimental protocol was used as laid out in Example 1.5.

The RhD peptides identified in Example 2.3, that elicit IL-10 responses by PBMCs from AIHA patients were added to respective PBMC cultures that had been stimulated to proliferate by purified RhD protein. The percentage inhibition of proliferation is shown in representative experiments (FIGS. 25A and 25B) using samples from 2 patients. Importantly, inhibition appears to be specific for the RhD protein since the proliferative response to the recall antigen, PPD, was not inhibited. Similar results were obtained in a total of 8 experiments, using samples from 3 patients, and testing at least 2 IL-10 inducing peptides on each occasion.

The dependence of inhibition on IL-10 production was confirmed with an anti-IL-10 neutralising antibody, which inhibited or completely abrogated the peptide induced inhibition of the proliferative response (FIG. 26).

This data demonstrates that it is possible to use IL-10 inducing peptides to suppress the autoimmune response to Rh related peptides in ‘warm’ AIHA patients.

### EXAMPLE 2.6

This Example shows that IL-10 inducing peptides will induce tolerance to the RhD protein when administered by the parenteral route without adjuvant (i.e. in a non-immunogenic form).

The same experimental protocol as set out in Example 1.6 was used.

The experimental results in FIGS. 27A to 27C show that DR15 mice produced a specific memory T-cell proliferative response to the RhD protein and serum antibodies, which are specific for human RhD-positive red blood cells (R2R2 phenotype).

IL-10 inducing RhD peptides were capable of inducing tolerance as demonstrated by a reduction in the RhD-specific proliferation by splenocytes from mice tolerised via the parenteral route with IL-10 inducing peptides, given before immunisation with RhD. Antibodies specific for RhD-bearing red blood cells, R2R2, could also be significantly reduced after parenteral administration of such peptides, given before immunisation with RhD.

TABLE 1

| | |
|---|---|
| | SEQ |
| RhD PEPTIDE | ID |

| | | | |
|---|---|---|---|
| SEQUENCE | No. | RESIDUES | FOLDING |

| | | | | |
|---|---|---|---|---|
| SSKYPRSVRRCLPLW | 1 | 2-16 | 2-12 | Internal —NH4 |
| CLPLWALTLEAALIL | 2 | 12-26 | 13-29 | Transmem- |
| | | | | brane 1 |
| AALILLFYFFTHYDA | 3 | 22-36 | | |
| THYDASLEDQKGLVA | 4 | 32-46 | 30-52 | External |
| | | | | loop 1 |
| KGLVASYQVGQDLTV | 5 | 42-56 | | |
| QDLTVMAAIGLGFLT | 6 | 52-66 | 52-70 | Transmem- |
| | | | | brane 2 |
| MAAIGLGFLTSSFRR | 7 | 57-71 | | |
| LGFLTSSFRRHSWSS | 8 | 62-76 | | |
| SSFRRHSWSSVAFNL | 9 | 67-81 | 71-75 | Internal |
| | | | | Loop 1 |
| HSWSSVAFNLFMLAL | 10 | 72-86 | | |
| FMLALGVQWAILLDG | 11 | 82-96 | 76-92 | Transmem- |
| | | | | brane 3 |
| ILLDGFLSQFPSGKV | 12 | 92-106 | | |
| FLSQFPSGKVVITLF | 13 | 97-111 | 93-109 | External |
| | | | | Loop 2 |
| PSGKVVITLFSIRLA | 14 | 102-116 | | |
| VITLFSIRLATMSAL | 15 | 107-121 | | |
| SIRLATMSALSVLIS | 16 | 112-126 | 110-129 | Transmem- |
| | | | | brane 4 |
| TMSALSVLISVDAVL | 17 | 117-131 | | |
| SVLISVDAVLGKVNL | 18 | 122-136 | 130-134 | Internal |
| | | | | Loop 2 |
| VDAVLGKVNLAQLVV | 19 | 127-141 | | |
| GKVNLAQLVVMVLVE | 20 | 132-146 | 135-152 | Transmem- |
| | | | | brane 5 |
| MVLVEVTALGNLRMV | 21 | 142-156 | | |
| VTALGNLRMVISNIF | 22 | 147-161 | | |
| NLRMVISNIFNTDYH | 23 | 152-166 | 153-168 | External |
| | | | | Loop 3 |
| ISNIFNTDYHMNMMH | 24 | 157-171 | | |
| NTDYHMNMMHIYVFA | 25 | 162-176 | | |
| MNMMHIYVFAAYFGL | 26 | 167-181 | 169-186 | Transmem- |
| | | | | brane 6 |
| IYVFAAYFGLSVAWC | 27 | 172-186 | | |
| AYFGLSVAWCLPKPL | 28 | 177-191 | | |
| SVAWCLPKPLPEGTE | 29 | 182-196 | 187-211 | Internal |
| | | | | Loop 3 |
| LPKPLPEGTEDKDQT | 30 | 187-201 | | |
| PEGTEDKDQTATIPS | 31 | 192-206 | | |
| DKDQTATIPSLSAML | 32 | 197-211 | | |
| ATIPSLSAMLGALFL | 33 | 202-216 | | |
| LSAMLGALFLWMFWP | 34 | 207-221 | | |
| GALFLWMFWPSFNSA | 35 | 212-226 | 212-229 | Transmem- |
| | | | | brane 7 |
| WMFWPSFNSALLRSP | 36 | 217-231 | | |
| SFNSALLRSPIERKN | 37 | 222-236 | | |
| LLRSPIERKNAVFNT | 38 | 227-241 | 230-241 | External |
| | | | | Loop 4 |
| IERKNAVFNTYYALA | 39 | 232-246 | | |
| AVFNTYYAVAVSVVT | 40 | 237-251 | | |
| YYAVAVSVVTAISGS | 41 | 242-256 | 242-259 | Transmem- |
| | | | | brane 8 |
| AISGSSLAHPQGKIS | 42 | 252-266 | | |
| SLAHPQGKISKTYVH | 43 | 257-271 | 260-267 | Internal |
| | | | | Loop 4 |
| QGKISKTYVHSAVLA | 44 | 262-276 | | |
| KTYVHSAVLAGGVAV | 45 | 267-281 | 268-285 | Transmem- |
| | | | | brane 9 |
| SAVLAGGVAVGTSCH | 46 | 272-286 | | |
| GTSCHLIPSPWLAMV | 47 | 282-296 | 286-290 | External |
| | | | | Loop 5 |
| WLAMVLGLVAGLISV | 48 | 292-306 | 291-308 | Transmem- |
| | | | | brane 10 |
| LGLVAGLISVGGAKY | 49 | 297-311 | | |
| GLISVGGAKYLPGCC | 50 | 302-316 | | |
| GGAKYLPGCCNRVLG | 51 | 307-321 | 309-335 | Internal |
| | | | | Loop 5 |
| LPGCCNRVLGIPHSS | 52 | 312-326 | | |
| NRVLGIPHSSIMGYN | 53 | 317-331 | | |
| IPHSSIMGYNFSLLG | 54 | 322-336 | | |
| IMGYNFSLLGLLGEI | 55 | 327-341 | | |
| FSLLGLLGEIIYIVL | 56 | 332-346 | 336-352 | Transmem- |
| | | | | brane 11 |
| LLGEIIYIVLLVLDT | 57 | 337-351 | | |
| IYIVLLVLDTVGAGN | 58 | 342-356 | | |
| LVLDTVGAGNGMIGF | 59 | 347-361 | | |
| VGAGNGMIGFQVLLS | 60 | 352-366 | 353-371 | External |
| | | | | Loop 6 |
| QVLLSIGELSLAIVI | 61 | 362-376 | | |
| LAIVIALTSGLLTGL | 62 | 372-386 | 372-388 | Transmem- |
| | | | | brane 12 |
| LLTGLLLNLKIWKAP | 63 | 382-396 | | |
| LLNLKIWKAPHEAKY | 64 | 387-401 | 389-417 | Internal —COOH |
| IWKAPHEAKYFDDQV | 65 | 392-406 | | |
| HEAKYFDDQVFWKFP | 66 | 397-411 | | |
| FDDQVFWKFPHLAVG | 67 | 402-416 | | |
| DDQVFWKFPHLAVGF | 68 | 403-417 | | |
| LSAMLGALFLWIFWP* | 69 | 207-221 | | |
| GALFLWIFWPSFNS* | 70 | 212-226 | 212-229 | Transmem- |
| | | | | brane 7 |
| WIFWPSFNSALLRSP* | 71 | 217-231 | | |

*Published sequences differ at position 218. The sequence we originally submitted was the more widely cited¹ variant Ile218², which is now thought to be a sequencing error³, with Met218⁴ now generally accepted as the correct residue.
¹Reid ME, Lomas Francis C. Rh blood group system. In: The Blood Group Antigen Facts Book. Academic Press. 1996, p94.
²Le Van Kim C, Mouro I, Chérif-Zahar B, Raynal V. Cherrier C, Cartron J-P, Colin Y. Molecular cloning and primary structure of the human blood group RhD polypeptide. Proc. Natl. Acad. Sci. USA. 1992;89:10925-10929.
³Cartron J-P, Rouillac C, Le Van Kim C, Mouro I, Colin Y. Tentative model for the mapping of D epitopes on the RhD polypeptide. Transfusion Clinique et Biologique. 1996,6:497-503.
⁴Arce MA, Thompson ES, Wagner 5, Coyne KE, Ferdman BA, Lublin DM. Molecular cloning of RhD cDNA derived from a gene present in RhD-positive, but not RhD-negative individuals. Blood. 1993; 82:651-655.

TABLE 2

| | | | |
|---|---|---|---|
| | SEQ | | |
| RhcE PEPTIDE | ID | | |
| SEQUENCE | No. | RESIDUES | FOLDING |

| | | | | |
|---|---|---|---|---|
| SSKYPRSVRRCLPLW | 72 | 2-16 | 2-12 | Internal —NH4 |
| CLPLWALTLEAALIL | 73 | 12-26 | 13-29 | Transmem- |
| | | | | brane 1 |
| AALILLFYFFTHYDA | 74 | 22-36 | | |
| THYDASLEDQKGLVA | 75 | 32-46 | 30-52 | External |
| | | | | loop 1 |
| KGLVASYQVGQDLTV | 76 | 42-56 | | |
| QDLTVMAALGLGFLT | 77 | 52-66 | 52-70 | Transmem- |
| | | | | brane 2 |
| MAALGLGFLTSNFRR | 78 | 57-71 | | |
| LGFLTSNFRRHSWSS | 79 | 62-76 | | |
| SNFRRHSWSSVAFNL | 80 | 67-81 | 71-75 | Internal |
| | | | | Loop 1 |
| HSWSSVAFNLFMLAL | 81 | 72-86 | | |
| FMLALGVQWAILLDG | 82 | 82-96 | 76-92 | Transmem- |
| | | | | brane 3 |
| ILLDGFLSQFPPGKV | 83 | 92-106 | | |
| FLSQFPPGKVVITLF | 84 | 97-111 | 93-109 | External |
| | | | | Loop 2 |
| PPGKVVITLFSIRLA | 85 | 102-116 | | |
| VITLFSIRLATMSAM | 86 | 107-121 | | |
| SIRLATMSAMSVLIS | 87 | 112-126 | 110-129 | Transmem- |
| | | | | brane 4 |
| TMSAMSVLISAGAVL | 88 | 117-131 | | |
| SVLISAGAVLGKVNL | 89 | 122-136 | 130-134 | Internal |
| | | | | Loop 2 |
| AGAVLGKVNLAQLVV | 90 | 127-141 | | |
| GKVNLAQLVVMVLVE | 91 | 132-146 | 135-152 | Transmem- |
| | | | | brane 5 |
| MVLVEVTALGTLRMV | 92 | 142-156 | | |
| VTALGTLRMVISNIF | 93 | 147-161 | | |
| TLRMVISNIFNTDYH | 94 | 152-166 | 153-168 | External |
| | | | | Loop 3 |
| ISNIFNTDYHMNLRH | 95 | 157-171 | | |
| NTDYHMNLRHIYVFA | 96 | 162-176 | | |
| MNLRHIYVFAAYFGL | 97 | 167-181 | 169-186 | Transmem- |
| | | | | brane 6 |
| IYVFAAYFGLTVAWC | 98 | 172-186 | | |
| AYFGLTVAWCLPKPL | 99 | 177-191 | | |
| TVAWCLPKPLPKGTE | 100 | 182-196 | 187-211 | Internal |
| | | | | Loop 3 |
| LPKPLKEGTEDKDQR | 101 | 187-201 | | |
| PKGTEDNDQRATIPS | 102 | 192-206 | | |
| DNDQRATIPSLSAML | 103 | 197-211 | | |
| ATIPSLSAMLGALFL | 104 | 202-216 | | |
| LSAMLGALFLWMFWP | 105 | 207-221 | | |
| GALFLWMFWPSVNSP | 106 | 212-226 | 212-229 | Transmem- |
| | | | | brane 7 |
| WMFWPSVNSPLLRSP | 107 | 217-231 | | |
| SVNSPLLRSPIQRKN | 108 | 222-236 | | |
| LLRSPIQRKNAMFNT | 109 | 227-241 | 230-241 | External |
| | | | | Loop 4 |
| IQRKNAMFNTYYALA | 110 | 232-246 | | |
| AMFNTYYALAVSVVT | 111 | 237-251 | | |
| YYAVAVSVVTAISGS | 112 | 242-256 | 242-259 | Transmem- |
| | | | | brane 8 |
| AISGSSLAHPQRKIS | 113 | 252-266 | | |
| SLAHPQRKISMTYVH | 114 | 257-271 | 260-267 | Internal |
| | | | | Loop 4 |
| QRKISMTYVHSAVLA | 115 | 262-276 | | |
| MTYVHSAVLAGGVAV | 116 | 267-281 | 268-285 | Transmem- |
| | | | | brane 9 |
| SAVLAGGVAVGTSCH | 117 | 272-286 | | |
| GTSCHLIPSPWLAMV | 118 | 282-296 | 286-290 | External |
| | | | | Loop 5 |
| WLAMVLGLVAGLISI | 119 | 292-306 | 291-308 | Transmem- |
| | | | | brane 10 |
| LGLVAGLISIGGAKY | 120 | 297-311 | | |
| GLISIGGAKCLPVCC | 121 | 302-316 | | |
| GGAKCLPVCCNRVLG | 122 | 307-321 | 309-335 | Internal |
| | | | | Loop 5 |
| LPVCCNRVLGIHHIS | 123 | 312-326 | | |
| NRVLGIHHISVMHSI | 124 | 317-331 | | |
| IHHISVMHSIFSLLG | 125 | 322-336 | | |
| IMHSIFSLLGLLGEI | 126 | 327-341 | | |
| FSLLGLLGEITYIVL | 127 | 332-346 | 336-352 | Transmem- |
| | | | | brane 11 |
| LLGEITYIVLLVLHT | 128 | 337-351 | | |
| TYIVLLVLHTVWNGN | 129 | 342-356 | | |
| LVLHTVWNGNGMIGF | 130 | 347-361 | | |
| VWNGNGMIGFQVLLS | 131 | 352-366 | 353-371 | External |
| | | | | Loop 6 |
| QVLLSIGELSLAIVI | 132 | 362-376 | | |
| LAIVIALTSGLLTGL | 133 | 372-386 | 372-388 | Transmem- |
| | | | | brane 12 |
| LLTGLLLNLKIWKAP | 134 | 382-396 | | |
| LLNLKIWKAPHVAKY | 135 | 387-401 | 389-417 | Internal |
| | | | | —COOH |
| IWKAPHVAKYFDDQV | 136 | 392-406 | | |
| HVAKYFDDQVFWKFP | 137 | 397-411 | | |
| FDDQVFWKFPHLAVG | 138 | 402-416 | | |
| DDQVFWKFPHLAVGF | 139 | 403-417 | | |

TABLE 3

| | | | |
|---|---|---|---|
| | SEQ | | |
| Rhce PEPTIDE | ID | | |
| SEQUENCE | No. | RESIDUES | FOLDING |

| | | | | |
|---|---|---|---|---|
| SSKYPRSVRRCLPLW | 140 | 2-16 | 2-12 | Internal —NH4 |
| CLPLWALTLEAALIL | 141 | 12-26 | 13-29 | Transmem- |
| | | | | brane 1 |
| AALILLFYFFTHYDA | 142 | 22-36 | | |
| THYDASLEDQKGLVA | 143 | 32-46 | 30-52 | External |
| | | | | loop 1 |
| KGLVASYQVGQDLTV | 144 | 42-56 | | |
| QDLTVMAALGLGFLT | 145 | 52-66 | 52-70 | Transmem- |
| | | | | brane 2 |
| MAALGLGFLTSNFRR | 146 | 57-71 | | |
| LGFLTSNFRRHSWSS | 147 | 62-76 | | |
| SNFRRHSWSSVAFNL | 148 | 67-81 | 71-75 | Internal |
| | | | | Loop 1 |
| HSWSSVAFNLFMLAL | 149 | 72-86 | | |
| FMLALGVQWAILLDG | 150 | 82-96 | 76-92 | Transmem- |
| | | | | brane 3 |
| ILLDGFLSQFPPGKV | 151 | 92-106 | | |
| FLSQFPPGKVVITLF | 152 | 97-111 | 93-109 | External |
| | | | | Loop 2 |
| PPGKVVITLFSIRLA | 153 | 102-116 | | |
| VITLFSIRLATMSAM | 154 | 107-121 | | |
| SIRLATMSAMSVLIS | 155 | 112-126 | 110-129 | Transmem- |
| | | | | brane 4 |
| TMSAMSVLISAGAVL | 156 | 117-131 | | |
| SVLISAGAVLGKVNL | 157 | 122-136 | 130-134 | Internal |
| | | | | Loop 2 |
| AGAVLGKVNLAQLVV | 158 | 127-141 | | |
| GKVNLAQLVVMVLVE | 159 | 132-146 | 135-152 | Transmem- |
| | | | | brane 5 |
| MVLVEVTALGTLRMV | 160 | 142-156 | | |
| VTALGTLRMVISNIF | 161 | 147-161 | | |
| TLRMVISNIFNTDYH | 162 | 152-166 | 153-168 | External |
| | | | | Loop 3 |
| ISNIFNTDYHMNLRH | 163 | 157-171 | | |
| NTDYHMNLRHIYVFA | 164 | 162-176 | | |
| MNLRHIYVFAAYFGL | 165 | 167-181 | 169-186 | Transmem- |
| | | | | brane 6 |
| IYVFAAYFGLTVAWC | 166 | 172-186 | | |
| AYFGLTVAWCLPKPL | 167 | 177-191 | | |
| TVAWCLPKPLPKGTE | 168 | 182-196 | 187-211 | Internal |
| | | | | Loop 3 |
| LPKPLKEGTEDKDQR | 169 | 187-201 | | |
| PKGTEDNDQRATIPS | 170 | 192-206 | | |
| DNDQRATIPSLSAML | 171 | 197-211 | | |
| ATIPSLSAMLGALFL | 172 | 202-216 | | |
| LSAMLGALFLWMFWP | 173 | 207-221 | | |
| GALFLWMFWPSVNSA | 174 | 212-226 | 212-229 | Transmem- |
| | | | | brane 7 |
| WMFWPSVNSALLRSP | 175 | 217-231 | | |
| SVNAPLLRSPIQRKN | 176 | 222-236 | | |
| LLRSPIQRKNAMFNT | 177 | 227-241 | 230-241 | External |
| | | | | Loop 4 |
| IQRKNAMFNTYYALA | 178 | 232-246 | | |
| AMFNTYYALAVSVVT | 179 | 237-251 | | |
| YYAVAVSVVTAISGS | 180 | 242-256 | 242-259 | Transmem- |
| | | | | brane 8 |
| AISGSSLAHPQRKIS | 181 | 252-266 | | |
| SLAHPQRKISMTYVH | 182 | 257-271 | 260-267 | Internal |
| | | | | Loop 4 |
| QRKISMTYVHSAVLA | 183 | 262-276 | | |
| MTYVHSAVLAGGVAV | 184 | 267-281 | 268-285 | Transmem- |
| | | | | brane 9 |
| SAVLAGGVAVGTSCH | 185 | 272-286 | | |
| GTSCHLIPSPWLAMV | 186 | 282-296 | 286-290 | External |
| | | | | Loop 5 |
| WLAMVLGLVAGLISI | 187 | 292-306 | 291-308 | Transmem- |
| | | | | brane 10 |
| LGLVAGLISIGGAKY | 188 | 297-311 | | |
| GLISIGGAKCLPVCC | 189 | 302-316 | | |
| GGAKCLPVCCNRVLG | 190 | 307-321 | 309-335 | Internal |
| | | | | Loop 5 |
| LPVCCNRVLGIHHIS | 191 | 312-326 | | |
| NRVLGIHHISVMHSI | 192 | 317-331 | | |
| IHHISVMHSIFSLLG | 193 | 322-336 | | |
| IMHSIFSLLGLLGEI | 194 | 327-341 | | |
| FSLLGLLGEITYIVL | 195 | 332-346 | 336-352 | Transmem- |
| | | | | brane 11 |
| LLGEITYIVLLVLHT | 196 | 337-351 | | |
| TYIVLLVLHTVWNGN | 197 | 342-356 | | |
| LVLHTVWNGNGMIGF | 198 | 347-361 | | |
| VWNGNGMIGFQVLLS | 199 | 352-366 | 353-371 | External |
| | | | | Loop 6 |
| QVLLSIGELSLAIVI | 200 | 362-376 | | |
| LAIVIALTSGLLTGL | 201 | 372-386 | 372-388 | Transmem- |
| | | | | brane 12 |
| LLTGLLLNLKIWKAP | 202 | 382-396 | | |
| LLNLKIWKAPHVAKY | 203 | 387-401 | 389-417 | Internal |
| | | | | —COOH |
| IWKAPHVAKYFDDQV | 204 | 392-406 | | |
| HVAKYFDDQVFWKFP | 205 | 397-411 | | |
| FDDQVFWKFPHLAVG | 206 | 402-416 | | |
| DDQVFWKFPHLAVGF | 207 | 403-417 | | |

TABLE 4

| | | | |
|---|---|---|---|
| | SEQ | | |
| RhCe PEPTIDE | ID | | |
| SEQUENCE | No. | RESIDUES | FOLDING |

| | | | | |
|---|---|---|---|---|
| SSKYPRSVRRCLPLC | 208 | 2-16 | 2-12 | Internal —NH4 |
| CLPLCALTLEAALIL | 209 | 12-26 | 13-29 | Transmem- |
| | | | | brane 1 |
| AALILLFYFFTHYDA | 210 | 22-36 | | |
| THYDASLEDQKGLVA | 211 | 32-46 | 30-52 | External |
| | | | | loop 1 |
| KGLVASYQVGQDLTV | 212 | 42-56 | | |
| QDLTVMAAIGLGFLT | 213 | 52-66 | 52-70 | Transmem- |
| | | | | brane 2 |
| MAAIGLGFLTSNSRR | 214 | 57-71 | | |
| LGFLTSSFRRHSWSS | 215 | 62-76 | | |
| SSFRRHSWSSVAFNL | 216 | 67-81 | 71-75 | Internal |
| | | | | Loop 1 |
| HSWSSVAFNLFMLAL | 217 | 72-86 | | |
| FMLALGVQWAILLDG | 218 | 82-96 | 76-92 | Transmem- |
| | | | | brane 3 |
| ILLDGFLSQFPSGKV | 219 | 92-106 | | |
| FLSQFPSGKVVITLF | 220 | 97-111 | 93-109 | External |
| | | | | Loop 2 |
| PSGKVVITLFSIRLA | 221 | 102-116 | | |
| VITLFSIRLATMSAM | 222 | 107-121 | | |
| SIRLATMSAMSVLIS | 223 | 112-126 | 110-129 | Transmem- |
| | | | | brane 4 |
| TMSAMSVLISAGAVL | 224 | 117-131 | | |
| SVLISAGAVLGKVNL | 225 | 122-136 | 130-134 | Internal |
| | | | | Loop 2 |
| AGAVLGKVNLAQLVV | 226 | 127-141 | | |
| GKVNLAQLVVMVLVE | 227 | 132-146 | 135-152 | Transmem- |
| | | | | brane 5 |
| MVLVEVTALGTLRMV | 228 | 142-156 | | |
| VTALGTLRMVISNIF | 229 | 147-161 | | |
| TLRMVISNIFNTDYH | 230 | 152-166 | 153-168 | External |
| | | | | Loop 3 |
| ISNIFNTDYHMNLRH | 231 | 157-171 | | |
| NTDYHMNLRHIYVFA | 232 | 162-176 | | |
| MNLRHIYVFAAYFGL | 233 | 167-181 | 169-186 | Transmem- |
| | | | | brane 6 |
| IYVFAAYFGLTVAWC | 234 | 172-186 | | |
| AYFGLTVAWCLPKPL | 235 | 177-191 | | |
| TVAWCLPKPLPKGTE | 236 | 182-196 | 187-211 | Internal |
| | | | | Loop 3 |
| LPKPLKEGTEDKDQR | 237 | 187-201 | | |
| PKGTEDNDQRATIPS | 238 | 192-206 | | |
| DNDQRATIPSLSAML | 239 | 197-211 | | |
| ATIPSLSAMLGALFL | 240 | 202-216 | | |
| LSAMLGALFLWMFWP | 241 | 207-221 | | |
| GALFLWMFWPSVNSA | 242 | 212-226 | 212-229 | Transmem- |
| | | | | brane 7 |
| WMFWPSVNSALLRSP | 243 | 217-231 | | |
| SVNSALLRSPIQRKN | 244 | 222-236 | | |
| LLRSPIQRKNAMFNT | 245 | 227-241 | 230-241 | External |
| | | | | Loop 4 |
| IQRKNAMFNTYYALA | 246 | 232-246 | | |
| AMFNTYYALLAVSVVT | 247 | 237-251 | | |
| YYAVAVSVVTAISGS | 248 | 242-256 | 242-259 | Transmem- |
| | | | | brane 8 |
| AISGSSLAHPQRKIS | 249 | 252-266 | | |
| SLAHPQRKISMTYVH | 250 | 257-271 | 260-267 | Internal |
| | | | | Loop 4 |
| QRKISMTYVHSAVLA | 251 | 262-276 | | |
| MTYVHSAVLAGGVAV | 252 | 267-281 | 268-285 | Transmem- |
| | | | | brane 9 |
| SAVLAGGVAVGTSCH | 253 | 272-286 | | |
| GTSCHLIPSPWLAMV | 254 | 282-296 | 286-290 | External |
| | | | | Loop 5 |
| WLAMVLGLVAGLISI | 255 | 292-306 | 291-308 | Transmem- |
| | | | | brane 10 |
| LGLVAGLISIGGAKY | 256 | 297-311 | | |
| GLISIGGAKCLPVCC | 257 | 302-316 | | |
| GGAKCLPVCCNRVLG | 258 | 307-321 | 309-335 | Internal |
| | | | | Loop 5 |
| LPVCCNRVLGIHHIS | 259 | 312-326 | | |
| NRVLGIHHISVMHSI | 260 | 317-331 | | |
| IHHISVMHSIFSLLG | 261 | 322-336 | | |
| IMHSIFSLLGLLGEI | 262 | 327-341 | | |
| FSLLGLLGEITYIVL | 263 | 332-346 | 336-352 | Transmem- |
| | | | | brane 11 |
| LLGEITYIVLLVLHT | 264 | 337-351 | | |
| TYIVLLVLHTVWNGN | 265 | 342-356 | | |
| LVLHTVWNGNGMIGF | 266 | 347-361 | | |
| VWNGNGMIGFQVLLS | 267 | 352-366 | 353-371 | External |
| | | | | Loop 6 |
| QVLLSIGELSLAIVI | 268 | 362-376 | | |
| LAIVIALTSGLLTGL | 269 | 372-386 | 372-388 | Transmem- |
| | | | | brane 12 |
| LLTGLLLNLKIWKAP | 270 | 382-396 | | |
| LLNLKIWKAPHVAKY | 271 | 387-401 | 389-417 | Internal |
| | | | | —COOH |
| IWKAPHVAKYFDDQV | 272 | 392-406 | | |
| HVAKYFDDQVFWKFP | 273 | 397-411 | | |
| FDDQVFWKFPHLAVG | 274 | 402-416 | | |
| DDQVFWKFPHLAVGF | 275 | 403-417 | | |

TABLE 5

| | | | |
|---|---|---|---|
| | SEQ | | |
| RhCE PEPTIDE | ID | | |
| SEQUENCE | No. | RESIDUES | FOLDING |

| | | | | |
|---|---|---|---|---|
| SSKYPRSVRRCLPLC | 276 | 2-16 | 2-12 | Internal —NH4 |
| CLPLCALTLEAALIL | 277 | 12-26 | 13-29 | Transmem- |
| | | | | brane 1 |
| AALILLFYFFTHYDA | 278 | 22-36 | | |
| THYDASLEDQKGLVA | 279 | 32-46 | 30-52 | External |
| | | | | loop 1 |
| KGLVASYQVGQDLTV | 280 | 42-56 | | |
| QDLTVMAAIGLGFLT | 281 | 52-66 | 52-70 | Transmem- |
| | | | | brane 2 |
| MAAIGLGFLTSNSRR | 282 | 57-71 | | |
| LGFLTSSFRRHSWSS | 283 | 62-76 | | |
| SSFRRHSWSSVAFNL | 284 | 67-81 | 71-75 | Internal |
| | | | | Loop 1 |
| HSWSSVAFNLFMLAL | 285 | 72-86 | | |
| FMLALGVQWAILLDG | 286 | 82-96 | 76-92 | Transmem- |
| | | | | brane 3 |
| ILLDGFLSQFPSGKV | 287 | 92-106 | | |
| FLSQFPSGKVVITLF | 288 | 97-111 | 93-109 | External |
| | | | | Loop 2 |
| PSGKVVITLFSIRLA | 289 | 102-116 | | |
| VITLFSIRLATMSAM | 290 | 107-121 | | |
| SIRLATMSAMSVLIS | 291 | 112-126 | 110-129 | Transmem- |
| | | | | brane 4 |
| TMSAMSVLISAGAVL | 292 | 117-131 | | |
| SVLISAGAVLGKVNL | 293 | 122-136 | 130-134 | Internal |
| | | | | Loop 2 |
| AGAVLGKVNLAQLVV | 294 | 127-141 | | |
| GKVNLAQLVVMVLVE | 295 | 132-146 | 135-152 | Transmem- |
| | | | | brane 5 |
| MVLVEVTALGTLRMV | 296 | 142-156 | | |
| VTALGTLRMVISNIF | 297 | 147-161 | | |
| TLRMVISNIFNTDYH | 298 | 152-166 | 153-168 | External |
| | | | | Loop 3 |
| ISNIFNTDYHMNLRH | 299 | 157-171 | | |
| NTDYHMNLRHIYVFA | 300 | 162-176 | | |
| MNLRHIYVFAAYFGL | 301 | 167-181 | 169-186 | Transmem- |
| | | | | brane 6 |
| IYVFAAYFGLTVAWC | 302 | 172-186 | | |
| AYFGLTVAWCLPKPL | 303 | 177-191 | | |
| TVAWCLPKPLPKGTE | 304 | 182-196 | 187-211 | Internal |
| | | | | Loop 3 |
| LPKPLKEGTEDKDQR | 305 | 187-201 | | |
| PKGTEDNDQRATIPS | 306 | 192-206 | | |
| DNDQRATIPSLSAML | 307 | 197-211 | | |
| ATIPSLSAMLGALFL | 308 | 202-216 | | |
| LSAMLGALFLWMFWP | 309 | 207-221 | | |
| GALFLWMFWPSVNSP | 310 | 212-226 | 212-229 | Transmem- |
| | | | | brane 7 |
| WMFWPSVNSPLLRSP | 311 | 217-231 | | |
| SVNSPLLRSPIQRKN | 312 | 222-236 | | |
| LLRSPIQRKNAMFNT | 313 | 227-241 | 230-241 | External |
| | | | | Loop 4 |
| IQRKNAMFNTYYALA | 314 | 232-246 | | |
| AMFNTYYALAVSVVT | 315 | 237-251 | | |
| YYAVAVSVVTAISGS | 316 | 242-256 | 242-259 | Transmem- |
| | | | | brane 8 |
| AISGSSLAHPQRKIS | 317 | 252-266 | | |
| SLAHPQRKISMTYVH | 318 | 257-271 | 260-267 | Internal |
| | | | | Loop 4 |
| QRKISMTYVHSAVLA | 319 | 262-276 | | |
| MTYVHSAVLAGGVAV | 320 | 267-281 | 268-285 | Transmem- |
| | | | | brane 9 |
| SAVLAGGVAVGTSCH | 321 | 272-286 | | |
| GTSCHLIPSPWLAMV | 322 | 282-296 | 286-290 | External |
| | | | | Loop 5 |
| WLAMVLGLVAGLISI | 323 | 292-306 | 291-308 | Transmem- |
| | | | | brane 10 |
| LGLVAGLISIGGAKY | 324 | 297-311 | | |
| GLISIGGAKCLPVCC | 325 | 302-316 | | |
| GGAKCLPVCCNRVLG | 326 | 307-321 | 309-335 | Internal |
| | | | | Loop 5 |
| LPVCCNRVLGIHHIS | 327 | 312-326 | | |
| NRVLGIHHISVMHSI | 328 | 317-331 | | |
| IHHISVMHSIFSLLG | 329 | 322-336 | | |
| IMHSIFSLLGLLGEI | 330 | 327-341 | | |
| FSLLGLLGEITYIVL | 331 | 332-346 | 336-352 | Transmem- |
| | | | | brane 11 |
| LLGEITYIVLLVLHT | 332 | 337-351 | | |
| TYIVLLVLHTVWNGN | 333 | 342-356 | | |
| LVLHTVWNGNGMIGF | 334 | 347-361 | | |
| VWNGNGMIGFQVLLS | 335 | 352-366 | 353-371 | External |
| | | | | Loop 6 |
| QVLLSIGELSLAIVI | 336 | 362-376 | | |
| LAIVIALTSGLLTGL | 337 | 372-386 | 372-388 | Transmem- |
| | | | | brane 12 |
| LLTGLLLNLKIWKAP | 338 | 382-396 | | |
| LLNLKIWKAPHVAKY | 339 | 387-401 | 389-417 | Internal |
| | | | | —COOH |
| IWKAPHVAKYFDDQV | 340 | 392-406 | | |
| HVAKYFDDQVFWKFP | 341 | 397-411 | | |
| FDDQVFWKFPHLAVG | 342 | 402-416 | | |
| DDQVFWKFPHLAVGF | 343 | 403-417 | | |

TABLE 6

| | SEQ ID | |
|---|---|---|
| Rh50GP | No. | RESIDUES |
| MRFTFPLMAIVLEIA | 344 | 1-15 |
| VLEIAMIVLFGLFVE | 345 | 11-25 |
| GLFVEYETDQTVLEQ | 346 | 21-35 |
| TVLEQLNITKPTDMG | 347 | 31-45 |
| PTDMGIFFELYPLFQ | 348 | 41-55 |
| YPLFQDVHVMIFVGF | 349 | 51-65 |
| IFVGFGFLMTFLKKY | 350 | 61-75 |
| FLKKYGFSSVGINLL | 351 | 71-85 |
| GINLLVAALGLQWGT | 352 | 81-95 |
| LQWGTIVQGILQSQG | 353 | 91-105 |
| LQSQGQKFNIGIKNM | 354 | 101-115 |
| GIKNMINADFSAATV | 355 | 111-125 |
| SAATVLISFGAVLGK | 356 | 121-135 |
| AVLGKTSPTQMLIMT | 357 | 131-145 |
| MLIMTILEIVFFAHN | 358 | 141-155 |
| FFAHNEYLVSEIFKA | 359 | 151-165 |
| EIFKASDIGASMTIH | 360 | 161-175 |
| SMTIHAFGAYFGLAV | 361 | 171-185 |
| FGLAVAGILYRSGLR | 362 | 181-195 |
| RSGLRKGHENEESAY | 363 | 191-205 |
| EESAYYSDLFAMIGT | 364 | 201-215 |
| AMIGTLFLWMFWPSF | 365 | 211-225 |
| FWPSFNSAIAEPGDK | 366 | 221-235 |
| EPGDKQCRAIVDTYF | 367 | 231-245 |
| VDTYFSLAACVLTAF | 368 | 241-255 |
| VLTAFAFSSLVEHRG | 369 | 251-265 |
| VEHRGKLNMVHIQNA | 370 | 261-275 |
| HIQNATLAGGVAVGT | 371 | 271-285 |
| VAVGTCADMAIHPFG | 372 | 281-295 |
| IHPFGSMIIGSIAGM | 373 | 291-305 |
| SIAGMVSVLGYKFLT | 374 | 301-315 |
| YKFLTPLFTTKLRIH | 375 | 311-325 |
| KLRIHDTCGVHNLHG | 376 | 321-335 |
| HNLHGLPGVVGGLAG | 377 | 331-345 |
| GGLAGIVAVAMGASN | 378 | 341-355 |
| MGASNTSMAMQAAAL | 379 | 351-365 |
| QAAALGSSIGTAVVG | 380 | 361-375 |
| TAVVGGLMTGLILKL | 381 | 371-385 |
| LILKLPLWGQPSDQN | 382 | 381-395 |
| PSDQNCYDDSVYWKV | 383 | 391-405 |

1. A pharmaceutical composition for the prevention of a condition which results from the alloimmunisation or autoimmunity of a subject or the immunosuppression of a response elicited by alloimmunisation or autoimmunity of a subject by tolerisation, said composition comprising an immunologically effective amount of an epitope from a rhesus protein or a peptide fragment, an immunoreactive analogue or derivative or a cross-reaction sequence thereof.

2. A pharmaceutical composition according to claim 1 wherein the rhesus protein selected from the group consisting of RhD, RhcE, Rhce, RhCe or RhCE protein.

3. A pharmaceutical composition according to claim 2 wherein the epitope is selected from the group consisting of at least one SEQ ID numbers 1 to 383.

4. A pharmaceutical composition according to claim 1 wherein the condition is haemolytic disease of the newborn.

5. A pharmaceutical composition according to claim 4 wherein the epitope is selected from the group consisting of at least one of SEQ ID numbers 1, 2, 3, 4, 5, 6, 7, 8, 9, 10, 11, 12, 13, 14, 15, 16, 17, 18, 19, 20, 21, 22, 24, 25, 26, 27, 28, 29, 33, 34, 35, 36, 37, 40, 41, 43, 44, 45, 46, 47, 48, 49, 50, 51, 52, 53, 54, 55, 56, 58, 59, 60, 61, 62, 63, 64, 65, 66, 67, 68, 72, 73, 74, 75, 76, 81, 82, 91, 104, 105, 112, 117, 118, 132, 133, 134, 138, 139, 140, 141, 142, 143, 144, 149, 150, 159, 172, 173, 180, 185, 186, 200, 201, 202, 206, 207, 210, 211, 212, 213, 215, 216, 217, 218, 219, 220, 221, 227, 240, 241, 248, 253, 254, 255, 268, 269, 270, 274, 275, 278, 279, 280, 281, 283, 284, 285, 286, 287, 288, 289, 295, 308, 309, 316, 321, 322, 336, 337, 338, 342, 343.

6. A pharmaceutical composition according to claim 1 wherein the condition is autoimmune haemolytic anaemia.

7. A pharmaceutical composition according to claim 6 wherein the epitope is selected from the group consisting of at least one of SEQ ID numbers 1, 2, 3, 5, 6, 8, 10, 11, 20, 21, 23, 25, 27, 33, 35, 37, 39, 41, 44, 46, 47, 50, 54, 56, 60, 62, 63, 65, 67, 68, 72, 73, 74, 76, 81, 82, 91, 104, 112, 117, 118, 133, 134, 138, 139, 140, 141, 142, 144, 149, 150, 159, 172, 180, 185, 186, 201, 202, 206, 207, 213, 215, 217, 218, 227, 240, 248, 253, 254, 269, 270, 274, 275, 281, 283, 285, 286, 295, 308, 316, 321, 322, 337, 338, 342, 343.

8. A pharmaceutical composition according to claim 1 wherein the epitope is synthesised.

9. A pharmaceutical composition according to claim 3 wherein the epitope is synthesised.

10. A pharmaceutical composition according to claim 5 wherein the epitope is synthesised.

11. A pharmaceutical composition according to claim 7 wherein the epitope is synthesised.

12. A pharmaceutical composition according to claim 1 wherein the epitope is disposed in a pharmaceutically acceptable vehicle.

13. A pharmaceutical composition according to claim 12 wherein said vehicle is in a form selected from the group consisting of an injectable, oral, rectal, topical and spray-uptake form.

14. A pharmaceutical composition according to claim 2 wherein the epitope is disposed in a pharmaceutically acceptable vehicle.

15. A pharmaceutical composition according to claim 14 wherein said vehicle is in a form selected from the group consisting of an injectable, oral, rectal, topical and spray-uptake form.

16. A pharmaceutical composition according to claim 3 wherein the epitope is disposed in a pharmaceutically acceptable vehicle.

17. A pharmaceutical composition according to claim 16 wherein said vehicle is in a form selected from the group consisting of an injectable, oral, rectal, topical and spray-uptake form.

18. A pharmaceutical composition according to claim 4 wherein the epitope is disposed in a pharmaceutically acceptable vehicle.

19. A pharmaceutical composition according to claim 18 wherein said vehicle is in a form selected from the group consisting of an injectable, oral, rectal, topical and spray-uptake form.

20. A pharmaceutical composition according to claim 5 wherein the epitope is disposed in a pharmaceutically acceptable vehicle.

21. A pharmaceutical composition according to claim 20 wherein said vehicle is in a form is selected from the group consisting of an injectable, oral, rectal, topical and spray-uptake form.

22. A pharmaceutical composition according to claim 6 wherein the epitope is disposed in a pharmaceutically acceptable vehicle.

23. A pharmaceutical composition according to claim 22 wherein said vehicle is in a form selected from the group consisting of an injectable, oral, rectal, topical and spray-uptake form.

24. A pharmaceutical composition according to claim 7 wherein the epitope is disposed in a pharmaceutically acceptable vehicle.

25. A pharmaceutical composition according to claim 24 wherein said vehicle is in a form selected from the group consisting of an injectable, oral, rectal, topical and spray-uptake form.

26. A pharmaceutical composition according to claim 8 wherein the epitope is disposed in a pharmaceutically acceptable vehicle.

27. A pharmaceutical composition according to claim 26 wherein said vehicle is in a form selected from the group consisting of an injectable, oral, rectal, topical and spray-uptake form.

28. A pharmaceutical composition according to claim 7 wherein the epitope is disposed in a pharmaceutically acceptable vehicle.

29. A pharmaceutical composition according to claim 28 wherein said vehicle is in a form selected from the group consisting of an injectable, oral, rectal, topical and spray-uptake form.

30. A pharmaceutical composition according to claim 8 wherein the epitope is disposed in a pharmaceutically acceptable vehicle.

31. A pharmaceutical composition according to claim 30 wherein said vehicle is in a form selected from the group consisting of an injectable, oral, rectal, topical and spray-uptake form.

32. A pharmaceutical composition according to claim 9 wherein the epitope is disposed in a pharmaceutically acceptable vehicle.

33. A pharmaceutical composition according to claim 32 wherein said vehicle is in a form selected from the group consisting of an injectable, oral, rectal, topical and spray-uptake form.

34. A method of treating or managing a condition caused by the alloimmunisaton or autoimmunity of a subject by Rh protein, the method comprising administering immunologically effective amount of an epitope from a rhesus protein or a peptide fragment, an immunoreactive analogue or derivative or a cross-reaction sequence thereof to the subject.

35. A method according to claim 34 wherein the Rh protein is selected from the group consisting of RhD, RhcE, Rhce, RhCe or RhCE protein.

36. A method according to claim 34 wherein the epitope is selected from the group consisting of at least one SEQ ID numbers 1 to 383.

37. A method according to claim 34 wherein the condition is autoimmune haemolytic anaemia.

38. A method according to claim 37 wherein the epitope is selected from the group consisting of at least one of SEQ ID numbers 1, 2, 3, 5, 6, 8, 10, 11, 20, 21, 23, 25, 27, 33, 35, 37, 39, 41, 44, 46, 47, 50, 54, 56, 60, 62, 63, 62, 63, 65, 67, 68, 72, 73, 74, 76, 81, 82, 91, 104, 112, 117, 118, 133, 134, 138, 139, 140, 141, 142, 144, 149, 150, 159, 172, 180, 185, 186, 201, 202, 206, 207, 213, 215, 217, 218, 227, 240, 248, 253, 254, 269, 270, 274, 275, 281, 283, 285, 286, 295, 308, 316, 321, 322, 337, 338, 342, 343.

39. A method according to claim 34 wherein the condition is haemolytic disease of the newborn.

40. A method according to claim 38 wherein the epitope is selected from the group consisting of at least one of SEQ ID numbers 1, 2, 3, 4, 5, 6, 7, 8, 9, 10, 11, 12, 13, 14, 15, 16, 17, 18, 19, 20, 21, 22, 24, 25, 26, 27, 28, 29, 33, 34, 35, 36, 37, 40, 41, 43, 44, 45, 46, 47, 48, 49, 50, 51, 52, 53, 54, 55, 56, 58, 59, 60, 61, 62, 63, 64, 65, 66, 67, 68, 72, 73, 74, 75, 76, 81, 82, 91, 104, 105, 112, 117, 118, 132, 133, 134, 138, 139, 140, 141, 142, 143, 144, 149, 150, 159, 172, 173, 180, 185, 186, 200, 201, 202, 206, 207, 210, 211, 212, 213, 215, 216, 217, 218, 219, 220, 221, 227, 240, 241, 248, 253, 254, 255, 268, 269, 270, 274, 275, 278, 279, 280, 281, 283, 284, 285, 286, 287, 288, 289, 295, 308, 309, 316, 321, 322, 336, 337, 338, 342, 343.

41. A method according to claim 34 wherein the epitope is administered through a route selected from the group consisting of transdermally, transmucosally and orally.

42. A method according to claim 35 wherein the epitope is administered through a route selected from the group consisting of transdermally, transmucosally and orally.

43. A method according to claim 36 wherein the epitope is administered through a route selected from the group consisting of transdermally, transmucosally and orally.

44. A method according to claim 37 wherein the epitope is administered through a route selected from the group consisting of transdermally, transmucosally and orally.

45. A method according to claim 38 wherein the epitope is administered through a route selected from the group consisting of transdermally, transmucosally and orally.

46. A method according to claim 39 wherein the epitope is administered through a route selected from the group consisting of transdermally, transmucosally and orally.

47. A method according to claim 40 wherein the epitope is administered through a route selected from the group consisting of transdermally, transmucosally and orally.
